SEQUENCE LISTING (69 sequences)

SEQ ID NO: 1 - protein, 425 aa, Hilicobacter pylori
MFQPLLDAFIESASIEKMASKSPPPPLKIAVANWWGDEEIKEFKKSVLYFILSQRYAITLHQNPNEFSDLVFSNPLGAARKILSYQNTKRVFYTGENESPNFNLFDYAIGFDELDFNDRYLRMPLYYAHLHYKAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL

SEQ ID NO: 2 - protein, 396 aa, Serratia marcescens
MQRLSRLSLRINPIFAAFLLIAFLSGIAGALLTPTLSLFLTTEVKVRPLWVGLFYTANAVAGIVVSFLLAKRSDTRGDRRRLILLCCLMAVGNCLLFAFNRDYLTLITAGVLMSAVANTAMPQIFALAREYADSEAREVVMFSSVMRAQLSLAWVIGPPLSFALALNYGFTVMFLIAAVTFAVCVLLVGFMLPSVPRAAENEGLQGGVSAPIAPASAWRNRDVRLLFIASMLMWTCNTLYIIDMPLYITADLGLPEGLAGVLMGTAAGLEIPAMLLAGYYVKRFGKRNMMLLAVVAGVLFYLGLTVLESKPALIALQLLNAVFIGIVAGIGMLYFQDLMPGRPGAATTLFTNSISTGVILAGVLQGALVENLGHGSVYWMAALLALAALGMSAKVR

SEQ ID NO: 3 - protein, 417 aa, Escherichia coli [sequence omitted]

SEQ ID NO: 4 - protein, 393 aa, Bifidobacterium longum
MTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGG

SEQ ID NO: 5 - protein, 824 aa, Bifidobacterium longum
MAASNPISWSQRTFPSPEGTIACRFRAHADGRIFDAVNGSANDAPLLICAIEHDALRVRATTPRQHVDFDIEDTTGIADGAMHTFALTFGEFGTRVYLDGSQCFSGTANLCPTTLTGTEGSGQGAIRLAGPSIDVTDMRLHAIPLTSESIAALTPRPAPDIDFAAAQLAPRDVRRVRTLRSGTIFMHFRVRGPRQYGTLLAAGERGEERLAVSIDDNGITMTAADGLYEPSTYHARGAWDDGRWHDLSIRSARGAIDMYVDGWHELHQAGQVFFGDWPQLDEVAIGQNTEGVRLMGEVRNGGVFTTPLTDGAIRRLSDAPALTTTALFDKGYHGSVSYRIPSIIRTPHGVVVAGADQRTAIANDAPNHINFVMRRSLDGGRTWLDMQTVIANPGEGVDGACTIDSCLVCDERNGRLTVLIDRFAGGVGLPNNTPGTGVDRHGRPCLYDRAGTRYVLADDGTVLDGGGERTGYRVDAHGNVTHEGRASGNIYLKEGADPDESLLIERTSFIIELHSDDDGETWSTPRNINHMIKEDWMHFLGVSPGNGIQLQASEHRGRLLVPFYCTGASLKHYSGGALISDDGGDTWRRGSMINDGRIVNGTAVDPKNIRDDDATTHESVFVERADGTVVCFFRNQNHAGRIGVALSHDGGETWDDLYFDKDVPDIFCQPNAVACAPRSDTMVFANASQMLPYRGNGVLRLSLDGARTWAAHRCINPYHYGYQCMTMLPDGELGLLWERETAGLYFTTLPLSVFGAAETHSADPEPKPEPKPEPEKPAGPKVDVPTEKPGLSKTGASTAGMSIVFVLLALSGVAALSLRRRSVH

SEQ ID NO: 6 - protein, 836 aa, Bifidobacterium bifidum
MVRSTKPSLLRRLGALVAAAAMLVVLPAGVSTASAASDDADMLTVTMTRTDTLGDEVYVGDTLTYSFTNTNNTSSAFTAFPAESNLSGVLTTGTPNCRYENLAGGASYPCSTASHTITADDLTAGSFTPRTVWKATSDRGGTQVLQDNIVSTGDTVTVKEGKRPDPATIPTDRADGEAVRLATARQNLGTECYRIPALAEAPNGWILAAFDQRPNTAMANGSGVKCWDAPQPNSIVQRISKDGGKSWTPIQYVAQGKNAPERYGYSDPSYVVDEETGEIFLFFVHSYNKGFADSQLGVDESNRNVLHAVVVSSKDNGETWSKPRDITADITKGYENEWKSRFATSGAGIQLKYGKYKGRLIQQYAVGRTTGSNAAVSVYSDDHGKTWQAGNPVTGMLMDENKVVELSDGHVMLNSRPGNGSGYRRVAISEDGGVNYGTVKNETQLPDPNNNAHITRAFPNAPEGSAKAKVLLYSSPRANNEGRANGVVRISLDDGTTWSSGKLYKAGSMAYSVITALSGAAGGGYGLLYEGAWVTGGGIDSHDIMYTHISMDWLGYLSATADDVTASVEEGASTVDVTVPVSNVGSVDYTGVTVTPADLPTGWSASPVNVGALASGASKDVTVTVNVPATAKKDDVAKIVLRVTGTSAANADATTGFDGSITVNVTEKSEPDPEPEPTITGVSAVTSQAGVKVGDVFDASKVSVTAAMSDGSSKALAAGEYSLSAVDADGKAVDLAEPFAAAGVVTVTVSVPVEGADPLTASFTIDVAEKSADPEPKPEPEPKPEPEKPAGPKVDVPTEKPGLSKTGASTAGMSIVFVLLALSGVAALSLRRRSVH

SEQ ID NO: 7 - protein, 1795 aa, Bifidobacterium bifidum
MTTIFRRATAKTLMRKLSGLLVAIAMLAVLPAGTISANAADEPPQEYLQLTLTRTDSNGTPAEVGDKLTYSLGYKNVSDTGFIVHPTASNLNNVATPQSASNPNPMCRWGNLAAGASAACTWSASKEFAYHVVTEDDVANGFTPTATVSATTQDGTNGVLQSVDITGETVPAVPATSTLRVAMQRTDTLGDNVKIGDRLTFNFTYTNKTAQKIYAYPSESNIERVDVVSFPRNSCRSGVEANQTASCGFAYHVITAEDVVARRYTPTATFRATSDRDGTQVLQDDMTFTTGTVTVAGPADDAASTPTERKDGEPLLLATNKQIGNTDYYRIPAIAQAPNGWILAAWDLRPKLAADAPNPNSIVQRISKDGGKSWETLAYVAQGRSATNKYGYSDPSYVVDEEAGKIFLFCVKSYDQGYFGSVLGVEDARNVLQAVVMESDDNGATWSEPRNITKDITKGHEDEWKSRFASSGHGIQLKYGQYKGRLIQQYAVRTTSNTNIAVSVYSDDHGKTWKAGNPVTEVNMDENKVVELSDGRVMLNSRPGAAGYRRVAISEDGGVNYGPIKSETQLPDPNNNAQITRAFPNAPEGSAKAKVLLYSAPRASNEGRANGVVRVSFDDGTTWSAGKLFKAGSMAYSVITALNDAAGGGYGLLYEGESITYTRASMEWLGYLTATASGTAAVKEGEGTLTAPVTVTNDGLTDYTNVTVTPTGLPSGWSAEAVNVGNLAAGQSATVNVPVTVPAAAVSGTVAKATMKITGKYAQSEDTLHSFAEGELAVTVTDPDPAAKRLKLTIERTDDNGDPVKVGDTLTYRITYENVGTQSFAVYPRESNLDGVTTPQSASNPAPVCRWSRLAPGATGACVSGNGKQLAYHTVTEADATAGSFTPSATIDATADASGETVLESVSITGDPVTVSQPVELPADIAAWKTRNEALADWQTLSEKLAKTDRINWLFTGDSITHGVQFTRGYRTYSELFANHLDTASVRGVSRANDVVMNTGISSADASWPLKDGAFEKWVSDKHPDVVFLTFGMNDGRTGQAFTVDQYTANLSTLIDKIRDLGAIPVLQTQNYTTNTTFNANLDTYFDAERRLALDKNVLLVDFNKQWLELGGGNRESGTYMGAGNDIHPGENGHIEWAKFTLGALNMIANDDPLARWSSSDTTLDKPTVTVDADGNGLKGSDGLEPAPAAAKSVGKFLSGAQYVDLGGDVVSAVAGKRESNVTIRFRASATGQPQTLFSLGDSDSATRATVRLSATGLVQFLNSGNTGDFYTVGTNDLADGAWHTVSVNFVANGFTIYVDGAAMRAISGGAGTQLNVPGAITVNTATAGAIRGADSAGGAQQLTGIVDYVAAWSRTLTDAEAKRISAETSAVAVTKVDAAVNALQPIISDTGARKNIVFVGGETIEGGYTDHLIAKNIVQLLDERVRWEYVTGLSATDRERQRAKFFVAAGQGGLTAKQMDEDYAAMVGEYSPDILFLAPDLYDADGNLAESAAAAFAGHIRSVAAKAKEAGAKVVLVTPVTVRGGEDEYAGAMRTVAKEDDLPLIDAQAWIGKVVAADASVKTAWFNKAGQLNYAGHLGYARFMMRSLDLYPSNVSGSRIASLPYDTANVTLVGASENGGELPVGRVEGTDRAHIDTMQIGAAASLVVVDSYAVYEIGEDGGRTLVADGLKPADVLADGIDVTVNDTAAHRYEVVGSANVPEGADAVTVTYTATLAAVEEPEPGPDPDPTPDPSEKPDGDGTGDGTGAGTGDVQKPTPDAVAKTGADVFGLLTAVAALLAAGGVTLSLRRRANR

SEQ ID NO: 8 - protein, 1795 aa, Bifidobacterium bifidum
MTTIFRRATAKTLMRKLSGLLVAIAMLAVLPAGTISANAADEPPQEYLQLTLTRTDSNGTPAEVGDKLTYSLGYKNVSDTGFIVHPTASNLNNVATPQSASNPNPMCRWGNLAAGASAACTWSASKEFAYHVVTEDDVANGFTPTATVSATTQDGTNGVLQSVDITGETVPAVPATSTLRVAMQRTDTLGDNVKIGDRLTFNFTYTNKTAQKIYAYPSESNIERVDVVSFPRNSCRSGVEANQTASCGFAYHVITAEDVVARRYTPTATFRATSDRDGTQVLQDDMTFTTGTVTVAGPADDAASTPTERKDGEPLLLATNKQIGNTDYYRIPAIAQAPNGWILAAWDLRPKLAADAPNPNSIVQRISKDGGKSWETLAYVAQGRSATNKYGYSDPSYVVDEEAGKIFLFCVKSYDQGYFGSVLGVEDARNVLQAVVMESDDNGATWSEPRNITKDITKGHEDEWKSRFASSGHGIQLKYGQYKGRLIQQYAVRTTSNTNIAVSVYSDDHGKTWKAGNPVTEANMDENKVVELSDGRVMLNSRPGAAGYRRVAISEDGGVNYGPIKSETQLPDPNNNAQITRAFPNAPEGSAKAKVLLYSAPRASNEGRANGVVRVSFDDGTTWSAGKLFKEGSMAYSVITALNDAAGGGYGLLYEGESITYTRVSMEWLGYLTATASGTATVKEGEGTLTAPVTVTNDGLTDYTNVTVTPTGLPSGWSAEAVNVGNLAAGQSATVNVPVTVPAAAVSGTVAKATMKITGKYAQSEDTLHSFAEGELAVTVTEPDPAAKRLKLTIERTDDNGAPVKVGDTLTYRITYENVGTQSFAVYPRKSNLDGVTTPQSASNPAPVCRWSRLDPGTTGACVSGNGKRLAYHTVTEADATAGSFTPSATIDATADTSGETVLESVSITGDPVTVSQPVELPADIAAWKTRNEALDDWQTLSEKLAKTDRINWLFTGDSITHGVQLTRGYRTYSELFANHLDTASVRGVSRANDVVMNTGISSADASWPLKDGAFEKWVSDKHPDVVFLTFGMNDGRTGQAFTVDQYTANLSTLIDKIRDLGAIPVLQTQNYTTNTTFNANLDTYFDAERRLALDKNVLLVDFNKQWLELGGGNRESGTYMGAGNDIHPGENGHIEWAKFTLGALNMIANDDPLARWSSSDTTLDKPTVTVDADGNGLKGSDGLEPAPAAAKSVGKFLSGAQYVDLGGDVVSAVAGKRESNVTIRFRASATGQPQTLFSLGDSDSATRATVRLSATGLVQFLNSGNTGDFYTVGTNDLADGAWHTVSVNFVANGFTIYVDGAAMRAISGGAGTQLNVPGAITVNTATAGAIRGADSAGGAQQLTGIVDYVAAWSRTLTDAEAKRISAETSAVAVTKVDAAVNALQPIISDTGARKNIVFVGGETIEGGYTDHLIAKNIVQLLDERVRWEYVTGLSATDRELQRAKFFVAAGQGGLTAKQMDEDYAAMVGEYSPDILFLAPDLYDADGILAESDAAAFAGHIRSVAAKAKEAGAKVVLVTPVTVRGGEDEYAGAMRTVAKEDDLPLIDAQAWIGKVVAADASVKTAWFNKAGQLNYAGHLGYARFMMRSLDLYPSNVSGSRIASLPYDTANVTLVGASENGGELPVGRVEGTDRAHIDTMQIGAAASLVVTDSYAVYEIGEDGGRTLVADGLKPADVLADGIDVTVNDTAAHRYEVVGSANVPEGADAVTVTYTATLAAVEEPEPGPDPDPTPDPSEKPDGDGTGDGTGAGAGDVQKPTPDAVAKTGADVFGLLAAVAVLLAAGGVTLSLRRRANR

SEQ ID NO: 9 - protein, 834 aa, Bifidobacterium bifidum
MVRSTKPSLLRRFGALVAAAAMLVVLPAGVSTASAASDDADMLTVTMTRTDALGDEVYVGDTLTYSFTNTNNTSSAFTAFPAESNLSGVLTTGTPNCRYENLAGGASYPCSTASHTITADDLTAGSFTPRTVWKATSDRGGTQVLQDNIVSTGDTVTVKEGKRPDPATIPTDRADGEAVRLATARQNLGTECYRIPALAEAPNGWILAAFDQRPNTAMANGSGVKCWDAPQPNSIVQRISKDGGKSWTPIQYVAQGKNAPERYGYSDPSYVVDKETGEIFLFFVHSYNKGFADSQLGVDESNRNVLHAVVVSSKDNGETWSKPRDITADITKGYENEWKSRFATSGAGIQLKYGKYKGRLIQQYAVGRTTGSNAAVSVYSDDHGKTWQAGNPVTGMLMDENKVVELSDGRVMLNSRPGNGSGYRRVAISEDGGVNYGTVKNETQLPDPNNNAHITRAFPNAPEGSAKAKVLLYSSPRANNEGRANGVVRISLDDGTTWSSGKLYKEGSMAYSVITALSGAAGGGYGLLYEGAWVTGGGIDSHDIMYTHISMDWLGYLSATADDVTASVEEGASTVDVTVPVSNVGSVDYTGVTVTPADLPTGWSASPVNVGALASGASKDVTVTVNVPATAKKDDVAKIVLRVTGTSAANANATTGFDGSITVNVTEKSEPDPEPEPTITGVSAVTSQAGVKVGDVFDASKVSVTAAMSDGSSKALAAGEYSLSAVDADGKAVDLAEPFAAAGVVTVTVSVPVEGADPLTASFTIDVAEKSADPEPKPEPKPEPEKPAGPKVDVPTEKPGLSKTGASTAGMSIVFVLLALSGVAALSLRRRSVH

SEQ ID NO: 10 - protein, 834 aa, Bifidobacterium bifidum
MVRSTKPSLLRRLGALVAAAAMLVVLPAGVSTASAASDDADMLTVTMTRTDALGDEVYVGDTLTYSFTNTNNTSSAFTAFPAESNLSGVLTTGTPNCRYENLAGGASYPCSTASHTITADDLTAGSFTPRTVWKATSDRGGTQVLQDNIVSTGDTVTVKEGKRPDPATIPTDRADGEAVRLATARQNLGTECYRIPALAEAPNGWILAAFDQRPNTAMANGSGVKCWDAPQPNSIVQRISKDGGKSWTPIQYVAQGKNAPERYGYSDPSYVVDEETGEIFLFFVHSYNKGFADSQLGVDESNRNVLHAVVVSSKDNGETWSKPRDITADITKGYENEWKSRFATSGAGIQLKYGKYKGRLIQQYAVGRTTGSNAAVSVYSDDHGKTWQAGNPVTGMLMDENKVVELSDGRVMLNSRPGNGSGYRRVAISKDGGVNYGTVKNETQLPDPNNNAHITRAFPNAPEGSAKAKVLLYSSPRANNEGRANGVVRISLDDGTTWSSGKLYKAGSMAYSVITALSGAAGGGYGLLYEGAWVTGGGIDSHDIMYTHISMDWLGYLSATADDVTASVEEGASTVDVTVPVSNIGSVDYTGVTVTPADLPTGWSASPVNVGALASGASKDVTVTVNVPATAKKDDVAKIVLRVTGTSAANADATTGFDGSITVNVTEKSEPDPEPEPTITGVSAVTSQAGVKVGDVFDASKVSVTAAMSDGSSKALAAGEYSLSAVDADGKAVDLAEPFAAAGVVTVTVSVPVEGANPLTASFTIDVAEKSVDPEPKPEPKPEPEKPAGPKVDVPTEQPGLSKTGASTAGMSIVFVLLALSGVAALSLRRRSAH

SEQ ID NO: 11 - protein, 1782 aa, Bifidobacterium bifidum
MRKLSGLLVAIAMLAVLPAGTISANAADEPPQEYLQLTLTRTDSNGTPAEVGDKLTYSLGYKNVSDTGFIVHPTASNLNNVATPQSASNPNPMCRWGNLAAGASAACTWSASKEFAYHVVTEDDVANGFTPTATVSATTQDGTNGVLQSVDITGETVPAVPATSTLRVAMQRTDTLGDNVKIGDRLTFNFTYTNKTAQKIYAYPSESNIERVDVVSYPRNSCRSGVEANQTASCGFAYHVITAEDVVARRYTPTATFRATSDRDGTQVLQDDMTFTTGTVTVAGPADDAASTPTERKDGEPLLLATNKQIGNTDYYRIPAIAQAPNGWILAAWDLRPSSAADAPNPNSIVQRISKDGGKSWETLAYVAQGRSATNKYGYSDPSYVVDEEAGKIFLFCVKSYDQGYFGSVLGVEDARNVLQAVVMESDDNGATWSEPRNITKDITKGHEDEWKSRFASSGHGIQLKYGQYKGRLIQQYAVRTTSNTNIAVSVYSDDHGKTWKAGNPVTEVNMDENKVVELSDGRVMLNSRPGAAGYRRVAISEDGGVNYGPIKSETQLPDPNNNAQITRAFPNAPEGSAKAKVLLYSAPRASNEGRANGVVRVSFDDGTTWSAGKLFKEGSMAYSVITALNDAAGGGYGLLYEGESITYTRASMEWLGYLTATASGTATVKEGEGTLTAPVTVTNDGLTDYTNVNVTPTGLPSGWSAEAVNVGNLAAGQSATVNVPVTVPAAAVSGTVAKATMKITGKYAQSEDTLHSFAEGELAVTVTDPDPAAKRLKLTIERTDDNGDPVKVGDTLTYRITYENVGTQSFAVYPRESNLDGVTTPQSASNPAPVCRWSRLAPGTTGACVSGNGKQLAYHTVTEADATAGSFTPSATIDATADASGEMVLESVSITGDPVTVSQPVELPADIAAWKTRNEALDDWQALSEKLAKTDRINWLFTGDSITHGVQFTRGYRTYSELFANHLDTASVRGVSRANDVVMNTGISSADASWPLKDGAFEKWVSDKHPDVVFLTFGMNDGRTGQAFTVDQYTANLSTLIDKIRDIGAIPVLQTQNYTTNTTFNANLDTYFDAERRLALDKNVLLVDFNKQWLALGGGNRESGTYMGAGNDIHPGENGHIEWAKFTLGALNMIANDDPLARWSSSDTTLDKPTVTVDADGNGLKGSDGLEPAPAAAKSVGKFLSGAQYVDLGGDVVSAVAGKRESNVTIRFRASATGQPQTLFSLGDSDSATRATVRLSATGLVQFLNSGNTGDFYTVGTNDLADGAWHTVSVNFVANGFTIYVDGAAMRAISGGTGTQLNVPGAITVNTATAGAIRGADSAGGAQQLTGIVDYVAAWSRTLTGAEAKRISAETSAVAVTKVDAAVNALQPIISDTGARKNIVFVGGETIEGGYTDHLIAKNIVQLLDERVRWEYVTGLSATDRERQRAKFFVAAGQGGLTAKQMDEDYAAMVGEYSPDILFLAPDLYDADGNLAESDAAAFAGHIRSVAAKAKEAGAKVVLVTPVTVRGGEDEYAGAMRTVAKEDDLPLIDAQAWIGKVVAADASVKTAWFNKAGQLNYAGHLGYARFMMRSLDLYPSNVSGSRIASLPYDTANVTLVGASENGGELPVGRVEGTDRAHIDTMQIGAAASLVVADSYAVYEIGEDGGRTLVADGLKPADVLADGIDVTVNDTAAHRYEVVGSANVPEGADAVTVTYTATLAAVEEPEPGPDPDPTPDPSEKPDGDGTGDGTGAGTGDVQKPTPDAVAKTGADVFGLLAAVAVLLAAGSVTLSLRRRANR

SEQ ID NO: 12 - protein, 835 aa, Bifidobacterium bifidum
MVRSTKPSLLRRFGALVAAAAMLVVLPAGVSTASAASDDADMLTVTMTRTDALGDEVYVGDTLTYSFTNTNNTSSAFTAFPAESNLSGVLTTGTPNCRYENLAGGASYPCSTASHTITADDLTAGSFTPRTVWKATSDRGGTQVLQDNIVSTGDTVTVKEGKRPDPATIPTDRADGEAVRLATARQNLGTECYRIPALAEAPNGWILAAFDQRPNTAMANGSGVKCWDAPQPNSIVQRISKDGGKSWTPIQYVAQGKNAPERYGYSDPSYVVDKETGEIFLFFVHSYNKGFADSQLGVDESNRNVLHAVVVSSKDNGETWSKPRDITADITKGYENEWKSRFATSGAGIQLKYGKYKGRLIQQYAVGRTTGSNAAVSVYSDDHGKTWQAGNPVTGMLMDENKVVELSDGRVMLNSRPGAAGYRRVAISEDGGVNYGTVKNETQLPDPNNNAHITRAFPNAPEGSAKAKVLLYSSPRANNEGRANGVVRISLDDGTTWSSGKLYKEGSMAYSVITALSDAAGGGYGLLYEGAWVTGGGIDSHDIMYTHISMDWLGYLSATADDVTASVEEGASTVDVTVPVSNVGSVDYTGVTVTPADLPTGWSASPVNVGALASGTSKDVTVTVNVPATAKKDDVAKIVLRVTGTSAANANATTGFDGSITVNVTEKSEPDPEPEPTITGVSAVTSQAGVKVGDVFDASKVSVTAAMSDGSSKALAAGEYSLSAVDADGKAVDLAEPFAAAGVVTVTVSVPVEGAGPLTASFTIDVAEKSVDPEPKPEPEPKPEPEKPAGPKVDVPTEQPGLSKTGASTAGMSIVFVLLALSGIAALSLRRRSVH

SEQ ID NO: 13 - protein, 1060 aa, Trypanosoma cruzi
MGKTVVGASRMFWLMFFVPLLLALCPSEPAHALAPGSSRVELFKRQSSKVPFEKGGKVTERVVHSFRLPALVNVDGVMVAIADARYETSNDNSLIDTVAKYSVDDGETWETQIAIKNSRASSVSRVVDPTVIVKGNKLYVLVGSYNSSRSYWTSHGDARDWDILLAVGEVTKSTAGGKITASIKWGSPVSLKEFFPAEMEGMHTNQFLGGAGVAIVASNGNLVYPVQVTNKKKQVFSKIFYSEDEGKTWKFGEGRSDFGCSEPVALEWEGKLIINTRVDYRRRLVYESSDMGNSWVEAVGTLSRVWGPSPKSNQPGSQSSFTAVTIEGMRVMLFTHPLNFKGRWLRDRLNLWLTDNQRIYNVGQVSIGDENSAYSSVLYKDDKLYCLHEINSNEVYSLVFARLVGELRIIKSVLQSWKNWDSHLSSICTPADPAASSSERGCGPAVTTVGLVGFLSHSATKTEWEDAYRCVNASTANAERVPNGLKFAGVGGGALWPVSQQGQNQRYHFANHAFTLVASVTIHEVPSVASPLLGASLDSSGGKKLLGLSYDEKHQWQPIYGSTPVTPTGSWEMGKRYHVVLTMANKIGSVYIDGEPLEGSGQTVVPDGRTPDISHFYVGGYGRSDMPTISHVTVNNVLLYNRQLNAEEIRTLFLSQDLIGTEAHMGSSSGSSAHSTPSTPADNGAHSTPSTPADSSAHSTPSTPADSSAHSTPSAPGDNGAHSTPSTPGDSSAHSTPSTPADNGAHSTPSAPADSNAHSTPSTPADNGAHSTPSTPADNGAHSTPSTPGDNGAHSTPSTPGDSSAHSTPSTPADNGAHSTPSAPADSNAHSTPSTPGDNGAHSTPSAPADSNAHSTPSTPADSSAHSTPSAPGDNGAHSTPSAPADSSAHSTPSAPGDNGAHSTPSAPADNGAHSTPSAPGDSNAHSTPSTPADSSAHSTPSTPADSSAHSTPSAPGDNGAHSTPSAPADSSAHSTPSIPGDSSAHSTPSAPADSSAHSTPSAPGDNGAHSTPSTPADNGANGTVLILHDGAAFSAFSGGGLLLCAGALLLHVFVMAVFF

SEQ ID NO: 14 - protein, 642 aa, Trypanosoma cruzi
MLAPGSSRVELFKRQSSKVPFEKDGKVTERVVHSFRLPALVNVDGVMVAIADARYETSNDNSLIDTVAKYSVDDGETWETQIAIKNSRASSVSRVVDPTVIVKGNKLYVLVGSYNSSRSYWTSHGDARDWDILLAVGEVTKSTAGGKITASIKWGSPVSLKEFFPAEMEGMHTNQFLGGAGVAIVASNGNLVYPVQVTNKKKQVFSKIFYSEDEGKTWKFGKGRSAFGCSEPVALEWEGKLIINTRVDYRRRLVYESSDMGNSWLEAVGTLSRVWGPSPKSNQPGSQSSFTAVTIEGMRVMLFTHPLNFKGRWLRDRLNLWLTDNQRIYNVGQVSIGDENSAYSSVLYKDDKLYCLHEINSNEVYSLVFARLVGELRIIKSVLQSWKNWDSHLSSICTPADPAASSSERGCGPAVTTVGLVGFLSHSATKTEWEDAYRCVNASTANAERVPNGLKFAGVGGGALWPVSQQGQNQRYRFANHAFTVVASVTIHEVPSVASPLLGASLDSSGGKKLLGLSYDERHQWQPIYGSTPVTPTGSWEMGKRYHVVLTMANKIGSEYIDGEPLEGSGQTVVPDERTPDISHFYVGGYKRSDMPTISHVTVNNVLLYNRQLNAEEIRTLFLSQDLIGTEAHMDSSSDTSA

SEQ ID NO: 15 - protein, 448 aa, Thermotoga maritima
MISMKPRYKPDWESLREHTVPKWFDKAKFGIFIHWGIYSVPGWATPTGELGKVPMDAWFFQNPYAEWYENSLRIKESPTWEYHVKTYGENFEYEKFADLFTAEKWDPQEWADLFKKAGAKYVIPTTKHHDGFCLWGTKYTDFNSVKRGPKRDLVGDLAKAVREAGLRFGVYYSGGLDWRFTTEPIRYPEDLSYIRPNTYEYADYAYKQVMELVDLYLPDVLWNDMGWPEKGKEDLKYLFAYYYNKHPEGSVNDRWGVPHWDFKTAEYHVNYPGDLPGYKWEFTRGIGLSFGYNRNEGPEHMLSVEQLVYTLVDVVSKGGNLLLNVGPKGDGTIPDLQKERLLGLGEWLRKYGDAIYGTSVWERCCAKTEDGTEIRFTRKCNRIFVIFLGIPTGEKIVIEDLNLSAGTVRHFLTGERLSFKNVGKNLEITVPKKLLETDSITLVLEAVE

SEQ ID NO: 16 - protein, 403 aa, Sulfolobus solfataricus
MFTGENWDPYEWAKVFKKSGAKFVVLVAEHHDGFALWESNYTRWCATKIGPKRDIVRELKEAVEGQGLIFGISYHRAEHWWFFDQGMKIESDVKDPRYLDLYGPAQSASLNPRDPPSLDNVQPNDEFLMDWLLRIVEAVEKYRPWLVYFDWWIANPSFQPYLKAFASYYYNRSYKWGIEPVIIYKQGAFGEGTAIPDLAERGTIKNVYPSTWLADTSIDYKSWGYIKDAEYKLPSVILSHLGDVVSKNGVFLLNIGPKADGTIPEEAKRILLDVGDWLNVNGEAIFGSKPWRVYGEGPSGINEGGFFTERKITLGYQDVRYTVKDYYPRQRHIYAILFGKPKEITLRSFMKNLKLIEEAVIVDVSRLDGKGKLEWSLSDEGLKIKIEEVIRAPLVIRVILDYR

SEQ ID NO: 17 - protein, 1959 aa, Bifidobacterium bifidum
MKHRAMSSRLMPLVASCATVGMLLAGLPVSAVAVGTTRAAASDASSSTTATITPSADTTLQTWTSEKNSSMASKPYIGTLQGPSQGVFGEKFESTDAADTTDLKTGLLTFDLSAYDHAPDSATFEMTYLGYRGNPTATDTDTIKVTPVDTTVCTNNATDCGANVATGATKPKFSINDSSFVAESKPFEYGTTVYTGDAITVVPANTKKVTVDVTEIVRQQFAEGKKVITLAVGETKKTEVRFASSEGTTSLNGATADMAPKLTVSVSTKDDLKPSADTTLQAWASEKNEKKNTAAYVGALQPEGDYGDFGEKFKSTDVHDVTDAKMGLMTFDLSDYTAAPEHSILTLTYLGYAGADKTATATDKVKVVAVDTSRCTGTAPCDTNNATWANRPDFEVTDTTKTATSHAFAYGSKKYSDGMTVESGNAKKVLLDVSDVIKAEFAKFSAGATEKKITLALGELNKSDMRFGSKEVTSLTGATEAMQPTLSVTKKPKAYTLSIEGPTKVKYQKGEAFDKAGLVVKATSTADGTVKTLTEGNGEDNYTIDTSAFDSASIGVYPVTVKYNKDPEIAASFNAYVIASVEDGGDGDTSKDDWLWYKQPASQTDATATAGGNYGNPDNNRWQQTTLPFGNGKIGGTVWGEVSRERVTFNEETLWTGGPGSSTSYNGGNNETKGQNGATLRALNKQLANGAETVNPGNLTGGENAAEQGNYLNWGDIYLDYGFNDTTVTEYRRDLNLSKGKADVTFKHDGVTYTREYFASNPDNVMVARLTASKAGKLNFNVSMPTNTNYSKTGETTTVKGDTLTVKGALGNNGLLYNSQIKVVLDNGEGTLSEGSDGASLKVSDAKAVTLYIAAATDYKQKYPSYRTGETAAEVNTRVAKVVQDAANKGYTAVKKAHIDDHSAIYDRVKIDLGQSGHSSDGAVATDALLKAYQRGSATTAQKRELETLVYKYGRYLTIGSSRENSQLPSNLQGIWSVTAGDNAHGNTPWGSDFHMNVNLQMNYWPTYSANMGELAEPLIEYVEGLVKPGRVTAKVYAGAETTNPETTPIGEGEGYMAHTENTAYGWTAPGQSFSWGWSPAAVPWILQNVYEAYEYSGDPALLDRVYALLKEESHFYVNYMLHKAGSSSGDRLTTGVAYSPEQGPLGTDGNTYESSLVWQMLNDAIEAAKAKGDPDGLVGNTTDCSADNWAKNDSGNFTDANANRSWSCAKSLLKPIEVGDSGQIKEWYFEGALGKKKDGSTISGYQADNQHRHMSHLLGLFPGDLITIDNSEYMDAAKTSLRYRCFKGNVLQSNTGWAIGQRINSWARTGDGNTTYQLVELQLKNAMYANLFDYHAPFQIDGNFGNTSGVDEMLLQSNSTFTDTAGKKYVNYTNILPALPDAWAGGSVSGLVARGNFTVGTTWKNGKATEVRLTSNKGKQAAVKITAGGAQNYEVKNGDTAVNAKVVTNADGASLLVFDTTAGTTYTITKKASANVPVTGVTVTGANTATAGDTVTLTATVAPANATDKSVTWSTSDAAVATVNANGVVTTKKAGKVTITATSNGDKTKFGSIEITVSAATVPVTSVTVAGDAAMTVDGEQTLTATVAPATATDKTVTWKSSDATVATVDANGKVVAKKAGEVTITATAGGVSGTLKITVSDKAPTVIPVQSVTVTGKQELVEGASTTLTATVAPADATDKTVTWKSSDESVATVDKDGVVTAKKAGTVTITATAGGVSGTLHITVTAKPVETVPVTSVEVTVEAGTTVSVGKTLQATATVKPGNATNKKVTWKSSDESIATVDANGVITAKKAGKVVITATSTDGTDKSGSVEITVVDETKPTPDHKSVKADTGDVTAGKTGTVTEPKDVAGWKSRSIIKQGKLGKAEIADGTLVYAAGDKTGDDSFVVQYTMADGTVIDVTYSVTVKAAETGKNDGDGKGDGVAKTGAAVGALAGLGLMLLAVGVSVVMIRRKHSA

SEQ ID NO: 18 - protein, 1493 aa, Bifidobacterium bifidum
MLHTASRGCSRSWLRRLTALIAVSALAFVALPNVAVAADPMEYLDVSFGGTFAADTYTTGGDEVAKGPVTKHGSIPTKLDGGGITLAGGTNGVTFTSTASFSESGKVNKGFRAEMEYRTTQTPSNLATLFSAMGNIFVRANGSNLEYGFSTNPSGSTWNDYTKSVTLPSNNVKHIIQLTYLPGADGAASTLQLSVDGVAGETATSAAGELAAVSDSVGNKFGIGYEVNPASGAASRGLAGDVFRARVADSDAPWEILDASQLLHVNFNGTFSGTSYTAASGEQMLGSLVSRSANPSISNSAVTLGGGTAGFDFTPTDFTLGDNEAITRPLVAELRFTPTQTGDNQTLFGAGGNLFLRYESNKLVFGASTKSGDNWTDHKIESAAATGAEHVVSVAYVPNKAGTGAKLVMRVDGGDAQTKDITGLAYLNSSIKGKVGFGNDVHTDALSRGFVGSLSEIRLAETSANFTTNEFKLVYSQVSCDTSGIKEANTFDVEPAECEAALKTKLSKLRPTEGQADYIDWGQIGFLHYGINTYYNQEWGHGNEDPSRINPTGLDTDQWAKSFADGGFKMIMVTVKHHDGFELYDSRYNTEHDWANTAVAKRTGEKDLFRKIVASAKKYGLKVGIYYSPADSYMERKGVWGNNSARVERTIPTLVENDDRAGKVASGKLPTFKYKATDYGAYMLNQLYELLTEYGDISEVWFDGAQGNTAGTEHYDYGVFYEMIRRLQPQAIQANAAYDARWVGNEDGWARQTEWSPQAAYNDGVDKVSLKPGQMAPDGKLGSMSSVLSEIRSGAANQLHWYPAEVDAKNRPGWFYRASQSPASVAEVVKYYEQSTGRNSQYLLNVPPSDTGKLADADAAGLKGLGEELARRYGTDLALGKSATVAASANDTAVAAPKLTDGSKLSSDKAVGNTPTYTIDLGSTVAVDAVKISEDVRNAGQQIESATLQGRVNGTWTNLATMTTVGQQRDLRFTSQNIDAIRLVVNSSRGPVRLSRLEVFHTESEIQTGARAYYIDPTAQTAGDGFTKDKPMTSIEQLHDVTVAPGSVIFVKAGTELTGDFAVFGYGTKDEPITVTTYGESDKATTASFDGMTAGLTLKQALKALGKDDAGWVVADSATAPASRVYVPQDEISVHAQSSQNSGAEAARALDGDSSTSWHSQYSPTTASAPHWVTLDLGKSRENVAYFDYLARIDGNNNGAAKDYEVYVSDDPNDFGAPVASGTLKNVAYTQRIKLTPKNGRYVKFVIKTDYSGSNFGSAAEMNVELLPTAVEEDKVATPQKPTVDDDADTYTIPDIEGVVYKVDGKVLAAGSVVNVGDEDVTVTVTAEPADGYRFPDGVTSPVTYELTFTKKGGEKPPTEVNKDKLHATITKAQAIDRSAYTDESLKVLDDKLAAALKVYDDDKVSQDDVDAAEAALSAAIDALKTKPTTPGGEGEKPGEGEKPGDGNKPGDGKKPGDVIAKTGASTMGVVFAALAMVAGAVVTLEAKRKSNR

SEQ ID NO: 19 - protein, 1487 aa, synthetic construct
  REGION 1..1487: note = Substitution variant of position 759-772 in gi|242345155 (SEQ ID NO: 18)
MLHTASRGCSRSWLRRLTALIAVSALAFVALPNVAVAADPMEYLDVSFGGTFAADTYTTGGDEVAKGPVTKHGSIPTKLDGGGITLAGGTNGVTFTSTASFSESGKVNKGFRAEMEYRTTQTPSNLATLFSAMGNIFVRANGSNLEYGFSTNPSGSTWNDYTKSVTLPSNNVKHIIQLTYLPGADGAASTLQLSVDGVAGETATSAAGELAAVSDSVGNKFGIGYEVNPASGAASRGLAGDVFRARVADSDAPWEILDASQLLHVNFNGTFSGTSYTAASGEQMLGSLVSRSANPSISNSAVTLGGGTAGFDFTPTDFTLGDNEAITRPLVAELRFTPTQTGDNQTLFGAGGNLFLRYESNKLVFGASTKSGDNWTDHKIESAAATGAEHVVSVAYVPNKAGTGAKLVMRVDGGDAQTKDITGLAYLNSSIKGKVGFGNDVHTDALSRGFVGSLSEIRLAETSANFTTNEFKLVYSQVSCDTSGIKEANTFDVEPAECEAALKTKLSKLRPTEGQADYIDWGQIGFLHYGINTYYNQEWGHGNEDPSRINPTGLDTDQWAKSFADGGFKMIMVTVKHHDGFELYDSRYNTEHDWANTAVAKRTGEKDLFRKIVASAKKYGLKVGIYYSPADSYMERKGVWGNNSARVERTIPTLVENDDRAGKVASGKLPTFKYKATDYGAYMLNQLYELLTEYGDISEVWFDGAQGNTAGTEHYDYGVFYEMIRRLQPQAIQANAAYDARWVGNEDGWARQTEWSPQDIEKMKERENPTYLNNGGSMSSVLSEIRSGAANQLHWYPAEVDAKNRPGWFYRASQSPASVAEVVKYYEQSTGRNSQYLLNVPPSDTGKLADADAAGLKGLGEELARRYGTDLALGKSATVAASANDTAVAAPKLTDGSKLSSDKAVGNTPTYTIDLGSTVAVDAVKISEDVRNAGQQIESATLQGRVNGTWTNLATMTTVGQQRDLRFTSQNIDAIRLVVNSSRGPVRLSRLEVFHTESEIQTGARAYYIDPTAQTAGDGFTKDKPMTSIEQLHDVTVAPGSVIFVKAGTELTGDFAVFGYGTKDEPITVTTYGESDKATTASFDGMTAGLTLKQALKALGKDDAGWVVADSATAPASRVYVPQDEISVHAQSSQNSGAEAARALDGDSSTSWHSQYSPTTASAPHWVTLDLGKSRENVAYFDYLARIDGNNNGAAKDYEVYVSDDPNDFGAPVASGTLKNVAYTQRIKLTPKNGRYVKFVIKTDYSGSNFGSAAEMNVELLPTAVEEDKVATPQKPTVDDDADTYTIPDIEGVVYKVDGKVLAAGSVVNVGDEDVTVTVTAEPADGYRFPDGVTSPVTYELTFTKKGGEKPPTEVNKDKLHATITKAQAIDRSAYTDESLKVLDDKLAAALKVYDDDKVSQDDVDAAEAALSAAIDALKTKPTTPGGEGEKPGEGEKPGDGNKPGDGKKPGDVIAKTGASTMGVVFAALAMVAGAVVTLEAKRKSNR

SEQ ID NO: 20 - protein, 478 aa, Bifidobacterium longum
MNNPADAGINLNYLANVRPSSRQLAWQRMEMYAFLHFGMNTMTDREWGLGHEDPALFNPRNVDVDQWMDALVAGGMAGVILTCKHHDGFCLWPSRLTRHTVASSPWREGKGDLVREVSESARRHGLKFGVYLSPWDRTEESYGKGKAYDDFYVGQLTELLTQYGPIFSVWLDGANGEGKNGKTQYYDWDRYYNVIRSLQPDAVISVCGPDVRWAGNEAGHVRDNEWSVVPRRLRSAELTMEKSQQEDDASFATTVSSQDDDLGSREAVAGYGDNVCWYPAEVDTSIRPGWFYHQSEDDKVMSADQLFDLWLSAVGGNSSLLLNIPPSPEGLLAEPDVQSLKGLGRRVSEFREALASVRCEARTSSASAAAAHLVDGNRDTFWRPDADDAAPAITLTLPQPTTINAIVIEEAIEHGQRIEHLRVTGALPDGTERVLGQAGTVGYRRILRFDDVEVSSVTLHVDGSRLAPMISRAAAVRI

SEQ ID NO: 21 - protein, 449 aa, Bifidobacterium longum
MVLFMANPQRPKMYEKFVHDTPEWFKGAGLGIFAHWGSYSVPAWAEPIGALGTFDDPVYWNTHCPYAEWYWNTMSIKGSPAAEHQKEVYGDMPYEDFIDMWKAEAFDPADMADLFARAGARYFVPTTKHHEGITLWKAPDNDGWNTVDRGPHRDLVKEFADAMRDKGLKFGVYYSSGLDWHKEPNMPILGDGEYGPQSEDYARYMYSHVMDLIDEYQPSILWGDIDVPKISEEDNDFSVARLFEHYYDVVPDGVVNDRWGLTHWDFRTVEYEQGKELMGKGMWEMTRGIGYSFGYNQMEDADSYMTGPEAVKLLADVVSMGGNLLLDIGPDAAGRIPELQRQCLEGMADWMDVNSPSIHDVEPVPEASPSGEGDGEPWVRWTGDGKSVYAVVDAAGRVPLRIAADAVDADSAVTLGGSAVAVDADGDVLTADVPASEVAGPQVVHFVRR

SEQ ID NO: 22 - protein, 449 aa, Bifidobacterium longum
MVLFMANPQRPKMYEKFVHDTPEWFKGAGLGIFAHWGSYSVPAWAEPIGALGTFDDPVYWNTHCPYAEWYWNTMSIKGSPAAEHQKEVYGDMPYEDFIDMWKAEAFDPADMADLFARAGARYFVPTTKHHEGITLWKAPDNDGWNTVDRGPHRDLVKEFADAMRDKGLKFGVYYSSGLDWHKEPNMPILGDGEYGPQSEDYARYMYSHVMDLIDEYQPSILWGDIDVPKISEEDNDFSVARLFEHYYDVVPDGVVNDRWGLTHWDFRTVEYEQGKELMGKGMWEMTRGIGYSFGYNQMEDADSYMTGPEAVKLLADVVSMGGNLLLDIGPDAAGRIPELQRQCLEGMADWMDVNSPSIHDVEPVPEASPSGEGDGEPWVRWTGDGKSVYAVVDAAGRVPLRIDAGAVDVDSATILGGGNVVVEADGDMLTVEIPATDVAGPQVVRFARH

SEQ ID NO: 23 - protein, 782 aa, Bifidobacterium longum
MKLTFDGISSHWEEGIPFGNGRMGAVLCSEPDADVLYLNDDTLWSGYPHAETSPLTPEIVAKARQASSRGDYVSATRIIQDATQREKDEQIYEPFGTACIRYSSEAGERKHVKRSLDLARALAGESFRLGAADVHVDAWCSAPDDLLVYEMSSSAPVDASVSVTGTFLKQTRISSGSDSDARQATLVVMGQMPGLNVGSLAHVTDNPWEDERDGIGMAYAGAFSLTVTGGEITVIDDVLQCSGVTGLSLRFRSLSGFKGSAEQPERDMTVLADRLGETIAAWPSDSRAMLDRHVADYRRFFDRVGVRLGPAHDDDEEVPFAEILRSKEDTPHRLETLSEAMFDFGRYLLISSSRPHTQPSNLQGIWNHKDFPNWYSAYTTNINIEMNYWMTGPCALKELIEPLVAMNRELLEPGHDAAGAILGCGGSAVFHNVDIWRRALPANGEPTWAFWPFGQAWMCRNLFDEYLFNQDESYLASIWPIMRDSARFCMDFLSDTEHGLAPAPATSPENYFVVDGETIAVAHTSENTTAIVRNLLDDLIHAAQTMPDLDDGDKALVREAESTRAKLAAVRVGSDGRILEWNDELVEADPHHRHLSHLYELHPGAGITANTPRLEEAARKSLEVRGDDGSGWSIVWRMIMWARLRDAEHAERIIGMFLRPVEADAETDLLGGGVYASGMCAHPPFQIDGNLGFPAALAEMLVQSHDGMVRILPALPEDWHEGSFHGLRARGGLSVDASWTDDAIEYTLRCTKPATITLIVDGTDATQVRLSPDEPFKGLVRR

SEQ ID NO: 24 - protein, 394 aa, Bifidobacterium longum
MTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 25 - protein, 449 aa, Bifidobacterium longum; identical to SEQ ID NO: 21

SEQ ID NO: 26 - protein, 449 aa, Bifidobacterium longum; identical to SEQ ID NO: 22

SEQ ID NO: 27 - protein, 478 aa, Bifidobacterium longum; identical to SEQ ID NO: 20

SEQ ID NO: 28 - protein, 782 aa, Bifidobacterium longum; identical to SEQ ID NO: 23

SEQ ID NO: 29 - protein, 478 aa, Bifidobacterium longum; identical to SEQ ID NO: 20

SEQ ID NO: 30 - protein, 478 aa, synthetic construct
  REGION 1..478: note = Variant W135E; A174F of gi|501574157 (SEQ ID NO: 29)
MNNPADAGINLNYLANVRPSSRQLAWQRMEMYAFLHFGMNTMTDREWGLGHEDPALFNPRNVDVDQWMDALVAGGMAGVILTCKHHDGFCLWPSRLTRHTVASSPWREGKGDLVREVSESARRHGLKFGVYLSPEDRTEESYGKGKAYDDFYVGQLTELLTQYGPIFSVWLDGFNGEGKNGKTQYYDWDRYYNVIRSLQPDAVISVCGPDVRWAGNEAGHVRDNEWSVVPRRLRSAELTMEKSQQEDDASFATTVSSQDDDLGSREAVAGYGDNVCWYPAEVDTSIRPGWFYHQSEDDKVMSADQLFDLWLSAVGGNSSLLLNIPPSPEGLLAEPDVQSLKGLGRRVSEFREALASVRCEARTSSASAAAAHLVDGNRDTFWRPDADDAAPAITLTLPQPTTINAIVIEEAIEHGQRIEHLRVTGALPDGTERVLGQAGTVGYRRILRFDDVEVSSVTLHVDGSRLAPMISRAAAVRI

SEQ ID NO: 31 - protein, 1487 aa, Bifidobacterium bifidum
MLHTASRGCSRSWLRRLTALIAVSALAFVALPNVAVAADPMEYLDVSFGGTFAEDTYTTGGDEVARGSVTKHGSIPTKLDGGGITLAGGTNGVTFTSTASFSASGKVNKGFRAEMEYRTKQTPSNLATLFSAMGNIFVRANGSNLEYGFSTNPSGSTWNDYTKSVTLPSNNVKHIIQLTYLPGADGAASTLQLKVDGVAGETATSAAGELAAVSDSVGNKFGIGYEVNPASGAASRGLAGDVFRARVADSDAPWEILDASQLLHVNFNGTFSGTSYTAASGEQMLGSLVSRSANPSISNSAVTLAGGTAGFDFTPTDFTLGDNEAITTPLVAELRFTPTQTGDNQTLFGAGGNLFLRYESNKLVFGASTKSGDNWTDHKIESAAATGAEHVVSVAYVPNEAGTGAKLVMRVDGGDAQTKDITGLAYLNSGIKGKVGFGNEVHSAALSRGFVGSLSEIRLAKTSANFTTNEFKLVYSQVSCDTSGIKEANTFDVKPAECEAALKTKLSKLRPTEGQADYIDWGQIGFLHYGINTYYNQEWGHGNEDPSRINPTGLDTDQWAKSFADGGFKMIMVTVKHHDGFELYDSRYNTEHDWANTAVAKRTGEKDLFRKIVASAKKYGLKVGIYYSPADSYMERKGVWGNNSARVERTIPTLVKNDDRAGKVASGKLPTFKYKATDYGAYMLNQLYELLTEYGDISEVWFDGAQGNTAGTEHYDYGVFYEMIRRLQPQAIQANAAYDARWVGNEDGWARQTEWSPQAAYNDGVDKVSLKPSQMAPDGKLGTMLSVLSEIRSGAANQLHWYPAEVDAKNRPGWFYHASQSPASVAEVVKYYEQSTGRNSQYLLNVPPSDTGKLADADAAGLKGLGEELARRYGTDLALGKSATVAASANDTAVAAPKLTDGSKLSSDKAVGNTPTYTIDLGSTVAVDAVKISEDVRNAGQQIESATLQGRVNGTWTNLATMTTVGQQRDLRFTSQNIDAIRLVVNSSRGPVRLSRLEVFHTESEIQTGARAYYIDPTAQTAGDGFTKDKPMTSIEQLHDVTVAPGSVIFVKAGTELTGDFAVFGYGTKDEPITVTTYGKSDKATTARFDGMTAGLTLKQALKALGKDDAGWVVADSATAPASRVYVPQDEISVHAQSSQNSGAEAARALDGDSSTSWHSQYSPATSPAPHWVTLDLGKSRENVAYFDYLARIDGNNNGAAKDYEVYVSDDPNDFGAPVASGTLKNVAYTQRIKLTPKNGRYVKFVIKTDYSGSNFGSAAEMNVELLPTAVEEDKVATPQKPTVDDDADTYTIPDIEGVVYKVDGKVLAAGSVVNVGDENVTVTVTAEPADGYRFPDGVTSPVTYELTFTKKGGEKPPTEVNKDKLHATITKAQAIDRSAYTDESLKVLDDKLAAALKVYDDDKVSQDDVNAAEAALSAAIDALKTKPTTPGGEGEKPGEGNKPGDGKKPGDVIAKTGASTMGVVFAALAMVAGAVVTLEAKRKSNR

SEQ ID NO: 32 - protein, 1959 aa, Bifidobacterium bifidum
MKHRAMSSRLMPLVASCATVGMLLAGLPASAVAVGTTRAAASDASSSTTATITPSADTTLQTWTSEKNSSMASKPYIGTLQGPSQGVFGEKFESTDAADTTDLKTGLLTFDLSAYDHAPDSATFEMTYLGYRGNPTATDTDTIKVTPVDTTVCTNNATDCGANVATGATKPKFSIDDSSFVAESKPFEYGTTVYTGDAITVVPANTKKVTVDVTEIVRQQFAEGKKVITLAVGETKKTEVRFASSEGTTSLNGATADMAPKLTVSVSTKDDLKPSADTTLQAWASEKNEKKNTAAYVGALQPEGDYGDFGEKFKSTDVHDVTDAKMGLMTFDLSDYTAAPEHSILTLTYLGYAGADKTATATDKVKVVAVDTSRCTGTAPCDTDNATWANRPDFEVTDTTKTATSHAFAYGSKKYSDGMTVESGNAKKVLLDVSDVIKAEFAKFGTGATEKKITLALGELNKSDMRFGSKEVTSLTGATEAMQPTLSVTKKPKAYTLSIEGPTKVKYQKGEAFDKAGLVVKATSTADGTVKTLTEGNGEDNYTIDTSAFDSASIGVYPITVKYNKDPEIAASFNAYVIASVEDGGDGDTSKDDWLWYKQPASRTDATATAGGNYGNPDNNRWQQTTLPFGNGKIGGTVWGEVSRERVTFNEETLWTGGPGSSTRYNGGNNETKGQNGATLRALNKQLANGAETVNPGNLTGGENAAEQGNYLNWGDIYLDYGFNDTTVTEYRRDLNLSKGKADVTFKHDGVTYTREYFASNPDNVMVARLTASKAGKLNFNVSMPTNTNYSKTGETTTVKGDTLTVKGALGNNGLLYNSQIKVVLDNGEGTLSEGADGASLKVSDAKAVTLYIAAATDYKQKYPSYRTGETAAEVNTRVAKVVQDAANKGYTAVKKAHIADHSAIYDRVKIDLGQSGHSSDGAVATDALLKAYQRGSATTAQKRELETLVYKYGRYLTIGSSRENSQLPSNLQGIWSVTADDNAHGNTPWGSDFHMNVNLQMNYWPTYSANMGELAEPLIEYVEGLVKPGRVTAKVYAGAETTNPETTPIGEGKGYMAHTENTAYGWTAPGQSFSWGWSPAAVPWILQNVYEAYEYSGDPALLDRVYALLKEESHFYVNYMLHKAGSSSGDRLTTGVAYSPEQGPLGTDGNTYESSLVWQMLNDAIEAAKAKGDPDGLVGDTTDCSANNWAKGDNGNFTDANANRSWSCAKSLLKPIEVGDSGQIKEWYFEGALGKKKDGSAISGYQADNQHRHMSHLLGLFPGDLITIDNSEYMEAAKTSLRYRCFKGNVLQSNTGWAIGQRINSWARTGDGNTTYQLVELQLKNAMYANLFDYHAPFQIDGNFGNTSGVDEMLLQSNSTFTDTAGKKYVNYTNILPALPDAWAGGSVSGLVARGNFTVGTTWKNGKATEVKLTSNKGKQAAVKITAGGAQNYEVKNGDTAVNAKVVTNADGASLLVFDTTAGTTYTITKKASANVPVTGVTVTGANTATAGDTVTLTATVTPANATDKSVTWSTSDAAVATVNANGVVTTKKAGKVTITATSNGDKTKFGSIEITVSAATVPVTSVTVAGDAAMTVDGEQTLTATVAPANATDKTVTWKSSDATVATVDANGKVVAKKAGEVTITATAGGVSGTLKITVSDKAPTVIPVQSVTVTGKQELVEGASTTLTATVAPADATDKTVTWKSSDESVATVDKDGVVTAKKAGTVTITATAGGVSGTLDITVTAKPVETVPVTSVEVTVEAGTTVSVGKTLQATATVKPGNATNKKVTWKSSDESIATVDANGVITAKKAGKVVITATSTDGTDKSGSVEITVADETKPTPDHKSVKADTGDVTAGKTGTVTEPKDVAGWKSRSIIKQGKLGKAEIADGTLVYAAGDKTGDDSFVVQYTMADGTVIDVTYSVTVKAAETGKNDGDGKGDGVAKTGAAVGALAGLGLMLLAVGVSVVMIRRKRSA

SEQ ID NO: 33 - protein, 1499 aa, Bifidobacterium bifidum
MIHTASRGRSRSWLRRLTALIAVSALAFVALPNVAVAANPMEYLDVSFGGTFAEDTYTTGGDEVAKGSVTKHGSIPTKLDGGGITLAGGTNGVTFTSTESFSASGKVNKGFRAEMEYRTTQKPSNLATLFSAMGNIFVRANGSNLEYGFSTNPSGSTWNDYKKSVTLPSNNVKHIIQLTYLPGADGAASTLQLKVDGVAGETATSAAGELAAVSDSVGNKFGIGYEVNPASGAASRGLAGDVFRVRVADSDVPWEILDASQLLHVNFNGTFSGTSYTAASGEQMLGSLVSRSANPSISNSAVTLAGGTAGFDFTPTDFTLGDNEAITTPLVAELRFTPTQTGDNQTLFGAGGNLFLRYESNKLVFGASTKSGDNWTDHKIESAAATGAEHVVSVAYVPNEAGTGAKLVMRVDGGDAQTKNITGLAYLNSNIKGKVGFGNDVHSAALSRGFVGSLSEIRLAKTSANFTTNEFKLVYSQVSCDTSGIKEANTFDVKPAECEAALKTKLSKLRPTEGQADYIDWGQIGFLHYGINTYYNQEWGHGNEDPSRIDPTGLDTDQWAKSFADGGFKMIMVTVKHHDGFELYDSRYNTEHDWANTAVAKRTGEKDLFRKIVASAKKYGLKVGIYYSPADSYMEKKGVWGNNSARVERTIPTLVENDDRAGKVASGKLPTFKYKATDYGAYMLNQLYELLTEYGDISEVWFDGAQGNTAGTEHYDYGVFYKMIRQLQPQAIQANAAYDARWVGNEDGWARQTEWSPQAAYNDGVDKVSLKPGQMAADGTLGTMSSVLSEIRSGAANQLHWYPAEVDAKNRPGWFYHANQSPASVAQVVKYYEQSTGRNSQYLLNVPPSDTGKLADADAAGLKGLGEELARRYGTDLALGKSATVAASANDTAVAAPKLTDGSKLSSDKAVGNTPTYTIDLGSTVAVDAVKISEDVRNAGQQIESATLQGRVNGTWTNLATMTTVGQQRDLRFTSQNIDAIRLVVNSSRGPVRLSRLEVFHTESEIQTGARAYYIDPTAQTAGDGFTKDKPMTSIEQLHDVTVAPGSVIFVKAGTELTGDFAVFGYGTKDEPITVTTYGKSDKATTARFDGMTAGLTLKQALKALGKDDAGWVVADSATAPASRVYVPQDEISVHAQSSQNSGAEAARALDGDSSTSWHSQYSPATSPAPHWVTLDLGKSRENVAYFDYLARIDGNNNGAAKDYEVYVSDDSNDFGAPVASGTLKNVAYTQRIKLTPKNGRYVKFVIKTDYSGSNFGSAAEMNVELLPTAVEEDKVATPQKPTVDDDADTYTIPDIEGVVYKVDGKVLAAGSVVNVGDEDVTVTVTAEPADGYRFPDGVTSPVTYELTFTKKGGEKPPTEVNKDKLHATITKAQAIDRSAYTDESLKVLDDKLAAALKVYDDDKVSQDDVDAAEAALSAAIDALKTKPTTPGGEGEKPGEGNKPGEGNKPGEGNKPGDGKKPGDVIAKTGASTMGVVFAALAMVAGAVVTLEAKRKSNR

SEQ ID NO: 34 - protein, 478 aa, Bifidobacterium longum
MSNPTNDGINLNYLANVRPSSRQLVWQRMEMYAFIHFGMNTMTDREWGLGHEDPALFDPQNVDVEQWMDALVAGGMTGVILTCKHHDGFCLWPSRYTQHTIAASPWRDGKGDLVREVSESARRHGLKFGVYLSPWDRTEESYGKGKAYDDFYVGQLTELLTQYGPIFSVWLDGANGEGKNGKTQYYDWDRYYNVIRSLQPNAVISVCGPDVRWAGNEAGHVRDNEWSVVPRRLRSAELTMENSQQEDDASFASTVRSQDDDLGSREAVSGYGDDVCWYPAEVDTSIRPGWFYHKYEDDKVMSADQLFDLWLSAVGGNSSLLLNIPPSPEGLFAEPDVESLKGLGSRINEFRKALASSCCEVKTSSANEAAMRLIDGNQDTYWSPSTDDVAPAVTLTFPQLTTINAVVVEEAIECGQRIEHMRVTGVLSDGTECVLGQFGTVGYRRILRFDDVEVSSVTLHVDDSRFTPMISRAAAVRI

SEQ ID NO: 35 - protein, 497 aa, Bifidobacterium dentium
MIDLSTVEAVIASGPYADTWESLSQAPAPAWFTDAKFGIFTHWGLYSVPEYRNEWYSRNMYIEGYPEFEHHVNTYGSQNKFGYRDFIPMFTAERFDANEWLDLFAAAGARYYFPVSEHHDGYQMYRSELSHWNTFETGPHRDVLGELRDATHAHGLHFATSNHRAEHWWFMGHGKDFDSDIKEPLQKGDFYWPAMPEPNEFDIHSTPTPTAEYLEDWLLRVCEIIDDYRPELLYFDWWVQHESFRPYMRKLAAFYYNRGVEWGRNVSICYKYDGLAWGAGIVDVERGGFADATPFVWQTDTAIARNSWCYTNSLEYKTLAELIVALVDAVSKNGNLLLNVGPRADGSIADHDRELLLGIGDWLRTNGEGIYGTRPWRLTQEGPTKQADGMFADQKPTEWTAADWRFTTKNGAIYAFCLHPNDIADSCETSCETLAMSTFAAYHDGVKPPFNGIVESVEQLGAGPVRFKRGDEALEISPLPREPDQQGLPIGFKITVG

SEQ ID NO: 36 - protein, 378 aa, Lactobacillus casei
MTKPSAVNIHRTNPPDWFKQADFGIIIHWGPYSVPAYAPVNIPDYGTLIKTKSLGYLFEHQPYAEWYGNSMLLPSSPVAAYHRTHYGNTSYADFAKTFKQTAQNVDVQAWAAAFANAGAKYVVIVTKHHDGFVMFDPHTKNPYEPDYHLNFDFVGELAQAVRAHGMRFGTYYSSLLDWTFPHLPIKDYGSFLLGNDKSQTYKDYVWHQWHELIDRYHPDVLWNDIGYPDDHRLETLFKYYYQQVPEGLVNDRWQQFPDWMRTSWIRPIFNLVAAQVIKRDQHHSNDLSEVKYYDYRTFEYRTDWPQTNRYFEMTRGMDKSFGYNRLSRPEDYITADDIKQLIAELRSKRGRLLLNVGPDMNGQIPSAQLSILQQLSDR

SEQ ID NO: 37 - protein, 414 aa, Lactobacillus casei
MTEPLPRIQHYEDLGLGLFIHWGLYSQMAVGEWTELIHHRNQHDYEQLIKTFTAAQFDAKKIAHAAKAVGAKYIVLTTKHHEGFFLYDTKGLSDFDVMHAPARRDLIAEFVAACREEDLLPFFYMATYDWHTPLYDDDFPAYLTYLQKSVEVLCRNYGPVGGFWFDGNWNKKDADWHLPELYGMIRHYQPNAIIVNNTGLKNRGQVSDPEIDVVTYERRTPDEIYHGAPNEKYVAGEISITLNQHWGIAANDLNYKSPAEVIETVAHARHIGANILVNIGLTGTGAIPAAAQTYMHLLGRWTAMAAPVLYKGRPVPVTSAHGTRDFVLHTSKHDFLCILDLQVVGNDNVVLGGEGVNPRSFVGIGQPIQRIHWLDNDEVLSFTQDLDKKVLTVDATGYPYGSDWVVRIAQIDYE

SEQ ID NO: 38 - protein, 344 aa, Lactobacillus casei
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPKNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 39 - protein, 478 aa, synthetic construct
  REGION 1..478: note = Variant S168E; A174H; E413R of gi|501574157 (SEQ ID NO: 29)
MNNPADAGINLNYLANVRPSSRQLAWQRMEMYAFLHFGMNTMTDREWGLGHEDPALFNPRNVDVDQWMDALVAGGMAGVILTCKHHDGFCLWPSRLTRHTVASSPWREGKGDLVREVSESARRHGLKFGVYLSPWDRTEESYGKGKAYDDFYVGQLTELLTQYGPIFEVWLDGHNGEGKNGKTQYYDWDRYYNVIRSLQPDAVISVCGPDVRWAGNEAGHVRDNEWSVVPRRLRSAELTMEKSQQEDDASFATTVSSQDDDLGSREAVAGYGDNVCWYPAEVDTSIRPGWFYHQSEDDKVMSADQLFDLWLSAVGGNSSLLLNIPPSPEGLLAEPDVQSLKGLGRRVSEFREALASVRCEARTSSASAAAAHLVDGNRDTFWRPDADDAAPAITLTLPQPTTINAIVIEEAIRHGQRIEHLRVTGALPDGTERVLGQAGTVGYRRILRFDDVEVSSVTLHVDGSRLAPMISRAAAVRI

SEQ ID NO: 40 - protein, 511 aa, Photobacterium leiognathi
MKRIFCLVSAILLSACNDNQNTVDVVVSTVNDNVIENNTYQVKPIDTPTTFDSYSWIQTCGTPILKDDEKYSLSFDFVAPELDQDEKFCFEFTGDVDGKRYVTQTNLTVVAPTLEVYVDHASLPSLQQLMKIIQQKNEYSQNERFISWGRIGLTEDNAEKLNAHIYPLAGNNTSQELVDAVIDYADSKNRLNLELNTNTAHSFPNLAPILRIISSKSNILISNINLYDDGSAEYVNLYNWKDTEDKSVKLSDSFLVLKDYFNGISSEKPSGIYGRYNWHQLYNTSYYFLRKDYLTVEPQLHDLREYLGGSLKQMSWDGFSQLSKGDKELFLNIVGFDQEKLQQEYQQSELPNFVFTGTTTWAGGETKEYYAQQQVNVVNNAINETSPYYLGREHDLFFKGHPRGGIINDIILGSFNNMIDIPAKVSFEVLMMTGMLPDTVGGIASSLYFSIPAEKVSFIVFTSSDTITDREDALKSPLVQVMMTLGIVKEKDVLFWSDLPDCSSGVCIAQY

SEQ ID NO: 41 - protein, 497 aa, synthetic construct
  REGION 1..497: note = N-terminal truncated variant of gi|267844791 (SEQ ID NO: 40)
MCNDNQNTVDVVVSTVNDNVIENNTYQVKPIDTPTTFDSYSWIQTCGTPILKDDEKYSLSFDFVAPELDQDEKFCFEFTGDVDGKRYVTQTNLTVVAPTLEVYVDHASLPSLQQLMKIIQQKNEYSQNERFISWGRIGLTEDNAEKLNAHIYPLAGNNTSQELVDAVIDYADSKNRLNLELNTNTAHSFPNLAPILRIISSKSNILISNINLYDDGSYEYVRLYNWKDTEDKSVKLSDSFLVLKDYFNGISSEKPSGIYGRYNWHQLYNTSYYFLRKDYLTVEPQLHDLREYLGGSLKQMSWDGFSQLSKGDKELFLNIVGFDQEKLQQEYQQSELPNFVFTGTTTWASGETKEYYAQQQVNVVNNAINETSPYYLGREHDLFFKGHPRGGIINDIILGSFNNMIDIPAKVPFEVLMMTGMLPDTVGGIASSLYFSIPAEKVSFIVFTSSKTITDREDALKSPLVQVMMTLGIVKEKDVLFWSDLPDCSSGVCIAQY

SEQ ID NO: 42 - DNA, 300 bp, synthetic construct
  source 1..300: mol_type = other DNA
  misc_feature 1..300: note = Promoter sequence
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 43 - protein, 483 aa, Photobacterium damselae
MCNSDNTSLKETVSSNSADVVETETYQLTPIDAPSSFLSHSWEQTCGTPILNESDKQAISFDFVAPELKQDEKYCFTFKGITGDHRYITNTTLTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 44 - DNA, 2818 bp, Campylobacter jejuni
  source 1..2818: mol_type = other DNA
atgaaagaaataaaaatacaaaatataatcataagtgaagaaaaagcacccttagtcgtacctgaaataggcattaatcataatggcagtttagaactagctaaaattatggtagatgcagcctttagcgcaggtgctaagattataaagcatcaaactcatattgttgaagatgagatgagtaaggccgctaaaaaagtaattcctggtaatgcaaaaataagcatttatgagattatgcaaaaatgtgctttggattataaagatgagctagcacttaaagaatacacagaaaaattaggtcttgtttatcttagcacacctttttctcgtgcaggtgcgaaccgcttagaagatatgggagttagtgcttttaagattggttcaggtgagtgtaataattatccgcttattaaacacatagcagcctttaaaaagcctatgatagttagcacaggaatgaatagtattgaaagtataaaaccaactgtaaaaatcttattagacaatgaaattccttttgttttaatgcacacgaccaatctttacccaaccccgcataatcttgtaagattaaacgctatgcttgagttaaaaaaagaattttcttgtatggtaggcttaagcgaccacacaacagataatcttgcgtgtttaggtgcagttgtacttggagcttgtgtgcttgaaagacattttactgatagtatgcatagaagtggccctgatatagtttgttctatggatacaaaggctttaaaagagctaattatacaaagtgagcaaatggctataataagaggaaataatgaaagtaaaaaagcggctaaacaagaacaagttacaattgattttgcctttgcaagtgtagttagcattaaagatattaaaaaaggcgaagttttatctatggataatatttgggttaaaagacctggacttggtggaattagtgcggctgaatttgaaaatattttaggcaaaaaagcattaagagatatagaaaatgatgctcagttaagctatgaggattttgcgtgaaaaaaatcctttttataacaggctctagggctgattattctaagattaaatctttaatgtacagggtgcaaaactcaagcgaatttgaactttacatctttgcaacaggaatgcacttaagtaaaaattttggctatacagttaaagaactttataaaaatggctttaaaaatatttatgaatttataaattatgataaatattatcaaactgataaggctttagctactacaattgatggattttcaaggtatgcaaatgagctaaaacctgatttaatcgtagtacatggagatagaattgagcctttagcagcagctattgttggagcattaaataatatcttagtagcgcatattgaaggcggagagatttcaggaactattgacgatagcttacgccacgctatatcaaaactagctcatattcatttagtaaatgatgagtttgcaaaaaggcgtttaatgcagcttggagaagatgaaaaatctatttttatcataggttcgcctgatttagaacttttaaacgataataaaatttcacttagcgaagcaaaaaaatattatgatataaattatgaaaactacgctttgcttatgtttcatcctgttacaactgaaattactagcattaaaaatcaagcagacaatttagtaaaagcactgatacaaagtaataaaaattatattgttatttatccaaataatgatttaggttttgaattaatcttgcaaagctatgaagagtttaaaaataaccctagatttaagctttttccatcgcttagatttgagtattttataactttgttaaaaaatgctgattttataataggtaattcaagttgtattttaaaagaggccttatacttaaaaacagcagggattttagttggctcaagacaaaatggaagacttggcaatgaaaatacactaaaagttaatgcaaatagtgatgaaatactaaaagctattaacactattcataaaaaacaagatttatttagcgctaagttagagattttagatagctcaaaattattttttgaatatttacaaagcggagatttttttaaactcagcacacaaaaagtttttaaggatataaaatgagcttagcaataatccctgctcgtggtggctcaaagggtattaaaaataaaaatttggttttattaaacaataaacctttaatttactacacgatcaaagctgcactaaatgctaaaagcattagtaaagttgttgtaagcagtgatagtgatgaaattttaaattatgcaaaaagtcaaaatgttgatattttaaaacgcccaattagccttgcacaagatgataccacaagcgataaagtgctgttacatgctctaaaattttataaagattatgaagatgtagtttttttacaacccacttcaccgctaagaacaaatattcatattaatgaagcttttaatctttataaaaatagcaatgcaaatgccctaattagcgtaagcgaatgtgataataaaattctaaaagcctttgtttgtaatgattgtggcgatttagcagggatttgtaatgatgaatatccttttatgccaaggcaaaaattgcctaaaacttatatgagcaatggtgcaatttatattttaaagataaaagaatttttaaacaatcctagctttttacaaagcaaaaccaagcattttttaatggacgaaagctcaagtttagatattgactgtttggaggatttaaaaaaggttgaacagatatggaaaaaataa

SEQ ID NO: 45 - protein, 332 aa, Neisseria meningitidis
MQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 46 - protein, 273 aa, Helicobacter pylori
MRVFAISLNQKVCDTFGLVFRDTTTLLNSINATHHQAQIFDAIYSKTFEGGLHPLVKKHLHPYFITQNIKDMGITTNLISEVSKFYYALKYHAKFMSLGELGCYASHYSLWEKCIELNEAICILEDDITLKEDFKEGLDFLEKHIQELGYIRLMHLLYDASVKSEPLSHKNHEIQERVGIIKAYSEGVGTQGYVITPKIAKVFLKCSRKWVVPVDTIMDATFIHGVKNLVLQPFVIADDEQISTIARKEEPYSPKIALMRELHFKYLKYWQFV

SEQ ID NO: 47 - protein, 505 aa, Klebsiella pneumoniae
MYKKRKLAILIALLTGTAAAHGQTDLNSIEARLAALEKRLQDAETRASTAESRAASAEQKVQQLTQQQQQTQATTQQVARRTTQLEEKAERPGGFEFHGYARSGVIMNDSAASTKSGAYMTPAGETGGAIGRLGNQADTYVEMNLEHKQTLDNGATTRFKVMVADGQTTYNDWTASSSDLNVRQAFVELGNLPTFEGPFKGSTLWAGKRFDRDNFDIHWIDSDVVFLAGTGGGIYDVKWNDSLRSNFSLYGRNFGDIADSSNSVQNYIVSMNNFAGPVQMMVSGMRAKDNDDRQDANGNLVKGDAANTGVHALLGLHNESFYGLRDGTSKTALLYGHGLGAEVKGIGSDGALRPGANTWRFASYGTTPLSDRWFIAPAVLAQSSKDRYVDGDSYQWATLNLRLIQEVTQNFALAWEGSYQYMDLQPEGYNDRHAVNGSFYKLTFAPTFKVGSIGDFFSRPEIRFYTSWMDWSKKLDNYANDDALGSNGFKSGGEWSFGMQMETWF

SEQ ID NO: 48 - protein, 456 aa, Klebsiella pneumoniae
MDFEQISRSLLPLLGGKENIASAAHCATRLRLVLVDDALADQQAIGKIDGVKGCFRNAGQMQIIFGTGVVNKVYAAFIQAAGISESSKSEAADLAAKKLNPFQRIARLLSNIFVPIIPAIVASGLLMGLLGMVKTYGWVDPSNALYIMLDMCSSAAFIILPILIGFTAAREFGGNPYLGATLGGILTHPALTNAWGVAAGFHTMNFFGIEVAMIGYQGTVFPVLLAVWFMSMVEKRLRRVIPDALDLILTPFLTVIISGFIALLLIGPAGRALGDGISFILSTLISHAGWLAGLLFGGLYSVIVITGIHHSFHAIEAGLLGNPSIGVNFLLPIWAMANVAQGGACFAVWFKTKDAKIKAITLPSAFSAMLGITEAAIFGINLRFVKPFIAALVGGAAGGAWVVSMHVYMTAVGLTAIPGMAIVQASSLLNYIIGMAIAFAVAFALSLTLKYKTDAE

SEQ ID NO: 49 - protein, 466 aa, Salmonella enterica
MSLPSRLPAILQAVMQGQPRALADSHYPRWHHAPVTGLMNDPNGFIEFAGRYHLFYQWNPLACDHTFKCWAHWSSIDLLHWQHEPIALMPDEEYDRNGCYSGSAVDNNGTLTLCYTGNVKFAEGGRTAWQCLATENADGTFRKIGPVLPLPEGYTGHVRDPKVWRHEDLWYMVLGAQDRQKRGKVLLFSSADLHQWTSMGEIAGHGINGLDDVGYMWECPDLFPLGDQHILICCPQGIAREEECYLNTYPAVWMAGEFDYAAGAFRHGELHELDAGFEFYAPQTMLTSDGRRLLVGWMGVPEGEEMLQPTLNNGWIHQMTCLRELEFINGQLYQRPLRELSALRGEANGWSGNALPLAPMEIDLQTRGGDMLSLDFGGVLTLECDASGLRLARRSLASDEMHYRYWRGNVRSLRVFIDQSSVEIFINGGEGVMSSRYFPACSGQLTFSGITPDAFCYWPLRTCMVE

SEQ ID NO: 50 - protein, 334 aa, Salmonella enterica
MKTKRVTIKDIAEQAGVSKATASLVLNGRGKELRVAQETRERVLSIARKHHYQPSIHARSLRNNRSHTIGLVVPEITNHGFAVFAHELEMLCREAGVQLLISCTDENPGQESVVVNNMIARQVDGMIVASCMHNDADYLKLSQQLPVVLFDRCPNESALPLVMTDSITPTAELISRIAPQHSDEFWFLGGQARLSPSRDRLTGFTQGLAQAGIALRPEWVINGNYHPSSGYEMFAALCARLGRPPKALFTAACGLLEGVLRYMSQHHLLDSDIHLTSFDDHYLYDSLSLRIDTVQQDNRQLAWHCYDLISQLIEGDTPETLQRYLPATLQFRHQ

SEQ ID NO: 51 - protein, 392 aa, Pantoea vagans
MKSLLTRKRRINPVFLAFMAASFMIGVAGALQAPTLSLFLTREVQARPLWVGLFFTVNAIAGIVVSMLVAKRSDSRGDRRTLILFCCAMAFCNALLFAFTRHYLTLITLGVLLSALASVSMPQIFALAREYADQSAREAVMFSSVMRAQLSLAWVIGPPLSFALALNFGFVTLFLVAAALFLVCILLIKFTLPSVPRAEPLMRSGGMPLSGWRDRDVRLLFIASVTMWTCNTMYIIDMPLYISVTLGLPEKLAGLLMGTAAGLEIPVMLLAGHYAKRVGKRNLMLIAVAAGVLFYAGLAMFASQTALMALQLFNAVFIGIIAGIGMLWFQDLMPGRPGAATTMFTNSISTGMILAGVIQGTLSERFGHIAVYWLALGLAVAAFAMSARVKNV

SEQ ID NO: 52 - skipped

SEQ ID NO: 53 - DNA, 300 bp, synthetic construct
  source 1..300: mol_type = other DNA
  misc_feature 1..300: note = Promoter sequence
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 54 - DNA, 152 bp, Klebsiella pneumoniae
  source 1..152: mol_type = other DNA
ggttaacggcccactttgctggcgacatcacaattcttaaaccggtttagcaatttttattttcaccgcgttaccgacatgtttaccatatcaactaaaccggtttagcaaacattagcacactcactgatttacctttggatgtcaccaac

SEQ ID NO: 55 - DNA, 107 bp, Escherichia coli
  source 1..107: mol_type = other DNA
tgtgagttagctcactcattaggcaccccaggctttacactttatgcttccggctcgtatgttgtgtggaattgtgagcggataacaatttcacacaggaaacagct

SEQ ID NO: 56 - protein, 439 aa, synthetic construct
MISVYIISLKESQRRLDTEKLVLESNEKFKGRCVFQIFDAISPKHEDFEKFVQELYDSSSLLKSDWFHSDYCYQELLPQEFGCYLSHYLLWKECVKLNQPVVILEDDVALESNFMQALEDCLKSPFDFVRLYGHYWGGHKTNLCALPVYTETEEAEASIEKTPIENYEVTSPPPPNPTRDTQQDFITETQQDPKELSEPCKIAPQKISFNQVVFKKIKRKLNRFIGSILARTEVYKNIVAKYDDLTTKYDDLTTKYDDLTTKYDDLTTKYDDLNKNIAEKYDELMGKYESLLAKEVNIKETFWESRADSEKEALFLDHFYLTSVYVATTAGYYLTPKGAKTFIEATERFKIIEPVDMFINNPTYHDIANFTYVPCPVSLNKHAFNSTIQNAKKPDISLKPPKKSYFDNLFYHKFNARKCLKAFNKYSKQYAPLKTPKEV

SEQ ID NO: 57 - protein, 393 aa, Yersinia bercovieri
MKSALTFSRRINPVFLAFFVVAFLSGIAGALQAPTLSLFLSTEVKVRPLWVGLFYTVNAIAGITVSFILAKRSDSRGDRRKLIMVCYLMAVGNCLLFAFNRDYLTLITAGVLLASVANTAMPQIFALAREYADSSAREVVMFSSIMRAQLSLAWVIGPPLSFMLALNYGFTLMFSIAAGIFVLSALVVWFILPSVPRAEPVVDAPVVVQGSLFADKNVLLLFIASMLMWTCNTMYIIDMPLYITASLGLPERLAGLLMGTAAGLEIPIMLLAGYSVRYFGKRKIMLFAVLAGVLFYTGLVLFKFKTALMLLQIFNAIFIGIVAGIGMLYFQDLMPGRAGAATTLFTNSISTGVILAGVLQGGLTETWGHDSVYVMAMVLSILALIICARVREA

SEQ ID NO: 58 - protein, 342 aa, Neisseria meningitidis
MERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 59 - protein, 478 aa, synthetic construct
MNNPADAGINLNYLANVRPSSRQLAWQRMEMYAFLHFGMNTMTDREWGLGHEDPALFNPRNVDVDQWMDALVAGGMAGVILTCKHHDGFCLWPSRLTRHTVASSPWREGKGDLVREVSESARRHGLKFGVYLSPFDRTEESYGKGKAYDDFYVGQLTELLTQYGPIFSVWLDGNNGEGKNGKTQYYDWDRYYNVIRSLQPDAVISVCGPDVRWAGNEAGHVRDNEWSVVPRRLRSAELTMEKSQQEDDASFATTVSSQDDDLGSREAVAGYGDAVCWYPAEVDTSIRPGWFYHQSEDDKVMSADQLFDLWLSAVGGNSSLLLNIPPSPEGLLAEPDVQSLKGLGRRVSEFREALASVRCEARTSSASAAAAHLVDGNRDTFWRPDADDAAPAITLTLPQPTTINAIVIEEAIRHGQRIEHLRVTGALPDGTERVLGQAGTVGYRRILRFDDVEVSSVTLHVDGSRLAPMISRAAAVRI

SEQ ID NO: 60 - protein, 642 aa, synthetic construct
MLAPGSSRVELFKRQSSKVPFEKDGKVTERVVHSFRLPALVNVDGVMVAIADARYETSNDNSLIDTVAKYSVDDGETWETQIAIKNSRASSVSRVVDPTVIVKGNKLYVLVGSYNSSRSYWTSHGDARDWDILLAVGEVTKSTAGGKITASIKWGSPVSLKEFFPAEMEGMHTNQFLGGAGVAIVASNGNLVYPVQVTNKKKQVFSKIFYSEDEGKTWKFGKGRSAFGCSEPVALEWEGKLIINTRVDYRRRLVYESSDMGNTWLEAVGTLSRVWGPSPKSNQPGSQSSFTAVTIEGMRVMLFTHPLNFKGRWLRDRLNLWLTDNQRIYNVGQVSIGDENSAYSSVLYKDDKLYCLHEINSNEVYSLVFARLVGELRIIKSVLQSWKNWDSHLSSICTPADPAASSSERGCGPAVTTVGLVGFLSHSATKTEWEDAYRCVNASTANAERVPNGLKFAGVGGGALWPVSQQGQNQRYHFANHAFTLVASVTIHEVPSVASPLLGASLDSSGGKKLLGLSYDERHQWQPIYGSTPVTPTGSWEMGKRYHVVLTMANKIGSVYIDGEPLEGSGQTVVPDERTPDISHFYVGGYKRSDMPTISHVTVNNVLLYNRQLNAEEIRTLFLSQDLIGTEAHMDSSSDTSA

SEQ ID NO: 61 - protein, 22 aa, Erwinia carotovora
MKYLLPTAAAGLLLLAAQPAMA

SEQ ID NO: 62 - protein, 21 aa, Escherichia coli
MKKTAIAIAVALAGFATVAQA

SEQ ID NO: 63 - protein, 19 aa, Escherichia coli
MKKIWLALAGLVLAFSASA

SEQ ID NO: 64 - protein, 25 aa, Escherichia coli
MMITLRKLPLAVAVAAGVMSAQAMA

SEQ ID NO: 65 - protein, 21 aa, Escherichia coli
MKQSTIALALLPLLFTPVTKA

SEQ ID NO: 66 - protein, 26 aa, Escherichia coli
MKIKTGARILALSALTTMMFSASALA

SEQ ID NO: 67 - protein, 23 aa, Escherichia coli
MNKKVLTLSAVMASMLFGAAAHA

SEQ ID NO: 68 - protein, 20 aa, Escherichia coli
MKKGFMLFTLLAAFSGFAQA

SEQ ID NO: 69 - protein, 1599 aa, Bifidobacterium longum
MTSRQGRQAIAATAAMGVAVALALPTAAFAQSATQGKETATTTSSGTTYYVSSAHGDDANAGTSENAPWKSLTKVNDIASDLGPGDSVLLEYGSEFNDQYLHIKDTAGNADAPITISAYGDADEGKPVIASNGVKGSQWEQDYRANVGNHKNKGTVSTTLLLKDVSYITVSNLEITNDDADVYDPIDTWKWTDTPDSDGTKLDRSASRMDRTGVAGIAENGATMSNVTLDNLYIHDVDGNIYNKHMANGGIYFMAHYPMENTSAETDVWLREHVSRFDHVTIRNSTVKDVDRWGIAVGYTAYLNYIDANYGDGSIDDALIAKYGSTNVRIENNYVKGAGGDAITLMYCDRPVIEHNVGDSVSKHINTQDYTQPGSYGGRVAAGIWPWRCKDPVFQYNEMYNNLNAEHGNGDGQAWDADYGDGTLYQYNYSYGNSFASLMICNWYAVNTTFRYNISQNDRQGVFDLPSNGPGNHIYNNTVYVDADSQVLTKRSNSQSLFENNIFINATNTKKTETWNRGSQNGGQTYDNNMYVNYANKPTSDANAIEADDVSAVLAGAGSAPTSALKSGAEHARTGEKAAFDGYRPVAGSKAINAGKVVSDLNDYAVENDFLGNAVKGRPDLGAVESDVVSVTMASSKYETGTETDSGTGDKTKVIHVTFTDKNPVTVKELLSNVSADKGVDKAVYRVADAKSGKSADARSAESEPNMLDRLLSLLPGSDRNAKDDETKLADSEPVRDGDILRFSAEGTDETDEYTIRQRITWDWVADYEQGVADFDWKAQRRTSAGGEWTTISAYDGSWPNTVYDQYYGVGVNGTLAELSGDRKQTHGLLIDKPGDGLPTAMAWKAPESGTVMLSLKTFADKIAEPYLRQNADNAGKKVTLSLMRNDETLCSADDLSVYQKSSEQFAQCLAEHGSIDVQEGDWIRIVADAETGVKAPSLHISPVITYEDKAPAAPKQNVRYDVSYAATDAVVGTQSAVAAAFTADGGEADAPDGVAFAFKDGGDEGEASPVIDASTGAVTFTPAAGQYGATVTRTVVVTYADGSSDETTVTFRVAQSHAQRLNVLYPTVRGDAGTDLKRTPKFTLKADGAAASVPEGTTFALGANAPAGASVDMANGTVTLNSGVGGTVTVPVTVTFADDGASVSSTARFEVTAPAALGSSELETATVDGVNVVYAPFSADSPMTVAQLLAKVTAEPSGADKGVYRDGVRLEAGAELAENDVLRFSAKGSTVSDDYVVKSKTTWDWVNDFQVRVQGPIWYGQRQTEADGVWSDIADFDATYPNWMYETYYGPGVDYANHSLPTDRSAIHGLISDSPASAGGSAMAWKAPKAGTVKVSIREDEPYLRQDGSNGKALTLRLMHDDKVVCFADLTVSKQRSEEFANCVADKGEIAVEAGDWIRVTATSASGMNKPSAHISPVIAYMAASTPGPEPVPVDKSTLKATVEEALGLAESDYTDESWAALVAARDAAQTVLDDDAATAEQVETAQNALRDAIDGLEKKPVDPDPNPKPDPNPDPDPTPDPDPDPGPDTKPGDGSGNGSGTGNGSGSGNGSTGSGSDGATTGGKLTATGADVAGAAAMVALTAAAGIGLAAAARRRR

(19) United States
(12) Patent Application Publication
(10) Pub. No.: US 2026/0062726 A1
(43) Pub. Date: Mar. 5, 2026

(54) HYBRID METHOD FOR PRODUCING COMPLEX HMOS

(71) Applicant: DSM IP Assets B.V., Maastricht (NL)

(72) Inventors: Getachew S. MOLLA, Hørsholm (DK); Ted JOHANSON, Hørsholm (DK); Niels BANKE, Hørsholm (DK)

(21) Appl. No.: 19/105,083

(22) PCT Filed: Aug. 25, 2023
(86) PCT No.: PCT/EP2023/073430

§ 371 Date: Feb. 20, 2025

(30) Foreign Application Priority Data
Aug. 25, 2022 (EP) 22192211.5
Nov. 25, 2022 (EP) 22209675.2

(51) Int. Cl. C12P 19/04; C12N 9/24; C12P 39/00

(52) U.S. Cl. CPC C12P 19/04; C12N 9/2402; C12P 39/00; C12Y 302/01018

(57) ABSTRACT

The present disclosure relates to a method for producing oligosaccharides where the method combines a fermentation process and an enzymatic reaction in the medium of the fermentation, thereby enabling the formation of the oligosaccharide in the fermentation medium of an ongoing fermentation. The method is in particular suitable for the generation of complex oligosaccharides.

### FIELD OF INVENTION

The present disclosure relates to a method for producing complex oligosaccharides, such as complex fucosylated and/or sialylated HMOs or neutral HMOs of at least six monosaccharides. The hybrid method combines a fermentation and an enzymatic reaction in the medium of the fermentation allowing formation of the complex oligosaccharide in the medium of an ongoing fermentation. Also described herein are HMO mixtures derived from the hybrid process as well as uses of such mixtures.

### BACKGROUND

Production of more complex human milk oligosaccharides (HMOs) has been pursued via chemical synthetic routes, enzymatic routes and in vivo fermentation approaches. For industrial purposes the chemical routes are too complex and expensive.

The enzymatic synthesis of HMOs relies on the use of a donor oligosaccharide (HMO) and an acceptor oligosaccharide (HMO) which are catalyzed by an enzyme with transglycosidase activity to produce a third oligosaccharide (complex HMO), however due to the nature of enzymatic reactions the HMOs produced by this route will always be a mixture of the donor, the acceptor and the third oligosaccharide (HMO) as well as a side-product released moiety of the donor substrate (the leaving group e.g. lactose) due to the equilibrium of the enzymatic reaction (see for example WO2012/156897, WO2012/156898 and WO2016/063262). Furthermore, the enzymatic process utilizes separately produced and purified donor and acceptor substrates which increases the cost of the process. Additionally, in cases where side-product released from the donor substrate is lactose, extensive purification is required to remove this large amount of lactose which is undesired.

Bioproduction systems using in vivo fermentation of the HMOs is currently the preferred mode of production for the smaller fucosylated and sialylated and neutral core HMOs (for review see Bych et al 2019, Current Opinion in Biotechnology 56:130-137). However, with more complex HMOs the fermentation route may face challenges in terms of exporting the HMOs from the cell into the medium, which is necessary to achieve high yields in industrial scale.

Since lactose is used as the initial substrate in many bioproduction processes there is often an excess of lactose at the end of the fermentation which may interfere with the subsequent purification of the desired oligosaccharide product. WO2015/036138 describes the use of suitable glycosidase, exemplified by beta-galactosidase, in the culture medium for the degradation of excess lactose.

### OBJECTIVE OF THE INVENTION

The present disclosure has identified a hybrid process, suitable for producing one or more oligosaccharides, such as HMOs. The process is in particular suitable for producing complex fucosylated and/or sialylated HMOs. The hybrid process is a combination of a fermentation and an enzymatic reaction of the fermentation product (first oligosaccharide) with a second oligosaccharide or disaccharide in the medium of the fermentation, resulting in the production of the desired third, more complex, oligosaccharide in the culture medium of the fermentation. Some of the benefits of this process is that none or at least only one of the starting oligosaccharides (the second oligosaccharide) needs to be in a pure form, reducing cost of the process, and the side-product (e.g. lactose) produced during the enzymatic step is in situ recycled by the genetically modified microbial strain(s) as a starting material for producing the first oligosaccharide and potentially second oligosaccharide thereby reducing the final side-product (e.g., lactose) levels from the enzymatic process at the end of the process. In embodiments where the process only comprises one genetically modified cell the feeding of lactose during the entire fermentation of the first oligosaccharide can be signifyingly reduced or omitted entirely. The recycling of the lactose also shifts the equilibrium of the enzymatic process towards the desired third oligosaccharide (circumventing the equilibrium barrier) to an almost full conversion of either the first or the second substrate (donor or acceptor) oligosaccharide. The desired third oligosaccharide can therefore be produced in higher ratios to the first and/or second oligosaccharides than in the conventional enzymatic process. Furthermore, since the generation of the desired oligosaccharide is occurring in the medium of the fermentation and not inside the genetically modified microbial strain the challenge of exporting a complex/large oligosaccharide out of the cell is solved.

### SUMMARY

The current application relates to a hybrid method for producing a oligosaccharide, wherein the method combines a fermentation process producing a donor substrate (oligosaccharide) and/or an acceptor substrate (oligosaccharide or disaccharide) and an enzymatic transglycosidase reaction. The fermentation process produces the first substrate for the transglycosidase reaction, and the second substrate is supplied to the ongoing fermentation, where in the presence of a transglycosidase enzyme, the third (product) oligosaccharide is formed in the medium. The process further produces a side-product which is imported by the cells via a side-product importer (e.g., a lactose, lacto-N-biose or N-acetyllactosamine importer) in the fermentation to produce more of the first substrate.

One aspect is a method for producing a oligosaccharide from a donor oligosaccharide and an acceptor oligosaccharide or acceptor disaccharide, said method comprising the steps of:
a) cultivating a genetically modified cell capable of producing a first oligosaccharide of at least three monosaccharide units in a culture medium which is supplied with a carbon source, and wherein the genetically modified cell comprises one or more nucleic acids encoding:
   i) at least one side-product importer, and
   ii) at least one recombinant glycosyltransferase, and
   iii) at least one pathway to produce a nucleotide-activated sugar, and
b) supplying a second disaccharide or oligosaccharide to the culture medium, and
c) making an enzyme with transglycosidase activity available in the culture medium, and
d) incubating the first oligosaccharide, the second disaccharide or oligosaccharide and the transglycosidase enzyme in the culture medium in which the first oligosaccharide is produced to form a third oligosaccharide.

In embodiments the third oligosaccharide is a complex oligosaccharide of at least four monosaccharide units selected from the group consisting of DFL, FSL, Lewis B, Lewis Y, sialyl-Lewis A, sialyl-Lewis B, LNFP-I, LNFP-II, LNFP-III, LNFP-IV, LNFP-V, LNFP-VI, LST-a, LST-b, LST-c, DSLNT, LNDFH-I, LNDFH-II, LNDFH-III, FLST-a, FLST-b, FLST-c, pLNH, pLNnH, LNH, LNnH, FLNH-I, FLNH-II, FLNH-III, FpLNH-I, FpLNnH II, DF-LNF-I, DF-LNF-II, DF-LNF-III, DF-para-LNH, DF-para LNnH, FLNnHa, FLNnHb, DFLNnH, TF-LNH, SLNH, FSLNH, SLNnH-I, FSLNnH-I, SLNnH-II, and DS-FLNH-II.

In further embodiments the genetically modified cell produces a donor oligosaccharide selected from the group consisting of a fucosylated oligosaccharide of three to five monosaccharide units, a sialylated oligosaccharide of three to five monosaccharide units and a neutral core oligosaccharide three to four monosaccharide units. In preferred embodiments the donor oligosaccharide is selected from the group of HMOs consisting of 2′FL, 3FL, DFL, LNFP-I, FSL, 3′SL, 3′SLacNAc, 3′SLNB, 6′SL, LST-a, LNT-II, LNT and LNnT.

In other embodiments the genetically modified cell produces an acceptor oligosaccharide selected form the group consisting of 2′FL, 3FL, 2′FLacNAc, 2′FLNB, Lewis A, Lewis X, LNT-II, LNT, LNnT, para-LNnH, LNFP-I, LNFP-II, LNFP-III, LNFP-IV, LNFP-V, LNFP-VI, 3′SL, 6′SL, LST-a, and LST-c, which preferably are exported to the culture medium.

In further embodiments the second disaccharide or oligosaccharide is selected from the group consisting of LacNAc, LNB, 2′FL, 3FL, 2′FLacNAc, 2′FLNB, Lewis A, Lewis X, 3′SL, LNT, LNnT, LNFP-I, LNFP-II, LNFP-III, LNFP-V, LNFP-VI, LST-a, LST-c, LNH, F-LNH-II, F-LNH-III, DF-LNH-I, DF-LNH-II, DF-LNH-III, S-LNH, DS-LNH, FS-LNH, LNnH, Para-LNH and Para-LNnH. In embodiments where the second oligosaccharide is supplied to the culture medium by a second genetically engineered cell which is co-cultured with the first genetically engineered cell in the same culture medium the oligosaccharide produced by said second genetically engineered cell is preferably exported to the culture medium.

In further embodiments the transglycosidase enzyme is selected from the group consisting of α-1,2-tranfucosidase, alpha-1,3-transfucosidase, alpha-1,3/4-transfucosidase, alpha-2,3-transialylase, alpha-2,6-transsialylase, β-N-acetylglucosaminidase, trans-lacto-N-biosidase and trans-β-galactosidase. The transglycosidase enzyme can either be added to the culture medium during the fermentation or it can be expressed by the genetically engineered cell.

A further aspect described herein is the compositions produced by the hybrid method as disclosed in the examples. In particular, a composition of HMOs consisting essentially of:
a) at least 50 wt % FSL, between 20 to 45 wt % 3FL, and between 0.1 to 2 wt % 3′SL and between 0 to 3 wt % lactose, or
b) at least 50 wt % LST-a, between 15 to 40 wt % LNT, between 0 to 15 wt % 3′SL and between 0 to 2 wt % lactose, or
c) at least 50 wt % LST-c, between 15 to 25 wt % LNnT, between 15 to 25 wt % 6′SL and between 0 to 7 wt % lactose, and
where in the total composition constitute 100 wt % of the components and the composition is a mixture of at least two components.

A further aspect is the use of a composition of HMOs as described herein in the production of a nutritional composition.

### BRIEF DESCRIPTION OF FIGURES

FIG. 1: Non-limiting illustration of the hybrid fermentation-enzymatic process taking place in a fermentation bioreactor. An HMO producing strain in the reactor is fed with a carbon source (e.g. glucose glycerol, sucrose, etc) and an initial amount of lactose (lac) is provided to produce a first HMO. A sterilized 2^{nd} HMO is fed to the fermentation broth with the strain producing the first HMO. Once the 1^{st} and 2^{nd} HMOs are present in sufficient amounts a transglycosidase is added to the broth where it catalyzes the transfer a glycosyl moiety from the donor HMO (e.g., a sialyl- or fucosyl-lactose, which can be either the 1^{st} or 2^{nd} HMO) to the acceptor HMO thereby generating a 3^{rd} (glycosylated) HMO and lactose as side-product (the leaving group of the enzymatic step). The lactose is in turn taken up by the cell (recycled) producing the first HMO, thereby the equilibrium is pushed towards formation of the 3^{rd} complex HMO and feeding of lactose to the fermentation broth is not required resulting in a low level of lactose at the end of fermentation. The reverse enzymatic reaction is very low if the leaving group (lactose) is recycled faster than the enzymatic reaction rate indicated by the [symbol] .

FIG. 2: Shows the concentration profile curve in the hybrid process in weight percentage relative to total mole of substrates (mass fraction %) and products illustrating the process progress for the synthesis of FSL using the 3FL strain for in situ 3FL formation from lactose and by adding sterilized 3′SL solution into the medium. Trans-sialylation of 3FL (acceptor) using 3′SL as a sialyl donor was initiated by adding α-2,3-transsialidase enzyme at pH 6.7 and 25° C. Lactose concentration is shown as the dotted line with circles, 3FL concentration is the short hashed line with squares, 3′SL concentration is the long hashed line with triangles and FSL concentration is the full line with diamonds.

FIG. 3: Shows the concentration profile curve in a hybrid process in weight percentage relative to the total weight of substrates (mass fraction %) and products illustrating the process progress for the synthesis of LNDFH-I using a 3FL strain for in situ 3FL formation from lactose and by adding equimolar amount of LNFP-I to lactose into the medium. Trans-fucosylation of LNFP-I (acceptor) using 3FL as a fucosyl donor was initiated by adding sterile filtered α-1,3/4-transfucosidase (BiTF-641) at pH 6.8 and 25° C. Lactose concentration is shown as the dotted line with circles, 3FL concentration is the short dashed line with squares, LNFP-I concentration is the long dashed line with triangles and LNDFH-I concentration is the full line with diamonds.

FIG. 4: Shows the in vitro enzymatic reaction progress curve in weight percentage relative to the total weight of substrates (mass fraction %) of α-1,3/4-transfucosidase (BiTF-641) catalyzed trans-fucosylation of LNFP-I (acceptor) utilizing 3FL as a fucosyl donor for the synthesis of LNDFH-I starting at equimolar amount of LNFP-I to 3FL adding α-1,3/4-transfucosidase (BiTF-641) at pH 6.55 and 25° C. Lactose concentration is shown as the dotted line with circles, 3FL concentration is the short dashed line with squares, LNFP-I concentration is the long dashed line with triangles and LNDFH-I concentration is the full line with diamonds.

FIG. 5: Shows the concentration profile curve in a hybrid process in weight percentage relative to the total weight of substrates and products (mass fraction %) illustrating the process progress for the synthesis of LNDFH-I using a 3FL strain for in situ 3FL formation from lactose and by adding excess molar amount of LNFP-I to lactose (i.e. LNFP-I/lactose 2:1, mole/mole) into the medium. Trans-fucosylation of LNFP-I (acceptor) using 3FL as a fucosyl donor was initiated by adding α-1,3/4-transfucosidase (BiTF-641) at pH 6.8 and 25° C. Lactose concentration is shown as the dotted line with circles, 3FL concentration is the short dashed line with squares, LNFP-I concentration is the long dashed line with triangles and LNDFH-I concentration is the full line with diamonds.

FIG. 6: Shows the in vitro enzymatic reaction progress curve in weight percentage relative to the total weight of substrates (mass fraction %) of α-1,3/4-transfucosidase (BiTF-641) catalyzed trans-fucosylation of LNFP-I (acceptor) utilizing 3FL as a fucosyl donor for the synthesis of LNDFH-I starting at excess molar amount of LNFP-I to 3FL adding α-1,3/4-transfucosidase (BiTF-641) at pH 6.5 and 37° C. Lactose concentration is shown as the dotted line with circles, 3FL concentration is the short dashed line with squares, LNFP-I concentration is the long dashed line with triangles and LNDFH-I concentration is the full line with diamonds.

FIG. 7: Shows the concentration profile curve in a two-strain hybrid process in weight percentage relative to the total weight of substrates and products (mass fraction %) illustrating the process progress for the synthesis 6′SL (strain MF5) and a LNnT (strain MF6) and the decline of lactose as the two HMOs are produced. 6′SL acts as donor substrate and LNnT is acceptor in the transsialyation reaction that is initiated at 113 h after the start of the fermentation when the α-2,6-transsialidase PITS-197 is added to the fermentation, at this point LST-c starts forming and 6′SL and LNnT levels are reduced while lactose is being formed which stabilizes the 6′SL and LNnT production. Lactose concentration is shown as the dotted line with circles, 6′SL concentration is the short dashed line with squares, LNnT concentration is the long dashed line with triangles and LST-c concentration is the full line with diamonds.

FIG. 8: Shows the in vitro enzymatic reaction progress curve in weight percentage relative to the total weight of substrates (mass fraction %) of a α-2,6-transsialidase (PITS-197) catalyzed transsialyation of LNnT (acceptor) utilizing 6′SL as a sialyl donor for the synthesis of LST-c. Lactose concentration is shown as the dotted line with circles, 6′SL concentration is the short dashed line with squares, LNnT concentration is the long dashed line with triangles and LST-c concentration is the full line with diamonds.

FIG. 9: Non-limiting illustration of the two-strain hybrid fermentation-enzymatic process taking place in a fermentation bioreactor. A first oligosaccharide/HMO producing strain in the bioreactor is fed with a first carbon source (e.g. glucose, glycerol, sucrose, fructose, galactose, arabinose, sorbitol, maltose etc) and an initial amount of lactose (lac) is provided to produce a first oligosaccharide/HMO. The bioreactor further contains a second oligosaccharide/HMO producing strain which is fed with a second carbon source that is different from the first carbon source (e.g. glucose glycerol, sucrose, fructose, galactose, arabinose, sorbitol, maltose etc) and which produces a second oligosaccharide/HMO from the lactose. Preferably, both strains are inoculated into the bioreactor at the beginning of the fermentation, their growth and the production rate of their respective oligosaccharides can be controlled by feeding the two carbon sources at different rates. The first and second oligosaccharide/HMOs produced by the cells can serve as donor and acceptor in the transglycosylation reaction once they are present in sufficient amount in the medium. The transglycosylation is catalyzed by a transglycosidase which is provided to the medium, e.g., by addition or produced by one of strains. Once the transglycosidase is present in the medium of the running fermentation it catalyzes the transfer a glycosyl moiety from the donor oligosaccharide/HMO (e.g., a sialyl- or fucosyl-lactose), to the acceptor oligosaccharide/HMO thereby generating a third sialylated or fucosylated complex HMO and lactose as side-product (the leaving group of the enzymatic step). The lactose is in turn taken up by the first and the second strain (recycled) producing more of the first and the second oligosaccharides/HMOs, thereby the equilibrium is pushed towards formation of the third complex HMO. Towards the end of the fermentation feeding of lactose to the fermentation broth can be stopped and the residual lactose consumed until the end of fermentation. The reverse enzymatic reaction is very low if the leaving group (lactose) is recycled faster than the enzymatic reaction rate, which is indicated by the [symbol] .

FIG. 10: Shows the concentration profile curve in a one-strain hybrid process in weight percentage relative to the total weight of substrates and products (mass fraction %) illustrating the process progress adding 6′SL to an LNnT strain culture and the decline of lactose as the LNnT is produced. 6′SL acts as donor substrate and LNnT is acceptor in the transsialyation reaction initiated with the addition of α-2,6-transsialidase PITS-197 to the fermentation, at this point LST-c starts forming and 6′SL and LNnT levels are reduced. Lactose concentration is shown as the dotted line with circles, 6′SL concentration is the short dashed line with squares, LNnT concentration is the long dashed line with triangles and LST-c concentration is the full line with diamonds.

FIG. 11 illustrates the development of lactose, LNnT, 3FL and LNFP-III in weight % relative to the total weight of the substrates and products in a one-strain process. 3FL acts as donor substrate (produced from 3FL-S2 strain of table 12) and LNnT is acceptor in the transfucosylation reaction initiated with the addition of the α1,3/4-transfucosidase BiTF-641 to the fermentation, at this point LNFP-III starts forming and 3FL and LNnT levels are reduced. Lactose concentration is shown as the dotted line with circles, 3FL concentration is the short dashed line with squares, LNnT concentration is the long dashed line with triangles and LNFP-III concentration is the full line with diamonds.

FIG. 12: Shows the in vitro enzymatic reaction progress curve in weight percentage relative to the total weight of substrates (mass fraction %) of α-1,3/4-transfucosidase (BiTF-641) catalyzed trans-fucosylation of LNnT (acceptor) utilizing 3FL as a fucosyl donor for the synthesis of LNFP-III starting at a 3:1 molar ratio of LNnT to 3FL. Lactose concentration is shown as the dotted line with circles, 3FL concentration is the short dashed line with squares, LNnT concentration is the long dashed line with triangles and LNFP-III concentration is the full line with diamonds.

FIG. 13: illustrates the development of lactose, LNT, 3FL and LNFP-II in weight % relative to the total weight of the substrates and products in a one-strain process. 3FL acts as donor substrate (produced from 3FL-S1 strain of table 12) and LNT is acceptor in the transfucosylation reaction initiated with the addition of the α1,3/4-transfucosidase BiTF-641 to the fermentation, at this point LNFP-II starts forming and 3FL and LNT levels are reduced. Lactose concentration is shown as the dotted line with circles, 3FL concentration is the short dashed line with squares, LNnT concentration is the long dashed line with triangles and LNFP-II concentration is the full line with diamonds.

FIG. 14: Shows the in vitro enzymatic reaction progress curve in weight percentage relative to the total weight of substrates (mass fraction %) of α-1,3/4-transfucosidase (BiTF-641) catalyzed trans-fucosylation of LNT (acceptor) utilizing 3FL as a fucosyl donor for the synthesis of LNFP-II starting at a 2:1 molar ratio of LNT to 3FL. Lactose concentration is shown as the dotted line with circles, 3FL concentration is the short dashed line with squares, LNT concentration is the long dashed line with triangles and LNFP-III concentration is the full line with diamonds.

FIG. 15 shows the development of lactose, LNT, 3′SL and LST-a in weight % relative to the total weight of the substrates and products in a one-strain process. 3′SL acts as donor substrate and LNT is acceptor (produced from a LNT strain in table 12) in the trans-sialylation reaction initiated with the addition of the α-2,3-transsialidase TcTS to the fermentation, at this point LST-a starts forming and 3′SL and LNT levels are reduced. Lactose concentration is shown as the dotted line with circles, 3′SL concentration is the short dashed line with squares, LNT concentration is the long dashed line with triangles and LST-a concentration is the full line with diamonds.

FIG. 16: Shows the in vitro enzymatic reaction progress curve in weight percentage relative to the total weight of substrates (mass fraction %) of α-2,3-transsialidase TcTS catalyzed trans-sialylation of LNT (acceptor) utilizing 3′SL as a fucosyl donor for the synthesis of LST-a starting at a 1:1 molar ratio of LNT to 3′SL. Lactose concentration is shown as the dotted line with circles, 3′SL concentration is the short dashed line with squares, LNnT concentration is the long dashed line with triangles and LST-a concentration is the full line with diamonds.

FIG. 17 shows the development of lactose, LNT, 3′SL and LST-a in weight % relative to the total weight of the substrates and products in a two-strain process. 3′SL acts as donor substrate and LNT is acceptor in the trans-sialylation reaction initiated with the addition of the α-2,3-transsialidase TcTS to the fermentation, at this point LST-a starts forming and 3′SL and LNT levels are reduced. Lactose concentration is shown as the dotted line with circles, 3′SL concentration is the short dashed line with squares, LNT concentration is the long dashed line with triangles and LST-a concentration is the full line with diamonds.

FIG. 18: Shows the experimental setup of the regeneration and viability assessment of lyophilized probiotics under pH 3.0 acidic conditions.

FIG. 19: Shows the regeneration and viability of lyophilized Lactobacillus rhamnosus (DSM 32550), incubated for 3 h at pH 3.0 and plated in two dilutions 1:100 (E-2), 1:1000 (E-3) A) is the control without HMOs B) is Lactobacillus rhamnosus (DSM 32550) in combination with an HMO mixture containing 55% LST-a and 45% LNT (mix5); C) is Lactobacillus rhamnosus (DSM 32550) in combination with an HMO mixture containing 65% LST-a and 55% LNT and 10% 3′SL (mix6); D) is Lactobacillus rhamnosus (DSM 32550) in combination with an HMO mixture containing 45% 3FL and 55% FSL (mix7); and E) is Lactobacillus rhamnosus (DSM 32550) in combination with an HMO mixture containing 25% LNnT and 50% LST-c and 25% 6′SL (mix 8).

FIG. 20: shows the results from the plates in FIG. 19 expressed as mean values (n=2) with standard deviation (SD) of colony-forming units (CFU) per milliliter calculated from Lactobacillus rhamnosus (DSM 32550) colonies on agar plates. A) CFU of mix 5 and 6 (mixtures with LST-a and LNT) counted on agar plates of dilution step E-2. B) CFU of mix 7 and 8 counted on agar plates of dilution step E-4.

FIG. 21: Shows the regeneration and viability of lyophilized Bifidobacterium longum (DSM 32946), incubated for 30 min at pH 3.0 and plated undiluted A) is the control without HMOs B) is Bifidobacterium longum (DSM 32946) in combination with an HMO mixture containing 55% LST-a and 45% LNT (mix5); C) is Bifidobacterium longum (DSM 32946) in combination with an HMO mixture containing 65% LST-a and 55% LNT and 10% 3′SL (mix6); D) is Bifidobacterium longum (DSM 32946) in combination with an HMO mixture containing 45% 3FL and 55% FSL (mix7); and E) is Bifidobacterium longum (DSM 32946) in combination with an HMO mixture containing 25% LNnT and 50% LST-c and 25% 6′SL (mix 8).

### DETAILED DESCRIPTION

The inventors of the present invention have elegantly found a way to obtain the best properties from the in vivo bioproduction system and the in vitro enzymatic production system of oligosaccharides, such as HMOs, by combining these into a hybrid production system combining a fermentation step and an enzymatic step in the same vessel. The fermentation step and the enzymatic step are combined in a circular loop where the second oligosaccharide substrate is added to the loop converted to the third product oligosaccharide leaving the loop.

The realization that an enzymatic transglycosylation reaction, allowing the formation of a complex HMO, could effectively be conducted in a running fermentation process, as illustrated in Examples 1, 3, 5, 7, 8, 9, 11 and 12 was rather surprising. In the hybrid process the conditions are dictated by the fermentation conditions (e.g., temperature, pH, oxygen, carbon dioxide, stirring etc.) and the reaction environment is significantly more complex with multiple substrates and metabolites in the fermentation broth and potentially including proteases released from the cells, compared to the conventional in vitro enzymatic processes illustrated in Examples 2, 4, 6, 8, 9 and 11 where there are only two initial substrates (acceptor and donor) and the enzyme. To the best of our knowledge, this is the first time an enzymatic reaction has been used to synthesize a larger molecule from two smaller molecules in a running fermentation, especially while in situ recycling the side-product from the enzymatic process to produce the substrate, i.e., going beyond degradation of unwanted molecules.

### Oligosaccharides

In the present context, the term “oligosaccharide” means a sugar polymer containing at least three monosaccharide units, i.e., a tri-, tetra-, penta-, hexa- or higher oligosaccharide. The oligosaccharide can have a linear or branched structure containing monosaccharide units that are linked to each other by interglycosidic linkages. In preferred embodiments the oligosaccharide comprises a lactose, lacto-N-biose or N-acetyllactosamine residue/moiety at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. sialic acid).

In embodiments of the present invention an oligosaccharide to be produced by the method described herein may have a lactose (Galβ1-4Glc) moiety or a lacto-N-biose (LNB or Galβ1-3GlcNAc) moiety or N-acetyllactosamine (LacNAc or Galβ1-4GlcNAc) moiety at the reducing end.

In embodiments the oligosaccharide produced by the method described herein comprises at least 3 monosaccharide units and a lacto-N-biose (LNB or Galβ1-3GlcNAc) moiety or a N-acetyllactosamine (LacNAc or Galβ1-4GlcNAc) moiety at the reducing end, such as for example is Lewis A (LeA or Galβ1-3[fucα1-4]GlcNAc) or Lewis X (LeX or Galβ1-4[fucα1-3]GlcNAc), or Lewis Y (LeY or Fucα1-2Galβ1-4[Fucα1-3]GlcNAc) or Lewis-B (LeB or Fucα1-2Galβ1-3[Fucα1-4]GlcNAc) or 3′sialyl-LNB (Neu5Ac-α2-3Galβ1-3-GlcNAc) or 3′sialyl-LacNAc (Neu5Ac-α2-3Galβ1-4-GlcNAc) or 6′sialyl-LacNAc (Neu5Ac-α2-6Galβ1-4-GlcNAc) or 6′sialyl-LLNB (Neu5Ac-α2-6Galβ1-3-GlcNAc) or sialyl-Lewis A (SLeA or Neu5Ac-α2-3Galβ1-3[fucα1-4]GlcNAc) or sialyl-Lewis X (SLeX or Neu5Ac-α2-3Galβ1-4[fucα1-3]GlcNAc).

In the context of the present disclosure complex oligosaccharides fall into three categories i) oligosaccharides composed of at least four monosaccharide units of which at least two are selected from a fucosyl and/or a sialyl moiety, ii) oligosaccharides composed of at least five monosaccharide units, preferably with at least on sialyl or fucosyl monosaccharide, iii) oligosaccharides composed of at least 6 monosaccharide units, preferably neutral non-fucosylated oligosaccharides. In the context of the present disclosure, a sub-category of the complex oligosaccharides are the highly complex oligosaccharides where at least one monosaccharide unit in the oligosaccharide comprises at least three glycosidic linkages to additional monosaccharide units.

### HMOs

Preferred oligosaccharides of the disclosure are human milk oligosaccharides (HMOs).

The term “human milk oligosaccharide” or “HMO” in the present context means a carbohydrate found in human breast milk. The HMOs have a core structure comprising a lactose unit/moiety at the reducing end that can be elongated by one or more beta-N-acetyl-lactosaminyl and/or one or more beta-lacto-N-biosyl units, and this core structure can be substituted by an alpha-L-fucopyranosyl (fucosylated) and/or an alpha-N-acetyl-neuraminyl moiety (sialylated). HMO structures are for example disclosed in by Xi Chen in Chapter 4 of Advances in Carbohydrate Chemistry and Biochemistry 2015 vol 72.

In the context of the present disclosure, lactose is not regarded as an HMO species, but a substrate for the process. It is preferred to reduce lactose as much as possible at the end of the process.

HMOs are either neutral or acidic. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Non-fucosylated neutral HMOs are also termed neutral core HMOs.

Examples of such neutral non-fucosylated HMOs (neutral core HMOs) include lacto-N-triose II (LNT-II), lacto-N-tetraose (LNT or Galβ1-3GlcNAcβ1-3Galβ1-4Glc), lacto-N-neotetraose (LNnT or Galβ1-4GlcNAcβ1-3Galβ1-4Glc), lacto-N-neohexaose (LNnH or Galβ1-4GlcNAcβ1-3(Galβ1-4GlcNAcβ1-6)Galβ1-4Glc), para-lacto-N-neohexaose (pLNnH or Galβ1-4GlcNAcβ1-3Galβ1-4GlcNAcβ1-3Galβ1-4Glc), para-lacto-N-hexaose (pLNH or Galβ1-3GlcNAcβ1-3Galβ1-4GlcNAcβ1-3Galβ1-4Glc) and lacto-N-hexaose (LNH or Galβ1-3GlcNAcβ1-3(Galβ1-4GlcNAcβ1-6)Galβ1-4Glc).

Examples of neutral fucosylated HMOs include 2′-fucosyllactose (2′FL or Fucα1-2Galβ1-4Glc), 3-fucosyllactose (3FL or Galβ1-4(Fucα1-3)Glc), difucosyllactose (DFL or LDFT or Fucα1-2Galβ1-4(Fucα1-3)Glc), lacto-N-fucopentaose I (LNFP-I or Fucα1-2Galβ1-3GlcNAcβ1-3Galβ1-4Glc), lacto-N-fucopentaose II (LNFP-II or Galβ1-3[Fuc-α1-4]GlcNAcβ1-3Galβ1-4Glc), lacto-N-fucopentaose III (LNFP-III or Galβ(1-4[Fuc-α1-3]GlcNAcβ1-3Galβ1-4Glc), lacto-N-fucopentose IV (LNFP-IV or Fuc-α1-2Galβ1-4GlcNAcβ1-3Galβ1-4Glc), lacto-N-fucopentaose V (LNFP-V or Galβ1-3GlcNAcβ1-3Galβ1-4[Fuc-α1-3]Glc), lacto-N-fucopentaose VI (LNFP-VI or Galβ1-4GlcNAcβ1-3Galβ1-4[Fucα1-3]Glc), lacto-N-difucohexaose I (LNDFH-I or Fucα1-2Galβ1-3[Fucα1-4]GlcNAcβ1-3Galβ1-4Glc), lacto-N-difucohexaose II (LNDFH-II or Galβ1-3[Fuc-(α1-4)]GlcNAcβ1-3Galβ1-4[Fucα1-3]Glc), lacto-N-difucohexaose III (LNDFH-III or Galβ(1-4[Fuc-(α1-3)]GlcNAcβ1-3Galβ1-4[Fucα1-3]Glc), fucosyl-lacto-N-hexaose I (FLNH-I or Fuc-α1-2Galβ1-3GlcNAcβ1-3(Galβ1-4GlcNAcβ1-6)Galβ1-4Glc), fucosyl-lacto-N-hexaose II (FLNH-II or Galβ1-3(Fuc-α1-4)GlcNAcβ1-3(Galβ1-4GlcNAcβ1-6)Galβ1-4Glc), fucosyl-lacto-N-hexaose III (FLNH-III or Galβ1-3GlcNAcβ1-3 ((Gal(β1-4(Fucα1-3)GlcNAcβ1-6))Galβ1-4Glc), fucosyl-para-lacto-N-hexaose I (FpLNH-I or Galβ1-3GlcNAcβ1-3Galβ1-4[Fuc-(α1-3)]GlcNAcβ1-3Galβ1-4Glc), fucosyl-para-lacto-N-neohexaose II (FpLNnH-II or Galβ1-4GlcNAcβ1-3Gal(fucα1-3)β1-4GlcNAcβ1-3Galβ1-4Glc), Difucosyl-Lacto-N-hexaose I (DF-LNH-1 or DF-LNHa or fuc-α1-2Galβ1-3GlcNAcβ1-3 ((Gal(β1-4(Fuc1-3)GlcNAcβ1-6))Galβ1-4Glc), Difucosyl-Lacto-N-hexaose II (DF-LNH-II or DF-LNHb or Galβ1-3(fuc-α1-4)GlcNAcβ1-3 ((Gal(β1-4(Fuc1-3)GlcNAcβ1-6))Galβ1-4Glc), Difucosyl-Lacto-N-hexaose III (DF-LNH-III or DF-LNHc or fuc-α1-2Galβ1-3(fuc-α1-4)GlcNAcβ1-3 (Galβ1-4GlcNAcβ1-6)Galβ1-4Glc), Difucosyl-para-lacto-N-hexaose (DF-para-LNH or Galβ1-3(fuc-α1-4)GlcNAcβ1-3Galβ1-4(fuc-α1-3)GlcNAcβ1-3Galβ1-4Glc), Difucosyl-para-lacto-N-neohexaose (DF-para LNnH or Galβ1-4[Fucα1-3]GlcNAcβ1-3Galβ1-4[Fuc-α1-3]-GlcNAcβ1-3Galβ1-4Glc), fucosyl-lacto-N-neohexaose a (FLNnHa), fucosyl-lacto-N-neohexaose b (FLNnHb), difucosyl-lacto-N-neohexaose (DFLNnH) and trifucosyl-lacto-N-hexaose (TF-LNH or fuc-α1-2Galβ1-3(fuc-α1-4)GlcNAcβ1-3 ((Galβ1-4(Fuc1-3)GlcNAcβ1-6))Galβ1-4Glc).

Examples of acidic HMOs include 3′-sialyllactose (3′SL or Neu5Ac-α2-3Galβ1-4-Glc), 6′-sialyllactose (6′SL or Neu5Ac-α2-6Galβ1-4-Glc), 3-fucosyl-3′-sialyllactose (FSL or Neu5Ac-α2-3Galβ1-4(Fucα1-3)Glc), 3′-sialyllacto-N-tetraose a (LST a or Neu5Ac-α2-3Galβ1-3GlcNAcβ1-3Galβ1-4Glc), fucosyl-LST a (FLST a or Neu5Ac-α2-3Galβ1-3(Fucα1-4)GlcNAcβ1-3Galβ1-4Glc), 6′-sialyllacto-N-tetraose b (LST b or Galβ1-3(Neu5Ac-α2-6)GlcNAcβ1-3Galβ1-4Glc), fucosyl-LST b (FLST b or Fucα1-2Galβ1-3(Neu5Ac-α2-6)GlcNAcβ1-3Galβ1-4Glc), 6′-sialyllacto-N-neotetraose (LST c or Neu5Ac-α2-6Galβ1-4GlcNAcβ1-3Galβ1-4Glc), fucosyl-LST c (FLST c or Neu5Ac-α2-6Galβ1-4GlcNAcβ1-3Galβ1-4(Fucα1-3)Glc), 3′-sialyllacto-N-neotetraose (LST d), fucosyl-LST d (FLST d), disialyl-lacto-N-tetraose (DSLNT or Neu5Ac-α2-3Galβ1-3(Neu5Ac-α2-6)GlcNAcβ1-3Galβ1-4Glc), Sialyl-para-lacto-N-neohexaose (S-pLNnH or Neu5Ac-α2-3Galβ1-4GlcNAcβ1-3Galβ1-4GlcNAcβ1-3Galβ1-4Glc), sialyl-lacto-N-hexaose (SLNH or Neu5Ac-α2-6Galβ1-4GlcNAcβ1-6(Galβ1-3GlcNAcβ1-3)Galβ1-4Glc), fucosyl-sialyl-lacto-N-hexaose (FSLNH or Neu5Ac-α2-6Galβ1-4GlcNAcβ1-6(Fucα1-2Galβ1-3GlcNAcβ1-3)Galβ1-4Glc), sialyl-lacto-N-neohexaose I (SLNnH-I or Neu5Ac-α2-3Galβ1-4GlcNAcβ1-6(Galβ1-4GlcNAcβ1-3Galβ1-4Glc), fucosyl-sialyl-lacto-N-neohexaose I (FSLNnH-I or Neu5Ac-α2-6Galβ1-4GlcNAcβ1-3(Galβ1-4(Fucα1-3)GlcNAcβ1-6)Galβ1-4Glc), sialyl-lacto-N-neohexaose II (SLNnH-II or Neu5Ac-α2-6Galβ1-4GlcNAcβ1-3(Galβ1-4GlcNAcβ1-6)Galβ1-4Glc) and Disialyl-fucosyl-lacto-N-hexaose II (DS-FLNH-II or Neu5Ac-α2-3Galβ1-3(Neu5Ac-α2-6)GlcNAcβ1-3(Galβ1-4(Fucα1-3)GlcNAcβ1-6)Galβ1-4Glc).

In the context of the present disclosure complex HMOs fall into three categories i) HMOs composed of at least four monosaccharide units of which at least one is a fucosyl or a sialyl moiety, preferably, if the complex HMO consist of four monosaccharide units it contains at least two monosaccharide units selected from a fucosyl and/or a sialyl moiety, e.g., DFL and FSL, ii) HMOs composed of at least five monosaccharide units, preferably with at least on sialyl or fucosyl monosaccharide, non-limiting examples being LNFP-I, LNFP-II, LNFP-V, LST-a, LST-c as well as many of the highly complex HMOs mentioned below and iii) HMOs composed of at least 6 monosaccharide units, preferably neutral non-fucosylated oligosaccharides such as pLNH-I, pLNnH LNH and LNnH. In the context of the present disclosure, a sub-category of the complex HMOs are the highly complex HMOs where at least one monosaccharide unit in the oligosaccharide comprises at least three glycosidic linkages to additional monosaccharide units. Non-limiting examples of highly complex HMOs are LNH, LNnH, LNFP-II, LNFP-III, LST-b, DSLNT, LNDFH-I, LNDFH-II, FLST-a, FLST-b, FpLNnH, FpLNnH-II, F-LNH-I, F-LNH-II, DF-LNH-I, DF-LNH-II, DF-LNH-III, TF-LNH, DFpLNH, DFpLNnH, S-LNFP-I, S-LNH, S-LNnH-I, FS-LNH, FS-LNnH-I and DS-F-LNH-II.

Preferably, the complex HMOs of the present invention are not readily exported from the cytosol to the supernatant if produced by fermentation. Complex HMOs produced by the hybrid method described herein requires the action of at least two enzymes. The two enzymes can for example be at least one glycosyltransferase present in the cytosol of the genetically engineered cell used in the process and a transglycosidase present in the culture medium of the fermentation. For example, FSL produced using the hybrid system of the present invention requires the presence of an alpha-1,3-fucosyltransferase enzyme inside the genetically modified cell to form 3FL which is exported to the culture medium, and an alpha-2,3-transsialidase enzyme in the culture broth to from FSL from the 3FL produced by the cell and 3′SL added to the culture medium. Export from the genetically modified cell into the culture medium may require the presence of a recombinant transport in the genetically modified cell. For non-limiting examples of suitable transporters see for example WO2010/142305, WO2021/148615, WO2021/148614, WO2021/148611, WO 2021/148610, WO2021/148620 and WO2021/148618.

In one method described herein, the complex oligosaccharide, such as a human milk oligosaccharide (HMO), having at least four monosaccharide units is a fucosylated and/or sialylated HMO of four monosaccharide units, such as DFL or FSL. Preferably, the complex HMO of four monosaccharide units is FSL.

In one method described herein, the complex oligosaccharide, such as a human milk oligosaccharide (HMO), having at least four monosaccharide units, is an fucosylated and/or sialylated oligosaccharide with five monosaccharide units, such as an oligosaccharide selected from the group consisting of LNFP-I, LNFP-II, LNFP-III, LNFP-IV, LNFP-V, LNFP-VI, LST-a, LST-b, LST-c and LST-d. Specifically, the fucosylated and/or sialylated HMO with five monosaccharide units may be selected from an HMO the group consisting of LNFP-I, LNFP-II, LNFP-III, LNFP-V, LNFP-VI, LST-a, LST-b and LST-c.

In one method described herein, the complex oligosaccharide, such as a human milk oligosaccharide (HMO), having at least four monosaccharide units is a fucosylated and/or sialylated HMO with six monosaccharide units. Specifically, the fucosylated and/or sialylated HMO with six monosaccharide units may be selected from the group consisting of DSLNT, LNDFH-I, LNDFH-II, LNDFH-III, FLST-a, FLST-b and FLST-c.

In one method described herein the complex oligosaccharide, such as a human milk oligosaccharide (HMO), having at least four monosaccharide units is a fucosylated and/or sialylated of seven or eight monosaccharide units, such as an HMO selected from the group consisting FLNH-I, FLNH-II, FLNH-III, FpLNH-I, FpLNnH II, DF-LNF-I, DF-LNF-II, DF-LNF-III, DF-para-LNH, DF-para LNnH, FLNnHa, FLNnHb, DFLNnH, TF-LNH, SLNH, FSLNH, SLNnH-I, FSLNnH-I, SLNnH-II, and DS-FLNH-II. Production of these HMOs may require the presence of three or more glycosyltransferase and/or transglycosidase activities.

In one method described herein the complex oligosaccharide, such as a human milk oligosaccharide (HMO), having at least four monosaccharide units is a neutral core HMO of at least six monosaccharide units, such as an HMO selected from the group consisting of pLNH, pLNnH, LNH and LNnH.

### Hybrid Production Method

The hybrid production system described in the present disclosure comprises a fermentation step and an enzymatic step which can be conducted in the same vessel, where the vessel does not contain any means of separating the components in the vessel.

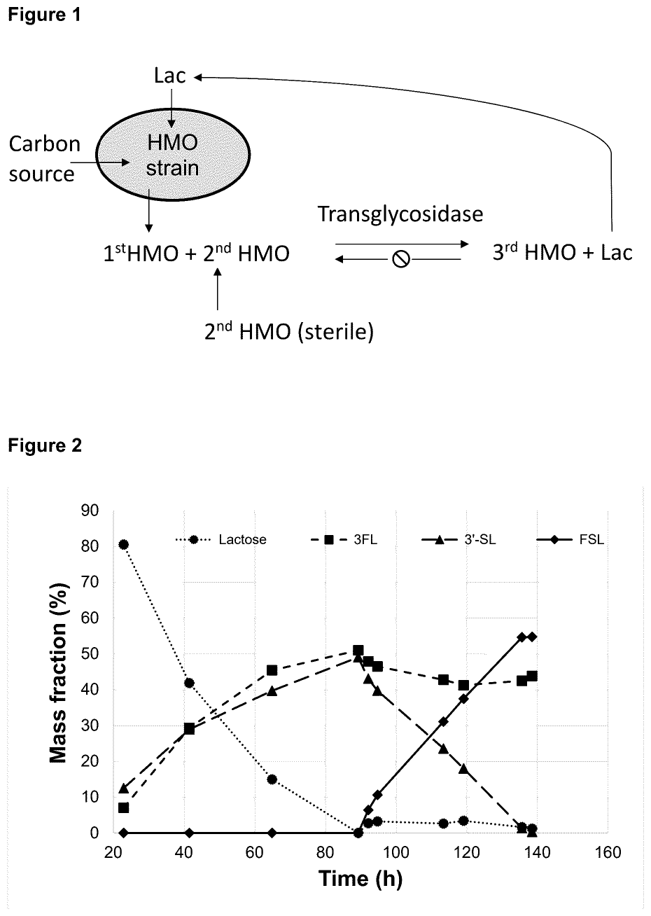

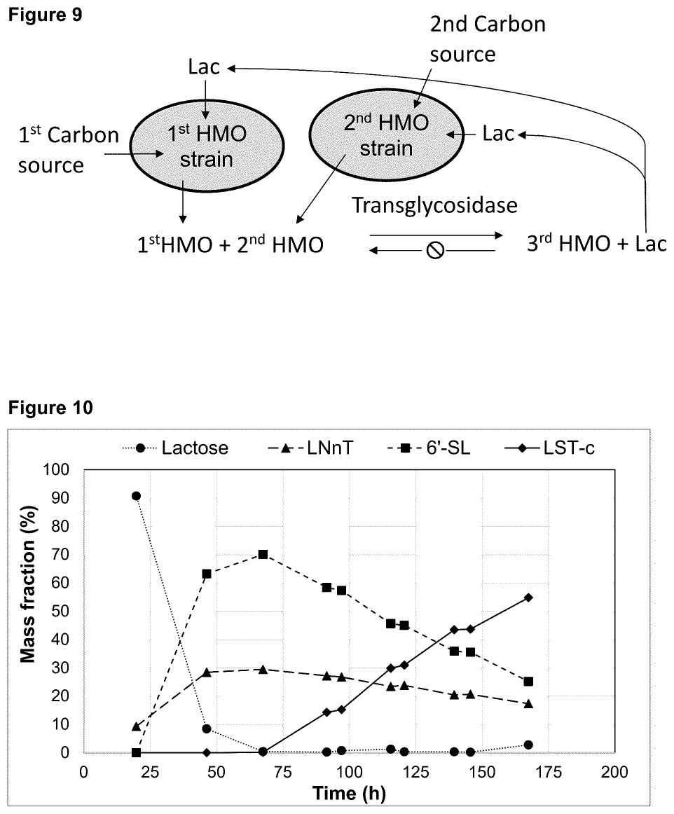

FIGS. 1 and 9 provide exemplary non-limiting illustrations of the hybrid process taking place in a fermentation bioreactor. In the hybrid process a genetically modified cell produces a first oligosaccharide which is secreted/exported into the culture medium of the fermentation. The first oligosaccharide can either act as a donor oligosaccharide or an acceptor oligosaccharide. If the third (product) oligosaccharide is a fucosylated and/or sialylated oligosaccharide and the first oligosaccharide is a donor oligosaccharide it contains a fucosyl- or sialyl-residue. The first oligosaccharide can also act as the acceptor oligosaccharide in the subsequent transglycosylation reaction occurring in the culture medium, in which case it serves as the backbone for the third (product) oligosaccharide. In embodiments where the hybrid process is a two-strain process the first and second oligosaccharides are independently produced by two genetically different strains in the same fermentation bioreactor (co-culturing). In this case one of the strains produce the donor oligosaccharide and the other strain produces the acceptor oligosaccharide.

In the context of the present invention, the term “donor oligosaccharide” is preferably understood as an oligosaccharide, which provides a specific moiety in a chemical reaction, e.g., a nucleophilic or electrophilic substitution reaction, to a further compound, preferably an acceptor. Likewise, the term “acceptor oligosaccharide” or “acceptor disaccharide” is preferably understood as an oligosaccharide or disaccharide, which receives a specific moiety in a chemical reaction, e.g., nucleophilic or electrophilic substitution reaction, from a donor, thereby forming a third compound.

Described herein is the production of an oligosaccharide from a donor oligosaccharide and an acceptor oligosaccharide or disaccharide, by cultivating a genetically modified cell capable of producing a first oligosaccharide of at least three monosaccharide units in a culture medium and supplying a second disaccharide or oligosaccharide to the culture medium and where an enzyme with transglycosidase activity is made available in the culture medium. By incubating the first oligosaccharide, the second disaccharide or oligosaccharide and the transglycosidase enzyme in the culture medium in which the first oligosaccharide is produced, a third oligosaccharide (product oligosaccharide) and a side-product (e.g., lactose, LNB or LAcNAc) is formed in the culture medium. In the case where the second disaccharide or oligosaccharide is a disaccharide, this preferably serves as acceptor in the transglycosidase process. In embodiments the acceptor is not lactose. In further embodiments the disaccharide acceptor may be lacto-N-biose (LNB) or N-acetyllactosamine (LacNAc), which are the backbones for Lewis A and Lewis X based structures.

In the production of oligosaccharides, such as complex HMOs, according to the method described here the first oligosaccharide is preferably an HMO. The genetically modified cell is engineered such that it effectively can produce the first oligosaccharide/HMO and preferably export it into the culture medium, where it is incubated with a second disaccharide/oligosaccharide/HMO, which is either added to the cultivation medium or produced by a second genetically modified strain, and a transglycosidase enzyme in the culture medium, to form a third complex oligosaccharide/HMO, such as a complex sialylated and/or fucosylated oligosaccharide/HMO of at least 4 monosaccharide units or a complex core oligosaccharide/HMO of at least 6 monosaccharide units.

An aspect described herein relates to a method for producing a complex oligosaccharide of at least four monosaccharide units from a donor oligosaccharide and an acceptor oligosaccharide, said method comprising the steps of:
a) cultivating a genetically modified cell capable of producing a first oligosaccharide of at least three monosaccharide units in a culture medium which is supplied with a carbon source, and wherein the genetically modified cell comprises one or more nucleic acids encoding:
   i) at least one side-product importer, such as a lactose importer, and
   ii) at least one recombinant glycosyltransferase, and
   iii) at least one pathway to produce a nucleotide-activated sugar, and
b) supplying a second oligosaccharide to the culture medium, and
c) making an enzyme with transglycosidase activity available in the culture medium, and
d) incubating the first oligosaccharide, the second oligosaccharide and the transglycosidase enzyme in the culture medium, in which the first oligosaccharide is produced, to form a complex oligosaccharide of at least four monosaccharide units.

A further embodiment described herein relates to a method for producing a sialylated and/or a fucosylated oligosaccharide of at least four monosaccharide units from a donor oligosaccharide and an acceptor oligosaccharide, said method comprising the steps of:
a) cultivating a genetically modified cell capable of producing a first oligosaccharide of at least three monosaccharide units in a culture medium which is supplied with a carbon source, and wherein the genetically modified cell comprises one or more nucleic acids encoding:
   i) at least one lactose importer, and
   ii) at least one recombinant glycosyltransferase, and
   iii) at least one pathway to produce a nucleotide-activated sugar, and
b) supplying a second oligosaccharide to the culture medium, wherein either the first or the second oligosaccharide is a fucosylated or sialylated donor oligosaccharide and the other oligosaccharide is an acceptor oligosaccharide, and
c) making an enzyme with transglycosidase activity available in the culture medium, wherein the enzyme with transglycosidase activity is
   i) a transfucosidase if the donor oligosaccharide is a fucosylated oligosaccharide, or
   ii) a transsialidase if the donor oligosaccharide is a sialylated oligosaccharide, and
d) incubating the first oligosaccharide, the second oligosaccharide and the transglycosidase enzyme in the culture medium in which the first oligosaccharide is produced to form a third sialylated and/or fucosylated oligosaccharide of at least four monosaccharide units.

In embodiments the donor oligosaccharide may be selected from the group consisting of a fucosylated oligosaccharide of three to five monosaccharide units, a sialylated oligosaccharide of three to five monosaccharide units and a neutral core oligosaccharide three to four monosaccharide units.

In the contexts of the hybrid production method the term “first oligosaccharide” or “first HMO” refers to the oligosaccharide produced in situ by the first genetically modified cell, and which constitute the first substrate in the enzymatic (transglycosidase) step of the hybrid process. The term “second disaccharide”, “second oligosaccharide” or “second HMO” refers to a disaccharide or an oligosaccharide that constitute the second substrate in the enzymatic (transglycosidase) step of the hybrid process. When the first oligosaccharide and the second disaccharide or oligosaccharide are reacted with the transglycosidase a third oligosaccharide (product oligosaccharide) and a side-product (leaving group) is produced. The leaving group is re-cycled by the genetically modified cell(s) to produce more of the first oligosaccharide and potentially second oligosaccharide, if this is supplied by a second genetically modified cell in the same culture as the first genetically modified cell. The third oligosaccharide is preferably the desired oligosaccharide, e.g., complex oligosaccharide, of the process, it may however also act as an intermediate oligosaccharide for a second transglycosidase reaction which produces a fourth oligosaccharide which is the desired complex oligosaccharide of the process. If the hybrid process comprises two enzymatic steps it can either be a two-step enzymatic process catalyzed by the same transglycosidase or by transglycosidases with different activity, e.g., one is a transfucosidase and the other is a transsialidase depending on their selectivity. However, in the case of using two different transglycosidases the other substrate for the second transglycosidase might be required to be supplied to the process.

The enzymatic transglycosidase reaction occurs in the culture medium and the first oligosaccharide produced by the genetically modified cell therefore needs to be available in the culture medium. Preferably, the first oligosaccharide is exported out of the cell without affecting the survival of the cell. In alternative embodiments, the first oligosaccharide may become available in the culture medium by natural lyses of a portion of the cells during the fermentation, without stopping the culture from growing.

In embodiments, it is desired that the genetically modified cell(s) exports the first and potentially second oligosaccharide produced by the cell(s) into the culture medium to make it easily available for the transglycosidase reaction in the culture medium. HMOs which can advantageously be produced and exported to the culture medium by the genetically modified cell(s) and which can serve as donor oligosaccharide in the transglycosylation reaction can be selected form the group consisting of 2′FL, 3FL, DFL, LNFP-I, 3′SL, 6′SL, 3′SLAcNAc, 3′SLNB, FSL, LST-a, LNT-II, LNT and LNnT.

In embodiments, HMOs which can advantageously be produced and exported to the culture medium by the genetically modified cell and serve as acceptor oligosaccharide in the transglycosylation reaction can be selected form the group consisting of 2′FL, 3FL, 2′FLacNAc, 2′FLNB, Lewis A, Lewis X, 3′SL, LNT, LNnT, LNFP-I, LNFP-II, LNFP-III, LNFP-V, LNFP-VI, LST-a and LST-c.

The term “supplying a second oligosaccharide to the culture medium”, is understood as making the second oligosaccharide available in the culture medium. This can for example be done by adding it from an external source or by supplying it in situ from a second genetically modified cell growing in the same culture as the first genetically modified cell. In an alternative embodiment the cell producing the first oligosaccharide may also produce the second oligosaccharide, allowing one cell to supply both the acceptor and donor oligosaccharide to the culture medium. Furthermore, such a cell may also produce the transglycosidase enzyme and supply this to the culture medium.

Essentially there is no limitation as to what second oligosaccharide can be added to the culture medium during the fermentation. Preferably, the second oligosaccharide is essentially free of other oligosaccharide compounds that can result in unwanted side-products. In the context of the present disclosure essentially free of other oligosaccharide compounds means that at least 90% of the total oligosaccharide is the 2^{nd} oligosaccharide, such as at least 95%, such as at least 98% of the total oligosaccharide is the 2^{nd} oligosaccharide. The addition of the second oligosaccharide to the fermentation is preferably done under sterile conditions. The second oligosaccharide can either act as a donor oligosaccharide, in which case the second oligosaccharide contains a fucosyl or sialyl residue, or it can act as the acceptor oligosaccharide in the subsequent transglycosylation reaction. In the production of complex HMOs, the second oligosaccharide is preferably an HMO.

In embodiments, oligosaccharides which can advantageously be supplied to the culture medium, and which can serve as donor oligosaccharide in the transglycosylation reaction can be selected form the group consisting of 2′FL, 3FL, DFL, LNFP-I, 3′SL, 6′SL, 3′SLAcNAc, 3′SLNB, FSL, LST-a, LNT-II, LNT and LNnT.

In embodiments, oligosaccharides which can advantageously be supplied to the culture medium and serve as acceptor oligosaccharide in the transglycosylation reaction can be selected form the group consisting of 2′FL, 3FL, 2′FLacNAc, 2′FLNB, Lewis A, Lewis X, 3′SL, LNT, LNnT, LNFP-I, LNFP-II, LNFP-III, LNFP-IV, LNFP-V, LNFP-VI, LST-a, LST-b, LST-c, LST-d, LNH, F-LNH-II, F-LNH-III, DF-LNH-I, DF-LNH-II, DF-LNH-III, S-LNH, DS-LNH, FS-LNH, LNnH, Para-LNH and Para-LNnH.

In alternative embodiments the second oligosaccharide is supplied to the culture medium from a second genetically modified cell capable of producing the second oligosaccharide. The second oligosaccharide can be in a more or less pure form and potentially just in the form of the filtered (cell free) fermentation broth obtained from cultivating the second genetically modified cell. In a preferred embodiments the second genetically modified cell capable of producing the second oligosaccharide is co-cultured with the first genetically modified cell, i.e., the cells grow in the same culture medium (See FIG. 9) and thereby supply the second oligosaccharide in situ. The advantage of generating both the first and the second oligosaccharide in situ. An advantage of the two-strain hybrid system is that it can save production capacity since both the donor and acceptor oligosaccharides can be produced in a single fermentation instead of having to produce the second oligosaccharides separately and then add it to the fermentation. The process will also be more economically since a separately produced and purified oligosaccharide will be more expensive than an oligosaccharide produced in situ.

The terms “culturing” or “fermenting” or “fermentation” are used interchangeably in the present description and refers to the growth to the genetically modified cell(s) (strain(s)) in a bioreactor with the purpose of producing the first oligosaccharide and potentially a second oligosaccharide. The strain(s) grows on a suitable carbon-source such as glucose, sucrose, galactose, arabinose, sorbitol, maltose, fructose, xylose and glycerol.

The genetically modified cell is capable of producing the first or second oligosaccharide from a substrate which is preferably added to the culture medium and taken up by the cell to serve as the initial substrate for the production of the first oligosaccharide, e.g., HMO. In cases where the third oligosaccharide has a N-acetyllactoseamine (LacNAc) lacto-N-biose (LNB) at the reducing end the initial substrate may be selected from N-acetyllactoseamine (LacNAc) lacto-N-biose (LNB), which is then decorated in the cell in a similar way as lactose to produce for example Lewis A, Lewis X, 3′SLacNAc, 3′SLNB, 2′FLacNAc or 2′FLNB. Most commonly lactose is used as the initial substrate, but LNT-II can potentially also serve as substrate for LNT or LNnT production or 2′FL or 3FL can serve as substrate for DFL production. In embodiments the substrate for the production of the first HMO can be selected from lactose, 2′FL, 3FL or LNT-II. Preferably, the initial substrate is selected from lactose or 2′FL. In a preferred embodiment the substrate for the production of the first HMO is lactose. As an alternative to adding the initial substrate for the production of the first oligosaccharide to the fermentation medium, the genetically modified cell may be further engineered to produce the initial substrate inside the cell (see for example WO2015/150328).

To achieve the in situ recycling of the side-product, it is advantageous if the genetically modified cell is capable of internalizing the initial substrate used in the production of the first oligosaccharide.

In embodiments the genetically modified cell is capable of internalizing lactose, N-acetyllactoseamine (LacNAc) lacto-N-biose (LNB), 2′FL, 3FL and/or LNT-II, depending on which compound is used as the initial substrate for making the first HMO and potentially second HMO. This initial substrate internalized by the cell(s) may correspond to the side-product produced by the transglycosidase reaction, which is thereby re-cycled. The internalization of the side-product is preferably facilitated by a side-product importer, such as a lactose permease. Preferably, at least one of the genetically modified cells uses lactose as the initial substrate, and if the side-product produced by the transglycosidase reaction is not lactose, lactose is fed to the culture during the fermentation. Also, in the embodiment where the fermentation is a co-culture and both cells use lactose as the initial substrate, lactose is preferably also feed to the culture during fermentation to avoid running out of the initial substrate.

In some embodiments, the genetically modified cell(s) is preferably capable of internalizing lactose or 2′FL added to the culture medium, which the cell then utilizes for the production of the first oligosaccharide (e.g., HMO). Some microbial cells have endogenous lactose uptake systems, for example in the form of a lactose permease, which is also capable of importing 2′FL. Lactose permeases can also be genetically engineered into the cell either as a heterologous protein or as an additional recombinant copy of the native gene, if a higher lactose uptake is desired.

A natural microbial fermentation follows four phases, namely the lag phase, the growth phase, the stationary phase and the death phase. In industrial fed-batch fermentations the cells grow in two phases, a first phase of rapid cell growth in a culture medium with either unrestricted access to a carbon source or restricted access following a rapidly increasing feeding profile which limits the carbon source, and a second phase of more controlled cell growth where the industrial product is often produced. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter. In the hybrid method described herein, the cultivation is preferably a fed-batch or a continuous fermentation. Preferably, the cultivation is started with an initial carbon source (batch phase), and when this is consumed the carbon source is fed at a desired rate to the culture medium throughout the fermentation (feeding phase). In some embodiments the feed rate is adjusted such that the culture grows under carbon limitation, where the carbon source is added continuously allowing formation of the product in this phase while the formation of additional biomass is limited. The industrial fermentation will always be stopped before significant cell death occurs.

In embodiments, the cultivation of the genetically modified cell is initiated in the presence of sufficient initial substrate for the cell to produce the desired oligosaccharide, such as the first oligosaccharide or second oligosaccharide. In some embodiments, sufficient initial substrate to produce the desired amount of the desired oligosaccharide is present at the initiation of the cultivation, such that no additional substrate is added to the culture medium after the initiation of the cultivation. In embodiments the initial substrate for producing the desired oligosaccharide is selected from lactose, LacNAc, LNB, 2′FL, 3FL and LNT-II. In preferred embodiments the substrate for producing the first oligosaccharide and potentially the second oligosaccharide is lactose or 2′FL, most preferred it is lactose.

In alternative embodiments, the initial substrate for producing the desired oligosaccharide, such as the first oligosaccharide or the second oligosaccharide, such as lactose, is added to the culture medium when the initial carbon source is consumed, thereby allowing initial growth of the cells before initiating the production of the first and/or the second oligosaccharide. The initial substrate, such as lactose, can either be added as a single portion or be feed separately or together with the carbon source and/or the second oligosaccharide.

In embodiments where the second oligosaccharide is added to the culture, it can be added at the beginning of the cultivation (batch phase) or at one or multiple timepoints during the cultivation or be fed continuously during the cultivation. Preferably, the addition of the second oligosaccharide is not continued until the end of the process to allow for its consumption before terminating the process. The amount of the second oligosaccharide is estimated based on the amount of the first oligosaccharide being produced such that the molar ratio of the first and second oligosaccharide are balanced to secure optimal formation of the third oligosaccharide. In one example the molar ratio of the first and second oligosaccharide is 1:1 (equimolar). In some embodiments, it may be advantages to have an excess of the second oligosaccharide over the first oligosaccharide, such as a 1.5:1-10:1 ratio of second oligosaccharide over first oligosaccharide, such as 1.5:1-5:1, such as 2:1, 3:1, 4:, 5:1, 6:1, 7:1, 8:1, 9:1, 10:1. For example in a two-strain hybrid approach to form a complex oligosaccharide, the affinity constants (Km's) of the enzyme for the donor and acceptor might not be the same. In order, to maximize the efficiency of the enzymatic reaction the donor and acceptor can therefore advantageously be supplied in a ratio that reflects the Km's. In the instances where one molecule of the first oligosaccharide is produced using one molecule of the initial substrate, such as lactose, the preferred ratio of the second oligosaccharide over the initial substrate, such as lactose, is the same as the ratio between second oligosaccharide and the first oligosaccharide. In preferred embodiments the ratio between second oligosaccharide and lactose is 1.5:1-10:1, such as 1.5:1-5:1, such as a 2:1, 3:1, 4:1, 5:1, 6:1, 7:1, 8:1, 9:1, 10:1 ratio between second oligosaccharide and lactose. To prevent contamination of the culture medium the second oligosaccharide is preferably sterilized prior to the addition to the cultivation medium.

In embodiments, the second oligosaccharide is continuously added to the culture medium during the fermentation at a rate balanced to the production of the first oligosaccharide produced by the genetically modified cell. The advantage of applying the second oligosaccharide continuously balanced to the formation of the first oligosaccharide is to secure the desired ratio of the first and second oligosaccharide up until the addition of the transglycosidase thereby securing optimal formation of the third oligosaccharide. In other embodiments, the second oligosaccharide is added to the culture medium at a faster rate than the rate of formation of the first oligosaccharide.

In order to be able to reduce the levels of side-product (e.g., lactose) produced as the leaving group in the transglycosylation process (enzymatic process) of the hybrid process, at the end of the process, it is desired that the feeding of the substrate for the cell to produce the first oligosaccharide (e.g., lactose) is stopped earlier than the end of the process. When lactose is formed together with the third fucosylated or sialylated oligosaccharide as a result the transglycosidase reaction the genetically modified cell will be able to internalize the lactose and thereby remove any lactose produced in the enzymatic process and convert it into additional first oligosaccharide. This, in addition to removing undesired lactose from the culture medium, also serves to push the equilibrium towards formation of additional third oligosaccharide/complex HMO, which therefore can be produced in higher ratios than either the first and/or second oligosaccharides or both as compared to the conventional enzymatic process.

In other embodiments, the side-product from the transglycosidase reaction may be an oligosaccharide, such as an oligosaccharide with three monosaccharide units, such as an HMO. In this case the genetically modified cell is preferably engineered such that it can take up the side-product oligosaccharide and use it as substrate for the production of the first oligosaccharide going into the transglycosylation process in the culture medium. For example, the genetically modified cell produces DFL which serves as fucosyl donor in the transfucosylation reaction which then results in 2′FL as side-product (leaving group). The 2′FL is then taken up by the DFL producing cell which is capable of using the 2′FL as substrate for the production of DFL. In this method, lactose will not be needed in the process and will therefore be very low at the end of the cultivation, and likewise the amount of 2′FL at the end of cultivation will be low, since it is constantly reused by the cell.

In embodiments, the weight % of the third oligosaccharide/complex HMO exceeds the weight % of the donor oligosaccharide at the end of the process. Preferably, the ratio between the third oligosaccharide and the first and/or second oligosaccharide is above 1.5:1, more preferably above 2:1 and more preferably 5:1. By above a certain ratio, is meant that the first number indicated in the ratio can be the number indicated or larger than the indicated number.

In the hybrid process described herein, the transglycosidase enzyme mediating the transglycosylation of the acceptor oligosaccharide with the fucosyl- or sialyl-moiety of the donor oligosaccharide is available in the culture medium of the fermentation.

In embodiments the transglycosidase enzyme is expressed from a recombinant nucleic acid in the genetically modified cell producing the first oligosaccharide and exported from the cell into the culture medium. The export of the transglycosidase enzyme can for example be facilitated using appropriate signal peptides.

For expression in E. coli the signal peptide can for example be selected from one of the following well known signal peptides

| Signal | | Amino Acid | SEQ ID |
|---|---|---|---|
| sequence | Origin | Sequence | NO: |
| pelB | Erwinia carotovora | MKYLLPTAAAG | 61 |
| | | LLLLAAQPAMA | |
| ompA | Escherichia coli | MKKTAIAIAVA | 62 |
| | | LAGFATVAQA | |
| dsbA | Escherichia coli | MKKIWLALAGL | 63 |
| | | VLAFSASA | |
| lamB | Escherichia coli | MMITLRKLPLA | 64 |
| | | VAVAAGVMSAQ | |
| | | AMA | |
| phoA | Escherichia coli | MKQSTIALALL | 65 |
| | | PLLFTPVTKA | |
| malE | Escherichia coli | MKIKTGARILA | 66 |
| | | LSALTTMMFSA | |
| | | SALA | |
| mglB | Escherichia coli | MNKKVLTLSAV | 67 |
| | | MASMLFGAAAH | |
| | | A | |
| dsbC | Escherichia coli | MKKGFMLFTLL | 68 |
| | | AAFSGFAQA | |

In other embodiments the transglycosidase enzyme is added exogenously to the culture medium during the cultivation of the genetically modified cell. When the enzyme is added exogenously it is preferably sterile filtered prior to the addition to avoid contamination of the culture. The transglycosidase is added to the hybrid process in sufficient activity to mediate the transglycosylation of the acceptor oligosaccharide with the donor oligosaccharide. If the activity of the enzyme is reduced during the fermentation, it may be advantages to add the enzyme when sufficient substrate has been produced by the genetically modified cell for it not to be rate limiting for the process. It may also be possible to add enzyme more than one time to the cultivation process.

In some embodiments the transglycosidase is added to the culture medium at a time point when the genetically modified cell has converted at least 50% of the initial lactose into the first oligosaccharide, such as at least 75% of the initial lactose, such as at least 85% of the initial lactose, such as at least 90% of the initial lactose, such as between 95% and 100% of the initial lactose. It is advantageous to allow formation of a sufficient amount of first oligosaccharide before initiating the transglycosylation reaction to make sure that the substrates for the enzymatic process do not become rate limiting and the unconverted lactose does not inhibit the reaction.

In embodiments no additional lactose is added to the culture medium after addition of the transglycosidase. The addition of lactose to the culture can be stopped at this point since the transglycosidase reaction will generate lactose that the cells can take up to continue the production of the first oligosaccharide.

At the end of the fermentation, it is desired to deactivate the transglycosidase enzyme to avoid the shift of equilibrium of the transglycosidase reaction once the formation of the first oligosaccharide stops due to the cessation of the carbon source feed which provides energy and carbon for the genetically modified cells that is required for the continued recycling of lactose. Preferably, the deactivation is done before the cells are harvested or immediately after the harvest. Non-limiting examples of deactivation the transglycosidase can be selected from i) heating the fermentation broth to a temperature that denatures the enzyme, ii) adding a protease to the culture broth at the end of fermentation to hydrolyze the enzyme or iii) change the pH of the culture such that it is outside the activity range of the enzyme or denatures the enzyme. If heating is used for deactivation, it is preferred that the broth is heated to at least 60° C., such as at least 70° C., such as at least 80° C., such as at least 90° C., such as at least 95° C. for at least 5 minutes, such as at least 10 minutes, such as at least 15 minutes. If a protease is used to deactivate the enzyme, it is preferably added in a sufficient activity to hydrolyze all the enzyme at least 10 min, such as at least 20 min such as at least 30 min prior to the harvest of the cells. If a change in pH is used to deactivate the enzyme, it is preferred to decrease the pH to below 5, preferably to between 3 to 5 such as between 3.5 to 4.5.

The terms “manufacturing” or “manufacturing scale” or “large-scale production” or “large-scale fermentation” or “industrial production”, are used interchangeably and in the meaning of the present disclosure defines a fermentation with a minimum volume of 100 L, such as 1000 L, such as 10,000 L, such as 100,000 L, such as 200,000 L culture broth. Usually, a “manufacturing scale” process is defined by being capable of processing large volumes, yielding amounts of the HMO product of interest that meet, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behavior of an expression system in a lab scale method, such as shake flasks, benchtop bioreactors or the deep well format described in the examples of the disclosure, does allow to predict the behavior of that system in the complex environment of a bioreactor.

With regards to the suitable culture medium used in the fermentation process, there are no limitations. The culture medium may be semi-defined, i.e., containing complex media compounds (e.g., yeast extract, soy peptone, casamino acids, etc.), or it may be chemically defined, without any complex compounds. The carbon-source can be selected from the group consisting of glucose, sucrose, fructose, xylose and glycerol. In one or more exemplary embodiments, the culturing media is supplemented with one or more carbon sources selected form the group containing glycerol, sucrose, fructose, galactose, maltose, arabinose, sorbitol, xylose and glucose. Alternative carbon sources can be selected from molasses, invert sugar, corn steep liquor, yeast extract, tryptone, acetate, corn syrup, succinate, malate, pyruvate, lactate, ethanol, methanol, xylanose, citrate and raffinose.

### Co-Culturing

The term “co-culture” or “co-culturing” a used in the present disclosure relates to growth of two different genetically modified cells (strains) in the same culturing vessel, such as a shake flask, a fermenter or a bioreactor, to produce their products in the same culture medium. Preferably, the two different strains are grown simultaneously in the culture medium. The two different strains may for example be inoculated into the vessel at the beginning of the cultivation. This allows the strains to grow simultaneously in the same vessel producing their products in the same culture medium. The second strain may however also be added to the vessel at a later timepoint if it is desired to give the first strain an opportunity to increase its biomass before the second strain is added. This will still result in simultaneous growth of the strains during some of the cultivation time. The two different strains may also be inoculated (pitched) in different ratios (cells/ml), if it is known that one strain has an initial slower growth than the other strain, if the oligosaccharide yield per mol of carbon source of the two strains are different, or if it is desired that the ratio of the products produced by the two strains is different. The term cell and strain are used interchangeably in the present disclosure.

Embodiments described herein relates to a method for producing a oligosaccharide, such as a complex oligosaccharide of at least four, such as at least five monosaccharide units, from a donor oligosaccharide and an acceptor oligosaccharide produced (supplied) by a first and a second genetically modified cell, said method comprising the steps of:
a) co-culturing a first and second genetically modified cell in a culture medium, wherein
   i) the first genetically modified cell is capable of producing a first oligosaccharide of at least three monosaccharide units and wherein the genetically modified cell
      comprises one or more recombinant nucleic acid sequences encoding at least one glycosyltransferase; and
      comprises at least one pathway to produce a nucleotide-activated sugar from the first carbon source; and
      is preferably capable of exporting said first oligosaccharide into the culture medium; and
   ii) the second genetically modified cell is capable of producing a second disaccharide or oligosaccharide, such as an oligosaccharide of at least three monosaccharide units, and wherein the genetically modified cell
      comprises one or more recombinant nucleic acid sequences encoding at least one glycosyltransferase, and
      comprises a biosynthetic pathway for making said activated sugar nucleotide from the second carbon source;
      is preferably capable of exporting said second oligosaccharide into the culture medium, and
b) making an enzyme with transglycosidase activity available in the culture medium, and
c) incubating the first oligosaccharide, the second oligosaccharide and the transglycosidase enzyme in the culture medium in which the first and second oligosaccharides are reacted to form a third complex oligosaccharide of at least four, such as five monosaccharide units.

Embodiments described herein relates to a method for producing a sialylated and/or a fucosylated oligosaccharide, such as a sialylated and/or a fucosylated oligosaccharide of at least four monosaccharide units, from a donor oligosaccharide and an acceptor disaccharide or acceptor oligosaccharide produced by a first and a second genetically modified cell, said method comprising the steps of:
a) co-culturing a first and second genetically modified cell in a culture medium, wherein
   i) the first genetically modified microbial cell is capable of producing a first oligosaccharide of at least three monosaccharide units and wherein the genetically modified cell
      comprises one or more recombinant nucleic acid sequences encoding at least one glycosyltransferase; and
      comprises at least one pathway to produce a nucleotide-activated sugar from the first carbon source; and
      is preferably capable of exporting said first oligosaccharide into the culture medium; and
   ii) the second genetically modified microbial cell is capable of producing a second oligosaccharide of at least three monosaccharide units and wherein the genetically modified cell
      comprises one or more recombinant nucleic acid sequences encoding at least one glycosyltransferase, and
   comprises a biosynthetic pathway for making said activated sugar nucleotide from the second carbon source;
   is preferably capable of exporting said second oligosaccharide into the culture medium; and
   wherein either the first or the second oligosaccharide is a fucosylated or sialylated donor oligosaccharide and the other disaccharide or oligosaccharide is an acceptor disaccharide or acceptor oligosaccharide, and
b) making an enzyme with transglycosidase activity available in the culture medium, wherein the enzyme with transglycosidase activity is
   i) a transfucosidase if the donor oligosaccharide is a fucosylated oligosaccharide, or
   ii) a transsialidase if the donor oligosaccharide is a sialylated oligosaccharide, and
c) incubating the first oligosaccharide, the second oligosaccharide and the transglycosidase enzyme in the culture medium in which the first and second oligosaccharides are produced to form a third sialylated and/or fucosylated oligosaccharide of at least four monosaccharide units.

In order to control the growth of two different genetically modified cells in the same culture it is advantageous if they grow on non-identical carbon sources as illustrated in FIG. 9. Ideally the strains are i) not capable of growing on or ii) have limited growth on the carbon source used by the other strain in the co-culture.

When operating with two different strains feeding on two different carbon sources the fermentation may essentially contain two batch phases running simultaneously, one for each strain/carbon source. Preferably, the amounts of the two carbon-sources are adjusted such that the duration of the two batch phases are similar. The feeding rates of the two carbon sources can be predicted based on knowledge of the individual growth rates and oligosaccharide product yields of the first and second strains. If a specific ratio of oligosaccharides is desired, this can be achieved by balancing the ratio of the two carbon sources in the feed based on the oligosaccharide/carbon source yield of the individual strains. As indicated above the batch phases may also be staggered, if it is desired to start the growth of one strain ahead of the other strain. The industrial fermentation will always be stopped before significant cell death occurs.

In order for the first and second genetically modified cells to grow on different carbon sources and not on the same carbon source it may be necessary to select cells with certain growth properties or genetically modify the cells such that they have the desired growth properties.

Microorganisms are often capable of using more than one carbon source to facilitate its growth. To establish whether a cell can grow on a certain carbon source one may for example spread the microorganism on an agar plate with the selected carbon source and observe the formation of colonies.

Depending on the species of the microbial strain used in the context of the present description different modifications may be needed to secure that a strain does not grow or has limited growth on the desired carbon source.

In the context of the co-culturing described herein, limited or reduced growth of a genetically modified cell refers to a cell that has a reduced affinity and uptake rate for a specific carbon source (low affinity strain) which means it cannot effectively compete with a strain having a higher affinity for the same specific carbon source. This is especially the case when the growth on the specific carbon source is under carbon limited conditions such as in the feeding phase of a carbon limited fed-batch or a continuous culture, in such a case the higher affinity strain will lower the residual concentration of the specific carbon source in the medium to such low levels that the low affinity strain has almost no growth on that carbon source. Moreover, even in a batch phase with excess carbon source the low affinity strain is at a major disadvantage compared to the high affinity strain as the maximum growth rate is also affected by lack of a main carbon source uptake system for the specific carbon source (e.g., deletion of the ptsG when the specific carbon source is glucose) since the maximum carbon source uptake rate, and thereby also maximum growth rate, is affected.

Gram negative cells are known to have a periplasmic space between the inner cytoplasmic membrane and the bacterial outer membrane. Gram positive bacteria can also have a periplasmic space although this is often significantly smaller. In terms of prevention or limiting growth of a specific carbon source in a microbial cell an option is to prevent the carbon source to enter the cytosol of the microbial cell, hence a carbon source may enter the periplasmic space, but if it is prevented to enter the cytosol the cell may still not be able to grow on it. A further option is to prevent the cell to further process the carbon source once it enters the cytosol, such that it cannot enter energy producing pathways such as glycolysis, pentose phosphate pathway or the Krebs cycle which the cell needs to grow. This can be achieved by denying the cell access to the enzyme needed to for example phosphorylate the carbon source. This entails abolishing its uptake via the PTS-uptake system if such as system is present for the carbon source and removing the enzyme responsible for its phosphorylation inside the cytosol.

In embodiments the ability to grow on a first carbon source and not on a second carbon source is achieved by securing that the cells express the right transporters for the selected carbon source while at the same time not having efficient transporters for the second carbon source. If the desired transporters are or are not naturally present in the host cell, the cell can be genetically engineered to exhibit the desired carbon source utilization.

In the sections below transport and utilization of the different carbon sources, glucose, glycerol, sucrose, galactose, arabinose, sorbitol, fructose and maltose is described. It is understood that in the method described herein the genetically modified cells may have functional transport/utilization of a sugar from one or more of these groups, or one or more of the transporter or utilization enzymes of a sugar from one or more of these groups may be reduced or abolished, e.g., by mutation or deletion of relevant genes described in the sections below.

In some embodiments the first genetically modified microbial cell grows on sucrose and has no or limited growth on glucose and the second genetically modified microbial cell grows on glucose and has no or limited growth on sucrose.

In some embodiments the first genetically modified microbial cell grows on sucrose and has no or limited growth on glycerol and the second genetically modified microbial cell grows glycerol and has no or limited growth on sucrose.

In some embodiments the first genetically modified microbial cell grows on sucrose and has no or limited growth on maltose and preferably also on glucose and the second genetically modified microbial cell grows maltose and has no or limited growth on sucrose.

In some embodiments the first genetically modified microbial cell grows on sucrose and has no or limited growth on galactose and the second genetically modified microbial cell grows galactose and has no or limited growth on sucrose.

In some embodiments the first genetically modified microbial cell grows on sucrose and has no or limited growth on fructose and the second genetically modified microbial cell grows fructose and has no or limited growth on sucrose.

In some embodiments the first genetically modified microbial cell grows on sucrose and has no or limited growth on arabinose and the second genetically modified microbial cell grows on arabinose and has no or limited growth on sucrose.

In some embodiments the first genetically modified microbial cell grows on sucrose and has no or limited growth on sorbitol and the second genetically modified microbial cell grows on sorbitol and has no or limited growth on sucrose.

In some embodiments the first genetically modified microbial cell grows on glucose and has no or limited growth on sucrose and the second genetically modified microbial cell grows on sucrose and has no or limited growth on glucose.

In some embodiments the first genetically modified microbial cell grows on glucose and has no or limited growth on glycerol and the second genetically modified microbial cell grows on glycerol and has no or limited growth on glucose.

In some embodiments the first genetically modified microbial cell grows on glucose and has no or limited growth on galactose and the second genetically modified microbial cell grows on galactose and has no or limited growth on glucose.

In some embodiments the first genetically modified microbial cell grows on glucose and has no or limited growth on fructose and the second genetically modified microbial cell grows on fructose and has no or limited growth on glucose.

In some embodiments the first genetically modified microbial cell grows on glucose and has no or limited growth on arabinose and the second genetically modified microbial cell grows on arabinose and has no or limited growth on glucose.

In some embodiments the first genetically modified microbial cell grows on glucose and has no or limited growth on sorbitol and the second genetically modified microbial cell grows on sorbitol and has no or limited growth on glucose.

In some embodiments the first genetically modified microbial cell grows on glycerol and has no or limited growth on sucrose and the second genetically modified microbial cell grows on sucrose and has no or limited growth on glycerol.

In some embodiments the first genetically modified microbial cell grows on glycerol and has no or limited growth on glucose and the second genetically modified microbial cell grows on glucose and has no or limited growth on glycerol.

In some embodiments the first genetically modified microbial cell grows on glycerol and has no or limited growth on galactose and the second genetically modified microbial cell grows on galactose and not or limited on glycerol.

In some embodiments the first genetically modified microbial cell grows on glycerol and has no or limited growth on fructose and the second genetically modified microbial cell grows on fructose and not or limited on glycerol.

In some embodiments the first genetically modified microbial cell grows on glycerol and has no or limited growth on maltose and preferably also on glucose and the second genetically modified microbial cell grows on maltose and not or limited on glycerol.

In some embodiments the first genetically modified microbial cell grows on glycerol and has no or limited growth on arabinose and the second genetically modified microbial cell grows on arabinose and has no or limited growth on glycerol.

In some embodiments the first genetically modified microbial cell grows on glycerol and has no or limited growth on sorbitol and the second genetically modified microbial cell grows on sorbitol and has no or limited growth on glycerol.

In some embodiments the first genetically modified microbial cell grows on galactose and has no or limited growth on maltose and preferably also on glucose and the second genetically modified microbial cell grows on maltose and not or limited on galactose.

In some embodiments the first genetically modified microbial cell grows on galactose and has no or limited growth on fructose and preferably also on glucose and the second genetically modified microbial cell grows on maltose and not or limited on galactose.

In some embodiments the first genetically modified microbial cell grows on fructose and has no or limited growth on maltose and preferably also on glucose and the second genetically modified microbial cell grows on maltose and not or limited on fructose.

In embodiments the one or more oligosaccharides produced by the co-culture is a mixture of at least two human milk oligosaccharides (HMOs). Preferably, the at least two oligosaccharides are harvested from the co-culture. The HMOs produced by the first and second genetically modified microbial cell may independently be selected from the group consisting of 2′FL, 3FL, 3′SL, 6′SL, DFL, LNT-II, LNT, LNnT, LNFP-I, LNFP-III, LNFP-V, LNFP-VI, FSL, LST-a, LST-b, LST-c, LST-d, LNDFH-II and LNDFH-III, DSLNT, pLNH, pLNnH, LNH, LNnH, (D)F-LNH-I, (D)F-LNH-II, (D)F-LNH-III, F-para-LNH-I, DF-para-LNH, DF-para-LNnH, TF-LNH, FLST b, FLST a, FLST-c, S-LNH, S-LNnH-I, FS-LNH, FS-LNnH-I, DS-F-LNH-II.

In preferred embodiments the HMOs produced by the first and second genetically modified microbial cell may independently be selected from the group consisting of 2′FL, 3FL, 3′SL, 6′SL, DFL, LNT-II, LNT, LNnT, LNFP-I, LNFP-III, LNFP-V, LNFP-VI, LST-a, LST-c, LNDFH-II and LNDFH-III.

### Glucose Transport and Utilization

Glucose is one of the most accepted carbon sources by microorganisms and there are multiple systems by which a microbial cell can take up glucose and convert it into energy for growth. Various glucose transport systems are well described, see for example Jaheris et al 2008 FEMS Microbiol Rev 32: 891-907 for bacteria, Fuentes et al. 2013 Microbial Cell Factories 12:42 for E. coli and Kim et al 2013 Biochimica et Biophysica Acta 1830: 5204-5210 for yeast.

In embodiments described herein, a cell that grow on glucose has at least one glucose transport system. The glucose transport system may be selected from the systems described in Jaheris et al, Fuents et al or Kim et al. Specifically the glucose import system may be selected from a phosphoenolpyruvate:sugar phosphotransferase systems (PTS) such as PTS-dependent glucose (glc) utilization system, PTS-dependent mannose (man) utilization system, PTS-dependent maltose (mal) utilization system, PTS-dependent beta-glucoside (bgl) utilization system or PTS-dependent N-acetylglucosamine (nag) utilization system. Alternative glucose transport systems are the galactose:H+ symporter GalP, glucose uptake protein GIcU, sodium/glucose transporter family (SGLT) or ABC transporters such as the galactose/glucose ABC transporter (mg/ABC) system, trehalose/maltose/sucrose/palatinose (TMSP)-ABC transporter (malEFG) system and glucose/mannose ABC transporter (glcEFG) system or MFS transporter systems such as glucose proton symporter (glcP) and glucose facilitator (glf) or hexose transporters (HXT).

In other embodiments reduction or prevention of import of glucose into the cytosol of a microorganism can be achieved by mutating or deleting one or more sequences encoding proteins that affect the glucose import capacity as described in the paragraph above.

To utilize the glucose to make energy the cell phosphorylates the glucose once it has entered the cytosol to glucose-6-phosphate which can enter the energy producing metabolic pathways such as glycolysis and pentose phosphate pathway. Blocking the formation of glucose-6-phospate may therefore also serve to prevent the cell to utilize glucose as a carbon source for growth.

In embodiments the genetically modified microbial cell is a bacterium that has reduced or no growth on glucose, wherein the functionality of one or more endogenous proteins involved in glucose import and utilization, in said cell can be reduced or abolished. Preferably the proteins are selected from the group consisting of
i) glucose PTS complex components IICB^{Glc} (ptsG, e.g., Uniprot accession nr P69786, or functional variants thereof);
ii) beta-glucoside PTS complex components IIABC^{Bgl} (bglF, e.g., Uniprot accession nr P08722, or functional variants thereof);
iii) mannose PTS complex components—IICD^{Man} (manX, e.g., Uniprot accession nr P69797, or functional variants thereof);
iv) N-acetylglucosamine PTS complex components IIABC^{Nag} (nagE, e.g., Uniprot accession nr P09323, or functional variants thereof);
v) maltose/maltodextrin transport system (malX, e.g., Uniprot accession nr P19642, or functional variants thereof);
vi) galactose/glucose high-affinity ABC transporter components (mglC, e.g., Uniprot accession nr P23200, or functional variants thereof);
vii) trehalose/maltose/sucrose/palatinose (TMSP)-ABC transporter (malF, e.g., Uniprot accession nr P02916 or functional variants thereof);
viii) trehalose/maltose/sucrose/palatinose (TMSP)-ABC transporter (malG, e.g., Uniprot accession nr P68183, or functional variants thereof);
ix) galactose permease (galP, e.g., Uniprot accession nr P0AEP1, or functional variants thereof);
x) glucose proton symporter (glcP, e.g., Uniprot accession nr O07563, or functional variants thereof);
xi) glucose facilitator (glf, e.g., Uniprot accession nr P37747 or P21906, or functional variants thereof);
xii) glucose uptake protein (glcU, e.g., Uniprot accession nr P40420, or functional variants thereof);
xiii) sodium/glucose transporter family (sg/T, e.g., Uniprot accession nr P96169, or functional variants thereof); and
xiv) glucokinase (glk, e.g., Uniprot accession nr P0A6V8, or functional variants thereof); and
xv) hexose transporters (HXT).

Where the proteins in i)-vi) and xv) are all part of various glucose import complexes which generally are composed of multiple proteins. The proteins in item vii)-xiii) are single protein transports identified in different bacterial species. The protein in xiv) is an example of a glucose utilization enzyme, which phosphorylates glucose once it has entered the cell. In context of the present disclosure, it is preferred to reduce or abolish the activity of the membrane bound transporter protein. Preferably, the gene to be mutated/deleted in the mentioned complex is indicated in brackets in italics.

In embodiments the genetically modified microbial cell is E. coli that has reduced (limited) or no growth on glucose, wherein the functionality of one or more endogenous proteins involved in glucose import and utilization, in said cell can be reduced or abolished. Preferably the proteins are selected from the group consisting of
i) glucose PTS complex components IICB^{Glc} (ptsG)
ii) beta-glucoside PTS complex components IIABC^{Blg} (bglF)
iii) mannose PTS complex components—IICD^{Man}, (manX)
iv) N-acetylglucosamine PTS complex components IIABC^{Nag} (nagE)
v) maltose/maltodextrin transport system (malX)
vi) galactose/glucose high-affinity ABC transporter components (mglC);
vii) galactose permease (galP); and/or
viii) glucokinase (glk).

In embodiments described herein at least one of the genetically modified cells has reduced or abolished activity of at least one PTS-dependent sugar transport system selected from the group consisting of:
i) glucose PTS complex components IICB^{Glc};
ii) beta-glucoside PTS complex components—IIABC^{Bgl};
iii) mannose PTS complex components—IICD^{Man};
iv) N-acetylglucosamine PTS complex components—IIABC^{Nag}; and
v) Maltose/maltodextrin PTS complex—IICB^{malX}
vi) sorbitol PTS complex IICB^{slr}.

Preferably, at least the ptsG gene of the glucose PTS complex components IICB^{Glc} is deleted in a bacteria, such as E. coli, that has no or limited growth on glucose.

### Glycerol Transport and Utilization

In embodiments described herein, a cell that grow on glycerol has at least one glycerol transport system. The glycerol transport system may be selected from the glycerol facilitator (glpF) or glycerol/H⁺-symporter (stl1).

In other embodiments reduction or prevention of import of glycerol into the cytosol of a microorganism can be achieved by mutating or deleting one or more sequences encoding proteins that affect the glycerol import capacity, such as deletion or mutation of nucleic acid sequences encoding glycerol facilitator (glpF) or glycerol/H⁺-symporter (stl1).

To utilize the glycerol to make energy the cell phosphorylates the glycerol once it has entered the cytosol to glycerol-3-phosphate, the phosphorylation is conducted by the glycerol kinase (glpK). Blocking the formation of glycerol-3-phospate may therefore also serve to prevent the cell to utilize glycerol as a carbon source for growth. More information on glycerol utilization in various bacteria can be found in the review by Lin Ann. Rev. Microbial. 1976 30:535-78.

In embodiments the genetically modified cell has a reduced or no growth on glycerol wherein the functionality of one or more endogenous proteins involved in glycerol import and utilization, in said cell can be reduced or abolished. Preferably the reduction or abolished activity of the proteins is achieved by deletion or mutation of one or more nucleic acid sequences encoding a protein selected from the group consisting of glpF (e.g., Uniprot accession nr, or functional variants thereof), stl1 (e.g., Uniprot accession nr, or functional variants thereof) and glpK (e.g., Uniprot accession nr, or functional variants thereof).

In the event that it is desired that a bacteria, such as E. coli, has reduced or no growth on glycerol the functionality of the glycerol transporter protein, also known as the glycerol facilitator (glpF) is preferably reduced or abolished, e.g. by mutating or deleting the glpF gene in said cell. In addition, the activity of the glycerol kinase may be reduced or abolished, e.g., by mutating or deleting the glpK gene in said cell.

In embodiments of the method described herein one of the genetically modified cell has are reduced or abolished functionality of the proteins involved in glucose and/or glycerol import and utilization by full or partial inactivating one or more of the gene genes selected from the group consisting of ptsG, bglF, manX, nagE, malX, mglC, glk and glpF.

### Sucrose Transport and Utilization

In embodiments described herein, a cell that grows on sucrose has at least one sucrose transport system. The sucrose transport system may be the PTS-dependent sucrose (suc) utilization system. Alternatively, cells can grow on sucrose by having active sucrose invertase or sucrose hydrolase proteins in the outer membrane or the periplasmic membrane (if present), which are capable of cleaving sucrose to glucose and fructose which can then be taken up by the cell via relevant fructose and glucose transport systems (see for example WO 2013/087884).

In embodiments the genetically modified microbial cell capable of growing on sucrose comprises one or more nucleic acid sequences encoding a PTS-dependent sucrose utilization system. The PTS-dependent sucrose utilization system can for example be encoded by scrY, scrA, scrB and optionally scrR (see for example WO2015/197082), where the gene scrA codes for the sucrose transport protein Enzyme IIScr (e.g., SEQ ID NO: 48 or ncbi sequence ID: CAA40658.1 or functional variants thereof) that provides intracellular sucrose-6-phosphate from extracellular sucrose via an active transport through the cell membrane and the concomitant phosphorylation. The sucrose specific ScrY porin (e.g., SEQ ID NO: 47 or ncbi sequence ID: CAA40657.1 or functional variants thereof encoded by scrY) facilitate the sucrose diffusion through the outer membrane. The ScrB invertase enzyme (e.g., SEQ ID NO: 49 or ncbi sequence ID: WP_000056853.1 or functional variants thereof encoded by scrB) splits the accumulated sucrose-6-phosphate by hydrolysis to glucose-6-phosphate and fructose. The scrR encodes the Lad family DNA-binding transcriptional regulator (e.g., SEQ ID NO: 50 or ncbi sequence ID: WP_000851062.1 or functional variants thereof).

The E. coli csc PTS dependent sucrose system is described in WO2015/150328 expressed from the cscABKR gene cluster (SEQ ID NO: 52) encoding, sucrose permase (e.g. cscB with UniProt accession nr P30000.1 or functional variants thereof), fructokinase (e.g., csck with GenBank accession nr EDV65567.1 or functional variants thereof), sucrose hydrolase (e.g. cscA with NCBI accession nr WP_175214520.1 or functional variants thereof), and a transcriptional repressor (e.g. cscR with GenBank accession nr. AJA27326.1 or functional variants thereof).

Alternatively, the genetically modified microbial cell capable of growing on sucrose comprises a nucleic acid encoding a sucrose invertase or sucrose hydrolase enabling the assimilation of sucrose by said cell. The sucrose invertase may for example be a glycoside hydrolase and a sucrose-6-phosphate hydrolase (e.g., SacC_Agal with the GeneBank ID: WP_103853210.1 a functional variant thereof) or a beta-fructofuranosidase (e.g., Bff with GeneBank ID: BAD18121.1, or a functional variant thereof). Since the sucrose hydrolase or sucrose invertase will convert the sucrose to glucose and fructose, e.g. in the periplasmic space of the cell, the cell should preferably be able to grow on either glucose or fructose, meaning that in a two-strain system the other genetically modified cell should have no or limited growth on fructose and/or glucose.

In embodiments where the carbon source is sucrose, and its assimilation is enabled by a sucrose invertase or sucrose hydrolase the other cell in a two-strain system should preferably grow on glycerol or galactose.

In embodiments the genetically modified cell has a reduced or no growth on sucrose wherein the functionality of one or more endogenous proteins involved in sucrose import and utilization, in said cell can be reduced or abolished. Preferably the reduction or abolished activity of the proteins is achieved by deletion or mutation of one or more nucleic acid sequences selected from the group encoding proteins in the PTS-dependent sucrose utilization system, e.g. by mutating or deleting the cscABKR-gene cluster (SEQ ID NO: 52, or functional variants thereof), if present and functional in the cell. In particular deletion or mutation of the sucrose permease gene, such as cscB (e.g., Uniprot accession nr P30000.1, or functional variants thereof) or scrY (e.g., Uniprot accession nr B1LQA1, or functional variants thereof) is relevant if a strain is to show no or reduced grow on sucrose. Many non-pathogenic E. coli cells have lost the ability to grow on sucrose and it is therefore often not necessary to mutate the cell to prevent it from growing on sucrose since it no longer has the ability to do so.

### Galactose Transport and Utilization

In embodiments described herein, a cell that grow on galactose has at least one galactose transport system. The galactose transport system may be selected from the galactose:H+ symporter GalP, the galactose/glucose ABC transporter (mglABC) system, the PTSLac (lacFE) system and/or the sodium/glucose transporter family (sglT).

Galactose imported into the cell via GalP, mglABC or sglT is converted into galactose-1-phosphate (Gal1P) via galactose kinase which is in turn metabolized via the Leloir pathway (galMKTE) to alpha-glucose-1-phosphat (G1P).

Galactose imported into the cell via PTSLac (lacFE) system is converted into Galactose-6-phosphate (Gal6P) and further metabolized to triose phosphates by the Tag6P pathway (lacABCD).

A cell growing on galactose preferably also have a functional galactose kinase (galK), functional Leloir pathway and/or Tag6P pathway.

In other embodiments reduction or prevention of import of galactose into the cytosol of a microorganism can be achieved by mutating or deleting one or more sequences encoding proteins that affect the galactose import capacity, such as deletion or mutation of nucleic acid sequences galactose:H+ symporter GalP, the galactose/glucose ABC transporter (mg/ABC) system, sodium/glucose transporter family (sglT) or the PTSLac (lacFE) system.

To utilize the galactose to make energy the cell phosphorylates the galactose to gal1P or gal6P. Mutating, deleting of blocking the enzymes converting gal to gal1P or gal6P may therefore also serve to prevent the cell to utilize galactose as a carbon source for growth.

In embodiments the genetically modified cell has a reduced or no growth on galactose wherein the functionality of one or more endogenous proteins involved in galactose import and utilization, in said cell can be reduced or abolished. Preferably the reduction or abolished activity of the proteins is achieved by deletion or mutation of one or more nucleic acid sequences selected from the group encoding galP (e.g., Uniprot accession nr P0AEP1, or functional variants thereof), mglC (e.g., Uniprot accession nr P23200, or functional variants thereof), lacF (e.g., Uniprot accession nr P24400 or functional variants thereof), galK (e.g., Uniprot accession nr P0A6T3, or functional variants thereof) and/or sglT (e.g., Uniprot accession nr P96169, or functional variants thereof).

### Fructose Transport and Utilization

In embodiments described herein, a cell that grow on fructose has at least one fructose transport system. The fructose transport system may be selected from the fructose PTS complex components IIABC^{Fru}, glucose PTS complex components IICB^{Glc}, the fructose transporter FruP.

Fructose imported into the cell via is converted into fructose-1-phosphate (fru1P) or fructose-6-phosphate (fru6P) via fructose kinases.

In other embodiments reduction or prevention of import of fructose into the cytosol of a microorganism can be achieved by mutating or deleting one or more sequences encoding proteins that affect the fructose import capacity, such as deletion or mutation of nucleic acid sequences encoding components of the fructose PTS complex components IIABC^{Fru}, glucose PTS complex components IICB^{Glc}, the fructose transporter FruP.

To utilize the fructose to make energy the cell phosphorylates the fructose to fru1P or fru6P. Mutating, deleting of blocking the enzymes converting fructose to fru1P or fru6P may therefore also serve to prevent the cell to utilize fructose as a carbon source for growth.

In embodiments the genetically modified cell has a reduced or no growth on fructose wherein the functionality of one or more endogenous proteins involved in fructose import and utilization, in said cell can be reduced or abolished. Preferably the reduction or abolished activity of the proteins is achieved by deletion or mutation of one or more nucleic acid sequences selected from the group encoding fruA (e.g., Uniprot accession nr P20966, or functional variants thereof), ptsG (e.g., Uniprot accession nr P69786, or functional variants thereof), or FruP (e.g., Uniprot accession nr F4TKS5 or functional variants thereof).

### Maltose Transport and Utilization

In embodiments described herein, a cell that grow on maltose has at least one maltose transport system. On such maltose transport system is the MalFGK ABC superfamily transport system which transports maltose across the cytoplasmic membrane of Escherichia coli. The MalFGK transport system is a heterotetrameric complex comprised of integral membrane proteins MalF and MalG, which associate with two units of the peripheral membrane protein MalK which possess ATP binding properties and hence may provide energy to the maltose permease encoded by malF and malG.

Alternatively, the maltose transport system may be selected from the maltose/maltodextrin PTS complex—IICB^{mal} (e.g., Uniprot accession nr P19642, or functional variants thereof), encoded by malX. The PTS enzyme-II protein encoded by malX is capable of recognizing both glucose and maltose as substrates.

In embodiments where the maltose/maltodextrin PTS complex is used for growth on maltose of one strain it is referred that the second strain has no or limited growth on glucose as well as on maltose.

Maltose imported into the cell is converted into glucose via amylomaltase (e.g., UniProt accession nr. P15977.2 or functional variants thereof) encoded by malQ or an alternative maltase from another species. The glucose is in turn phosphorylated as described in the “glucose transport” section above.

In other embodiments reduction or prevention of import of maltose into the cytosol of a microorganism can be achieved by mutating or deleting one or more sequences encoding proteins that affect the maltose import capacity, such as deletion or mutation of nucleic acid sequences encoding components of the MalFGK ABC superfamily transport system or the maltose PTS complex components IICB^{mal}. Furthermore, deletion of glucokinase (glk, e.g., Uniprot accession nr P0A6V8, or functional variants thereof) will prevent utilization of maltose as carbon source since the glucose needs to be phosphorylated to be converted into energy by the cell.

In embodiments the genetically modified cell has a reduced or no growth on maltose wherein the functionality of one or more endogenous proteins involved in maltose import and utilization, in said cell can be reduced or abolished. Preferably the reduction or abolished activity of the proteins is achieved by deletion or mutation of one or more nucleic acid sequences selected from the group encoding MalF (e.g., UniProt accession nr P02916.1 or functional variants thereof), MalG (e.g., GenBank: AAC77002.1 or functional variants thereof), MalK (e.g., UniProt accession nr. P02916.1 or functional variants thereof), PTS complex—IICB^{mal} (e.g., Uniprot accession nr P19642, or functional variants thereof) encoded by malX and/or glucose kinase (e.g., Uniprot accession nr P0A6V8, or functional variants thereof) encoded by glk.

### Arabinose Transport and Utilization

In embodiments described herein, a cell that grow on arabinose has at least one arabinose transport system.

On such arabinose transport system is the AraFGH arabinose transporter which is a member of the ATP Binding Cassette (ABC) transporter superfamily. The AraF is the periplasmic binding protein (e.g, UniProt accession nr. P02924 or functional variants thereof). AraH is the membrane component (e.g, UniProt accession nr. P0AE26 or functional variants thereof) and AraG is the ATP-binding component of this ABC transporter (e.g., UniProt accession nr. P0AAF3 or functional variants thereof).

Alternatively, the arabinose transport system may be selected from the arabinose-proton symporter AraE (e.g. UniProt accession nr. P0AE24 or P96710 or functional variants thereof).

In embodiments the genetically modified cell has a reduced or no growth on arabinose wherein the functionality of one or more endogenous proteins involved in aribinose import and utilization, in said cell can be reduced or abolished. For example, by prevention of import of arabinose into the cytosol of a microorganism by mutating or deleting one or more sequences encoding proteins that affect the arabinose import capacity, such as deletion or mutation of nucleic acid sequences encoding components of the AraFGH ABC superfamily transport system or the maltose arabinose-proton symporter AraE.

### Sorbitol Transport and Utilization

In embodiments described herein, a cell that grow on sorbitol has at least one sorbitol transport system.

The transport of sorbitol into procaryotic cells is facilitated by a phosphoenolpyruvate-dependent phosphotransferase system (PTS). The sorbitol-specific Enzyme IIB and IIC (EIIBC^{srl}) components, are responsible for binding to sorbitol and initiating its transport into the cell, this enzyme is encoded by srlA (e.g., Uniprot accession nr P56579 or O32333 or functional variants of these) and srl E, (e.g., Uniprot accession nr P56580 or O32332 or functional variants of these) respectively. As part of the PTS process, the incoming sorbitol molecule is simultaneously phosphorylated by sorbitol kinase (EIIA^{srl}) encoded by the gene srlB (e.g., Uniprot accession nr P05706 or A5I7D9 or functional variants of these).

In embodiments where the sorbitol PTS complex is used for growth on sorbitol of one strain it is referred that the second strain has no or limited growth on glucose as well as on maltose.

In embodiments the genetically modified cell has a reduced or no growth on sorbitol wherein the functionality of one or more endogenous proteins involved in sorbitol import and utilization, in said cell can be reduced or abolished. For example, by prevention of import of sorbitol into the cytosol of a microorganism by mutating or deleting one or more sequences encoding proteins that affect the sorbitol PTS system.

Irrespective of the which of the above carbon source transport systems is chosen, it is desired that the first genetically modified cell exports the first oligosaccharide produced by the cell into the culture medium and second genetically modified cell exports the second oligosaccharide produced by the cell into the culture medium to make them easily available for the transglycosidase reaction in the culture medium.

In embodiments, HMOs which can advantageously be produced and exported to the culture medium by one of the genetically modified cells and which can serve as donor oligosaccharide in the transglycosylation reaction can be selected form the group consisting of 2′FL, 3FL, DFL, LNFP-I, 3′SL, 6′SL, FSL, LST-a, LNT.II, LNT and LNnT.

In embodiments, HMOs which can be produced by one of the genetically modified cell and serve as acceptor oligosaccharide in the transglycosylation reaction can be selected form the group consisting of 2′FL, 3FL, LNT-II, LNT, LNnT, Para-LNnH, LNFP-I, LNFP-II, LNFP-III, LNFP-IV, LNFP-V, LNFP-VI, 3′SL, 6′SL, LST-a, and LST-c.

In embodiments, HMOs which can advantageously be produced and exported to the culture medium by one of the genetically modified cells and serve as acceptor oligosaccharide in the transglycosylation reaction can be selected form the group consisting of 2′FL, 3FL, LNT-II, LNT, LNnT, and LNFP-I.

To achieve the in situ recycling of the side-product, it is advantageous if at least one of the genetically modified cells are capable of internalizing the initial substrate used in the production of the first and/or second oligosaccharide.

In embodiments at least one of the genetically modified cells is capable of internalizing lactose, 2′FL, 3FL and/or LNT-II, depending on which compound is used as the initial substrate for making the first or second HMO. This initial substrate internalized by the cell(s) corresponds to the side-product produced by the transglycosidase reaction, which is thereby re-cycled.

In embodiments where two different genetically modified cells are co-cultured and both use lactose as substrate for producing the respective oligosaccharide, lactose will need to be fed throughout the fermentation since the transglycosidase process only produces one molecule of lactose, but two molecules of lactose are needed to produce more of the donor and acceptor oligosaccharides. If the feed of lactose is stopped towards the end of the fermentation based on a design to reach a full conversion of the acceptor oligosaccharide/HMO or higher molar ratio of the third complex HMO to the acceptor oligosaccharide/HMO, it is preferred that the transglycosidase enzyme is deactivated at the same time as the lactose feed is reduced to prevent the accumulation of a leaving group of the enzymatic step (e.g. lactose) by the side hydrolytic activity of the enzyme that triggers the reverse reaction and to maintain the designed product composition. This can be achieved by a change in pH, temperature or addition of a protease.

### Transglycosidase

Glycoside hydrolases are carbohydrate-processing enzymes in nature. Apart from hydrolysis activities, some of them also exhibit high transglycosylation activities, also called transglycosidases that catalyze the transfer of a sugar moiety between different glycosides and/or oligosaccharides.

In the contexts of the hybrid method described herein it is advantageous if the transglycosidase enzyme has as low a hydrolytic activity as possible. The hydrolytic activity of for example a transsialidase results in hydrolyses of the donor oligosaccharide, in case of 3′SL the hydrolysis reaction produces lactose and sialic acid, as well as hydrolysis of the third oligosaccharide to form the acceptor and sialic acid, e.g., in case of FSL to 3FL and sialic acid. The hydrolytic activity of for example a transfucosidase results in hydrolyses of the donor oligosaccharide, in case of 2′FL or 3FL the hydrolysis reaction produces lactose and fucose, and hydrolysis of the third oligosaccharide to form the acceptor and fucose, e.g., in case of LNFP-III to LNnT and fucose. Typically, the hydrolytic activity of the enzyme is suppressed with sufficient acceptor substrate relative to the donor substrate. However, since in the hybrid process the side hydrolytic product (e.g., lactose) is recycled back to produce the first oligosaccharide in the genetically modified cell the effect on the third HMO product formation is very low, since the transglycosidase activity will then regenerate the third oligosaccharide. Hydrolytic and transfucosylation activity of transfucosidase enzyme can for example be measured as described in Zeuner et al. 2018 Enzyme and Microbial Technology 115:37-44. Similar assays can be used for transsialidases, substituting 3FL with 3′SL or 6′SL. However, with respect to the functionality in the hybrid process described herein it is preferable to compare the hydrolytic activity of potential transglycosidases in the actual process and then assess the amount of fucose or sialic acid generated by the respective enzymes. It is desirable to use a transfucosidase that produce as little fucose as possible in the hybrid method described herein. Likewise, it is desired to us a transsialidase that produce as little sialic acid as possible in the hybrid method described herein. Fucose and sialic acid levels can for example be measured by HPLC, or alternative methods known by the person skilled in the art.

In the hybrid method described herein the transglycosidase is added to the hybrid process in an amount sufficient to mediate the transglycosylation of an acceptor oligosaccharide with a sugar moiety from a donor oligosaccharide. In the hybrid method where the sugar moiety that is transferred is either a fucosyl or a sialyl moiety the enzyme with transglycosidase activity is a transfucosidase or a transsialidase, respectively.

In the hybrid two-strain method where the sugar moiety that is transferred is either a galactose or a N-acetylglucosamine (GlcNAc) moiety the enzyme with transglycosidase activity is a trans-β-galactosidase, a trans-lacto-N-biosidase or a β-N-acetylglucosaminidase.

In embodiments, the transglycosidase enzyme is selected from the group consisting of alpha-1,2-tranfucosidase, alpha-1,3-transfucosidase, alpha-1,3/4-transfucosidase, alpha-2,3-transialidase, alpha-2,6-transsialidase, β-N-acetylglucosaminidase, trans-lacto-N-biosidase and trans-β-galactosidase.

It is advantageous if the transfucosidase is capable of using a fucosyllactose (e.g., 2′FL, 3FL or DFL) as fucosyl donor and a second oligosaccharide as acceptor. Likewise, it is advantageous if the transsialidase is capable of using a sialyllactose (3′SL or 6′SL) as sialyl donor and a second oligosaccharide as acceptor. It is advantageous if the β-1,3-N-acetylglucosaminidase is capable of using LNT-II as GlcNAc donor and a second oligosaccharide as acceptor. It is advantageous if the β-1,3-galactosidase is capable of using LNT as galactose donor and a second oligosaccharide as acceptor. It is advantageous if the trans-lacto-N-biosidase is capable of using LNT as lacto-N-biose and a second oligosaccharide as acceptor.

In embodiments where the complex oligosaccharide of at least four or five monosaccharide units produced by the hybrid method described here is an HMO, the transglycosidase has substrate specificity for an oligosaccharide acceptor which preferably is an HMO containing at least three monosaccharide units, such as four, five, six or seven monosaccharide units. In embodiments, the complex oligosaccharide has at least five monosaccharide units, such as at least 6 monosaccharide units and is a neutral non-fucosylated complex oligosaccharide.

In embodiments where the sialylated and/or a fucosylated oligosaccharide of at least four monosaccharide units produced by the hybrid method described here, is an HMO the transfucosidase or transsialidase has substrate specificity for an oligosaccharide acceptor which preferably is an HMO containing at least three monosaccharide units, such as four, five, six or seven monosaccharide units.

In embodiments the transfucosidase and/or a transsialidase has substrate specificity for at least one acceptor disaccharide or oligosaccharide selected form the group consisting of LNB, LAcNAc, 2′FL, 3FL, Lewix A, Lewis X, 2′FLacNAc, 2′FLNB, LNT, LNnT, LNH, LNnH, para-LNH, para-LNnH, LNFP-I, LNFP-II, LNFP-III, LNFP-V, LNFP-VI, LSTa and LSTc.

In further embodiments the transfucosidase and/or a transsialidase has substrate specificity for at least one HMO acceptor oligosaccharide selected form the group consisting of 2′FL, 3FL, LNT, LNnT, LNH, LNnH, para-LNH, para-LNnH, LNFP-I, LNFP-II, LNFP-III, LNFP-V, LNFP-VI, LSTa and LSTc.

One embodiment of the application is a method for producing FSL comprising the steps of:
a) cultivating a genetically modified cell producing 3FL in a culture medium; and
b) supplying 3′SL to the culture medium of a); and
c) making an enzyme with transsialidase activity available in the culture medium; and
d) incubating the 3FL, 3′SL and the transsialidase enzyme in the culture medium to form FSL and lactose; and
wherein the lactose is recycled by the cell to produce more 3FL.

Another embodiment of the application is a method for producing LNDFH-I comprising the steps of:
a) cultivating a genetically modified cell producing 3FL in a culture medium, and
b) supplying LNFP-I to the culture medium of a), and
c) making an enzyme with transfucosidase activity available in the culture medium, and
d) incubating the 3FL, LNFP-I and the transfucosidase enzyme in the culture medium to form LNDFH-I and lactose, and
wherein the lactose is recycled by the cell to produce more 3FL.

Another embodiment of the application is a method for producing LST-c comprising the steps of:
a) cultivating a genetically modified cell producing 6′SL in a culture medium, and
b) supplying LNnT to the culture medium of a), and
c) making an enzyme with transsialidase activity available in the culture medium, and
d) incubating the 6′SL, LNnT and the transsialidase enzyme in the culture medium to form LST-c and lactose, and
wherein the lactose is recycled by the cell to produce more 6′SL.

Another embodiment of the application is a method for producing LST-c and the method comprises the steps of:
a) cultivating a first genetically modified cell producing LNnT in a culture medium; and
b) supplying 6′SL to the culture medium of a); and
c) making an enzyme with transsialidase activity available in the culture medium; and
d) incubating the 6′SL, LNnT and the transsialidase enzyme in the culture medium to form LST-c and lactose; and
wherein the lactose is recycled by the cell to produce more LNnT.

Another embodiment of the application is a method for producing LNFP-III comprising the steps of:
a) cultivating a genetically modified cell producing 3FL in a culture medium, and
b) supplying LNnT to the culture medium of a), and
c) making an enzyme with transfucosidase activity available in the culture medium, and
d) incubating the 3FL, LNnT and the transfucosidase enzyme in the culture medium to form LNFP-III and lactose, and
wherein the lactose is recycled by the cell to produce more 3FL.

Another embodiment of the application is a method for producing LST-c comprising the steps of:
a) co-cultivating a first genetically modified cell growing on a first carbon source and producing 6′SL and a second genetically modified cell growing on a second carbon source and producing LNnT in a culture medium supplied with the first and second carbon source; and
b) making an enzyme with transsialidase activity available in the culture medium, and
c) incubating the 6′SL, LNnT and the transsialidase enzyme in the culture medium to form LST-c and lactose, and
wherein the lactose is recycled by the first and second genetically modified cells to produce more 6′SL and LNnT.

Another embodiment of the application is a method for producing LST-a and the method comprises the steps of:
a) cultivating a first genetically modified cell producing LNT in a culture medium; and
b) supplying 3′SL to the culture medium of a); and
c) making an enzyme with transsialidase activity available in the culture medium; and
d) incubating the 3′SL, LNT and the transsialidase enzyme in the culture medium to form LST-c and lactose; and
wherein the lactose is recycled by the cell to produce more LNT.

Another embodiment of the application is a method for producing LST-a and the method comprises the steps of:
a) cultivating a first genetically modified cell producing 3′SL in a culture medium; and
b) supplying LNT to the culture medium of a); and
c) making an enzyme with transsialidase activity available in the culture medium; and
d) incubating the 3′SL, LNT and the transsialidase enzyme in the culture medium to form LST-c and lactose; and
wherein the lactose is recycled by the cell to produce more 3′SL.

Another embodiment of the application is a method for producing LST-a comprising the steps of:
a) co-cultivating a first genetically modified cell growing on a first carbon source and producing 3′SL and a second genetically modified cell growing on a second carbon source and producing LNT in a culture medium supplied with the first and second carbon source; and
b) making an enzyme with transsialidase activity available in the culture medium, and
c) incubating the 3′SL, LNT and the transsialidase enzyme in the culture medium to form LST-a and lactose, and
wherein the lactose is recycled by the first and second genetically modified cells to produce more 3′SL and LNT.

To expand on the specific embodiments above specifying a method for producing a specific complex oligosaccharide or an oligosaccharide with a LacNAc or LNB moiety at the reducing end, Table 1 below is a non-limiting list of sialylated and/or a fucosylated oligosaccharides of at least four monosaccharide units that can potentially be obtained using different transglycosidase activities with a fucosyllactose or a sialyllactose as donor oligosaccharide and second oligosaccharide as the acceptor oligosaccharide.

TABLE 1
Non-limiting examples of complex oligosaccharides obtainable using the hybrid process

| Oligosac- | | | | | |
|---|---|---|---|---|---|
| charide | | Supplied | | | Complex |
| from | | Oligosac- | | transglycosidase | Oligosaccharide |
| strain | Function | charide | Function | activity | produced |
| 2'FL | Donor | 3FL | Acceptor | α-1,2-transfucosidase | DFL |
| DFL | | LNT | | α-1,2-transfucosidase | LNFP-I |
| | | LNFP-II | | α-1,2-transfucosidase | LNDFH-I |
| | | LNH | | α-1,2-transfucosidase | F-LNH-I |
| | | F-LNH-II | | α-1,2-transfucosidase | DF-LNH-I |
| | | Lewis-X | | α-1,2-transfucosidase | Lewis-Y |
| | | Lewis-A | | α-1,2-transfucosidase | Lewis-B |
| 3FL | Donor | LNT | Acceptor | α-1,3/4-transfucosidase | LNFP-II |
| DFL | | LNT | | α-1,3-transfucosidase | LNFP-V |
| | | | | (Glc specific) | |
| | | 2FL | | α-1,3-transfucosidase | DFL |
| | | LNH | | α-1,3-transfucosidase | F-LNH-II |
| | | | | (Glc specific) | |
| | | F-LNH-I | | α-1,3-transfucosidase | DF-LNH-I |
| | | F-LNH-II | | α-1,4-transfucosidase | DF-LNH-II |
| | | LNnT | | α-1,3-transfucosidase | LNFP-III |
| | | | | (GlcNAc specific) | |
| | | LNnT | | α-1,3-transfucosidase | LNFP-IV |
| | | | | (Glc specific) | |
| | | LNFP-I | | α-1,4-transfucosidase | LNDFH-I |
| | | LNFP-V | | α-1,4-transfucosidase | LNDFH-II |
| | | LNFP-VI | | α-1,3-transfucosidase | LNDFH-III |
| | | | | (GlcNAc specific) | |
| | | LNFP-III | | α-1,3-transfucosidase | LNDFH-III |
| | | LNFP-II | | α-1,3-transfucosidase | LNDFH-II |
| | | LNnH | | α-1,3-transfucosidase | DF-LNnH |
| | | | | (GlcNAc specific) | |
| | | Para-LNnH | | α-1,3-transfucosidase | DF-para-LNnH |
| | | | | (GlcNAc specific) | |
| | | Para-LNH | | α-1,3/4-transfucosidase | DF-para-LNH |
| | | F-LNH-I | | α-1,3/4-transfucosidase | TF-LNH |
| | | 2'-FLacNAc | | α-1,3/4-transfucosidase | Lewis-Y |
| | | 2'-FLNB | | α-1,3/4-transfucosidase | Lewis-B |
| | | LacNAc | | α-1,3/4-transfucosidase | Lewis-X |
| | | LNB | | α-1,3/4-transfucosidase | Lewis-A |
| 3'SL | Donor | LNFP-II | Acceptor | α-2,3-transsialidase | FLSTa |
| | | | | | (S-LNFP-II) |
| 3'SL | | 3FL | | α-2,3-transsialidase | FSL |
| | | LNT | | α-2,3-transsialidase | LSTa |
| | | LNFP-I | | α-2,3-transsialidase | FLSTa |
| | | F-LNH-I | | α-2,3-transsialidase | FS-LNH |
| | | LSTb | | α-2,3-transsialidase | DSLNT |
| | | LacNAc | | α-2,3-transsialidase | 3'SLacNAc |
| | | LNB | | α-2,3-transsialidase | 3'SLNB |
| 6'SL | Donor | LNnT | Acceptor | α-2,6-transsialidase | LSTc |
| | | LNFP-III | | α-2,6-transsialidase | FLSTc |
| | | LacNAc | | α-2,6-transsialidase | 6'SLacNAc |
| | | LNB | | α-2,6-transsialidase | 6'SLNB |
| | | LNFP-I | | α-2,6-transsialidase | FLSTb |
| | | | | (GlcNAc specific) | (S-LNFP-I) |
| | | LNT | | α-2,6-transsialidase | LSTb |
| | | | | (GlcNAc specific) | |
| | | LSTa | | α-2,6-transsialidase | DSLNT |
| | | | | (GlcNAc specific) | |
| LNT-II | Donor | 3'FL | Acceptor | β-1,3-N- | GlcNAc(1-3)-3FL |
| | | | | acetylglucosaminidase | |
| LNT | Donor | LNnT | Acceptor | β-1,3-galactosidase | gal-LNnT |
| LNT | Donor | LNnT | Acceptor | Trans-lacto-N-biosidase | pLNH |
| 3'SLacNAc | Donor | Lewis-X | Acceptor | α-2,3-transsialidase | Sialyl-Lewis X |
| 3'SLNB | Donor | Lewis-A | Acceptor | α-2,3-transsialidase | Sialyl-Lewis A |

An example of a trans-lacto-N-biosidase from B. longum JCM1217 (LnbX, Sakamura et al. J. Biol. Chem. 288, 25194 (2013), GenBank nr. DAA64542) and its truncated functional analogs can be utilized to make linear lacto-N-biose containing oligosaccharides. In one embodiment variants with 70% identity to the sequence from amino acid position 45 to 625 of GenBank nr. DAA64542 with a mutation in at least at one or more of amino acid positions selected from 410, 416, 439 and 442, said amino acid numbering being according to GenBank nr. DAA64542 are used to transfer lacto-N-biose moieties from a donor oligosaccharide to an acceptor oligosaccharide. Advantages variants are described in PA202201151 where they are showed to function in an in vitro process generating pLNH from LNT and LNnT.

Further, Non-limiting examples of relevant transsialidases and transfucosidases are shown in tables 4 and 5 below.

### Transsialidase

Enzymes having transsialidase activity and which are suitable for the purpose of the method of making sialylated oligosaccharides with the hybrid process described herein, can be selected from sialidase and transsialidase enzymes.

Sialidases or neuraminidase (EC 3.2.1.18) and trans-sialidases (EC 2.4.1.-), both classify in the GH33 family as defined by the CAZY nomenclature (http://www.cazy.org), as enzymes with the ability of hydrolyzing the alpha-linkage of the terminal sialic acid (exo-α-sialidase), bound to galactose or glucose with an alpha-2,3 or an alpha-2,6 linkage, of various sialylglycoconjugates. The enzymes are found particularly in diverse virus families and bacteria, and also in protozoa, some invertebrates and mammals. Sialidases, are despite the hydrolytic activity, capable of acting as a catalyst for a transsialylation reaction due to their transsialidase activity with alpha-2,3 and/or alpha-2,6 selectivity.

In order to improve transsialidase activity of the sialidases, they may be subjected to alteration by various engineering techniques. Preferably, under the conditions in the hybrid method described herein, the formation of sialic acid is low. Preferably, the amount of sialic acid is below 5% of the total molar % of the donor oligosaccharide and the third oligosaccharide, more preferably below 3% of the total molar % of the donor oligosaccharide and the third oligosaccharide. WO2012/007588 describe a series of suitable transsialidases.

TABLE 2
Suitable transsialidases

| GI number in | | SEQ |
|---|---|---|
| GenBank | | ID |
| Database | Organisms | NO: |
| gi\|213524659 | Bifidobacterium longum subsp. | 4 |
| | infantis ATCC 15697 | |
| gi\|213523006 | | 5 |
| gi\|309252191 | Bifidobacterium bifidum S17 | 6 |
| gi\|309252190 | | 7 |
| gi\|310867437 | Bifidobacterium bifidum PRL2010 | 8 |
| gi\|310867438 | | 9 |
| gi\|224283484 | Bifidobacterium bifidum NCIMB 4 1171 | 10 |
| gi\|224283485 | | 11 |
| gi\|334283443 | Bifidobacterium bifidum JCM1254 | 12 |
| gi\|840708 | Trypanosoma cruzi | 13 |
| gi\|432485 | | 14 |
| — | Variant of gi\|432485 | 60 |
| gi\|267844791 | Photobacterium leiognathi | 40 |
| — | N-terminal truncated variant of gi\|267844791 | 41 |

In embodiments the transsialidase is selected from the group of the suitable transsialidase enzymes in table 2 or a functional homologue thereof having an amino acid sequence of at least 70% identity, such as at least 80%, such as at least 85%, such as at least 90%, such at least 95% or even 97%, 98% or 99% identity compared to an individual transsialidase sequence in table 2.

In one embodiment the transfucosidase comprises or consist of an amino acid sequence of SEQ ID NO: 13, 14, 40, 41 or 60.

In embodiments where the transsialidase is added to the fermentation broth it is sterile filtered before it is introduced into the hybrid process. The transsialidase is added to the hybrid process in an activity sufficient to mediate the transsialylation of the acceptor oligosaccharide with the donor oligosaccharide.

In alternative embodiments, the genetically modified cell capable of producing the first oligosaccharide is further modified by introducing a heterologous nucleic acid which encodes a transsialidase. Preferably, the transsialidase is secreted/exported into the culture medium by the further genetically modified cell. The heterologous nucleic acid encoding the transsialidase may be expressed from an inducible promoter, such that the expression of the transsialidase is delayed compared to the formation of the first oligosaccharide produced by the same cell. The advantage of having delayed expression of the transsialidase is that the first oligosaccharide will not become rate limiting in the enzymatic step of the hybrid process.

### Transfucosidases

Enzymes having transfucosidase activity and which are suitable for the purpose of the method of making fucosylated oligosaccharides with the hybrid process described herein, can be selected from fucosidase and transfucosidase enzymes.

Alpha-L-fucosidases are classified according to EC 3.2.1.38 and EC 3.2.1.51 and belong to the glycoside hydrolases families 29 and 95 (GH29 and GH95) as defined by the CAZY nomenclature (http://www.cazy.org). The substrate specificity of the GH29 family is broad whereas that of the GH95 family has strict specificity to alpha-1,2-linked fucosyl residues. The GH29 family seems to be divided into two subfamilies. One subfamily typically has strict specificity towards alpha-1,3- and alpha-1,4-fucosidic linkages. The members of a further subfamily have broader specificity, covering two or three alpha-fucosyl linkages. Alpha-L-fucosidases generally hydrolyse the terminal fucosyl residue from glycans. These enzymes are also capable to act as catalyst for a fucosylation reaction due to their transfucosylation activity and thus may be used in the context of the hybrid method described herein.

In order to improve transfucosidase activity of the fucosidases may be subjected to alteration by various engineering techniques. WO2016/063261 and Zeuner et al (2018 Enzyme and Microbial Technology 115:37-44) describes mutants of an alpha-1-3/4 transfucosidase from Bifidobacterium longum subsp. infants ATCC 15697 (NBI accession No. WP_012578573) or Bifidobacterium bifidum JCM 1254 (GenBank BAH80310.1), which have increased transfucosidase activity and reduced hydrolase activity Preferably, under the conditions in the hybrid method described herein, the formation of fucose is low. Preferably, the amount of fucose is below 5% of the total molar % of the donor oligosaccharide and the third oligosaccharide, more preferably below 3% of the total molar % of the donor oligosaccharide and the third oligosaccharide.

TABLE 3
Suitable transfucosidases

| GI number | | SEQ |
|---|---|---|
| in GenBank | | ID |
| Database | Organisms | NO: |
| gi\|4980806 | Thermotoga maritima MSB8 | 15 |
| gi\|13816464 | Sulfolobus solfataricus P2 | 16 |
| gi\|34451973 | Bifidobacterium bifidum JCM 1254 | 17 |
| gi\|242345155 | Bifidobacterium bifidum JCM 1254 | 18 |
| — | Variant of gi\|242345155 | 19 |
| gi\|213524647 | Bifidobacterium longum subsp. | 20 |
| | infantis ATCC 15697 | |
| gi\|213522629 | Bifidobacterium longum subsp. | 21 |
| | infantis ATCC 15697 | |
| gi\|213522799 | Bifidobacterium longum subsp. | 22 |
| | infantis ATCC 15697 | |
| gi\|213524646 | Bifidobacterium longum subsp. | 23 |
| | infantis ATCC 15697 | |
| gi\|501574167 | Bifidobacterium longum subsp. | 24 |
| | infantis ATCC 15697 | |
| gi\|320457227 | Bifidobacterium longum subsp. | 25 |
| | infantis ATCC 15697 | |
| gi\|320457408 | Bifidobacterium longum subsp. | 26 |
| | infantis ATCC 15697 | |
| gi\|320459369 | Bifidobacterium longum subsp. | 27 |
| | infantis ATCC 15697 | |
| gi\|320459368 | Bifidobacterium longum subsp. | 28 |
| | infantis ATCC 15697 | |
| gi\|501574157 | Bifidobacterium longum | 29 |
| — | W135E, A174F Variant of gi\|501574157 | 30 |
| gi\|310867039 | Bifidobacterium bifidum PRL2010 | 31 |
| gi\|310865953 | Bifidobacterium bifidum PRL2010 | 32 |
| gi\|309251774 | Bifidobacterium bifidum S17 | 33 |
| gi\|296182927 | Bifidobacterium longum subsp | 34 |
| | longum JDM 301 | |
| gi\|283103603 | Bifidobacterium dentium Bd1 | 35 |
| gi\|190713109 | Lactobacillus casei BL23 | 36 |
| gi\|190713871 | Lactobacillus casei BL23 | 37 |
| gi\|190713978 | Lactobacillus casei BL23 | 38 |
| — | S168E, A174H, E413R Variant of gi\|501574157 | 39 |
| — | W135F, A174N, N274A, E413R Variant of | 59 |
| | gi\|501574157 | |

In embodiments the transfucosidase is selected from the group of the suitable transfucosidase enzymes in table 3 or functional homologues thereof having an amino acid sequence of at least 70% identity, such as at least 80%, such as at least 85%, such as at least 90%, such at least 95% or even 97%, 98% or 99% identity compared to an individual transfucosidase sequence in table 3.

In embodiments the transfucosidase enzyme originates from Bifidobacterium bifidum or Bifidobacterium longum.

In one embodiment the transfucosidase comprises or consist of an amino acid sequence of SEQ ID NO: 19, 30, 39 or 59.

In embodiments the transfucosidase is added to the hybrid method it is sterile filtered before it is introduced into the hybrid process. The transfucosidase is added to the hybrid process in an activity sufficient to mediate the transfucosylation of the acceptor oligosaccharide with the donor oligosaccharide.

In alternative embodiments the genetically modified cell capable of producing the first oligosaccharide is further modified by introducing a heterologous nucleic acid which encodes a transfucosidase. Preferably, the transfucosidase is secreted/exported into the culture medium by the further genetically modified cell. The heterologous nucleic acid encoding the transfucosidase may be expressed from an inducible promoter, such that the expression of the transfucosidase is delayed compared to the formation of the first oligosaccharide produced by the same cell. The advantage of having delayed expression of the transfucosidase is that the first oligosaccharide will not become rate limiting in the enzymatic step of the hybrid process.

### The Genetically Modified Cell

In the present context, the terms “a genetically modified cell” and “a genetically engineered cell” are used interchangeably. As used herein “a genetically modified cell” is a host cell whose genetic material has been altered by human intervention using a genetic engineering technique, such a technique is e.g., but not limited to transformation or transfection e.g., with a heterologous polynucleotide sequence, Crisper/Cas editing and/or random mutagenesis. In one embodiment the genetically engineered cell has been transformed or transfected with a recombinant nucleic acid sequence.

The genetic modifications can e.g., be selected from inclusion of glycosyltransferases, transglycosidases, and/or metabolic pathway engineering and inclusion of transporter proteins, including importer and exporters as described in the present application, all of which the skilled person will know how to combine into a genetically modified cell capable of producing the desired HMO.

The genetically engineered cell is preferably a microbial cell, such as a prokaryotic cell or eukaryotic cell. Appropriate microbial cells that may function as a host cell include bacterial cells, archaebacterial cells, algae cells and fungal cells.

The genetically engineered cell may be e.g., a bacterial or yeast cell. In one preferred embodiment, the genetically engineered cell is a bacterial cell.

### Host Cells

Regarding the bacterial host cells, there are, in principle, no limitations, they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the invention could be Erwinia herbicola (Pantoea agglomerans), Citrobacter freundii, Campylobacter sp, Corynebacterium sp, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species for the invention described herein. Also included as part of this invention are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of a heterologous product are e.g., yeast cells of the genera Komagataella, Kluyveromyces, Yarrowia, Pichia, Saccaromyces, Schizosaccharomyces or Hansenula or from a filamentous fungus of the genera Aspargillus, Fusarium or Thricoderma. More specifically yeast cell species such as Komagataella phaffii, Kluyveromyces lactis, Yarrowia lipolytica, Pichia pastoris, and Saccaromyces cerevisiae or filamentous fungi species such as A. niger, A. nidulans, A. oryzae, F. solani, F. graminearum and T. reesei.

In one or more exemplary embodiments, the genetically engineered cell is selected from the group consisting of Escherichia sp., Bacillus sp., Lactobacillus sp., Corynebacterium sp. and Campylobacter sp.

In one or more exemplary embodiments, the genetically engineered cell is selected from the group consisting of of Escherichia coli, Bacillus subtilis, Lactobacillus lactis, Corynebacterium glutamicum, Yarrowia lipolytica, Pichia pastoris, and Saccharomyces cerevisiae.

In one or more exemplary embodiments, the genetically engineered cell is B. subtilis.

In one or more exemplary embodiments, the genetically engineered cell is S. cerevisiae or P. pastoris.

In one or more exemplary embodiments, the genetically engineered cell is Escherichia coli.

In one or more exemplary embodiments, the invention relates to a genetically engineered cell, wherein the cell is derived from the E. coli K-12 strain or DE3.

### Glycosyltransferases

The genetically modified cell according to the present invention comprises at least one recombinant nucleic acid sequence encoding at least one glycosyltransferase capable of transferring a glycosyl residue from a glycosyl-donor to an acceptor oligosaccharide to synthesize an oligosaccharide product, such as a human milk oligosaccharide product. The nucleic acid sequence encoding the one or more expressed glycosyltransferase(s) may be integrated into the genome (by chromosomal integration) of the genetically engineered cell, or alternatively, it may be comprised in a plasmid and expressed as plasmid-borne, as described in the present disclosure.

The genetically modified cell according to the present invention may comprise at least two recombinant nucleic acid sequences encoding two different glycosyltransferases capable of transferring a glycosyl residue from a glycosyl-donor to an acceptor oligosaccharide.

The one or more glycosyltransferase is preferably selected from the group of enzymes having the activity of an α-1,2-fucosyltransferase, α-1,3-fucosyltransferase, α-1,3/4-fucosyltransferase, α-1,4-fucosyltransferase α-2,3-sialyltransferase, α-2,6-sialyltransferase, β-1,3-N-acetylglucosaminyltransferase, β-1,6-N-acetylglucosaminyltransferase, β-1,3-galactosyltransferase and β-1,4-galactosyltransferase, described in more detail below.

### Beta-1,3-N-acetyl-glucosaminyltransferase

A β-1,3-N-acetyl-glucosaminyltransferase is any protein which comprises the ability of transferring the N-acetyl-glucosamine of UDP-N-acetyl-glucosamine to lactose or another acceptor molecule, in a beta-1,3-linkage. Preferably, a β-1,3-N-acetyl-glucosaminyltransferase used herein does not originate in the species of the genetically engineered cell i.e., the gene encoding the β-1,3-galactosyltransferase is of heterologous origin. Non-limiting examples of β-1,3-N-acetyl-glucosaminyltransferase are given in table 4. β-1,3-N-acetyl-glucosaminyltransferase variants may also be useful, preferably such variants are at least 80%, such as at least 85%, such as at least 90, such as at least 95% identical to one of the β-1,3-N-acetyl-glucosaminyltransferase in table 4.

TABLE 4
List of β-1,3-N-acetyl-glucosaminyltransferase

| Protein Name | GenBank ID | origin |
|---|---|---|
| IgtA | WP_033911473.1 | Neisseria meningitidis |
| | WP_002248149 | |
| | AAF42258.1 | |
| | ELK60643.1 | |
| IgtA | AAK70338.1 | Neisseria gonorrhoeae |
| | ACF31229.1 | |
| IgtA | AAK02595.1 | Pasteurella multocida |
| IgtA | EEZ72046.1 | Neisseria cinerea |
| HD0466 | WP_010944479.1 | Haemophilus ducreyi |
| PmnagT | WP_014390683.1 | Pasteurella multocida |

In embodiments the glycosyltransferase encoded by the genetically engineered cell is an β-1,3-N-acetyl-glucosaminyltransferase from table 6. Preferably, the glycosyltransferase in the genetically engineered cell is a β-1,3-N-acetyl-glucosaminyltransferase from Neisseria meningitidis, such as the β-1,3-N-acetyl-glucosaminyltransferase of SEQ ID NO: 45 or a functional variant thereof.

### β-1,3-galactosyltransferase

A β-1,3-Galactosyltransferase is any protein that comprises the ability of transferring the galactose of UDP-Galactose to a N-acetyl-glucosaminyl moiety to an acceptor molecule in a beta-1,3-linkage. Preferably, a β-1,3-galactosyltransferase used herein does not originate in the species of the genetically engineered cell i.e., the gene encoding the β-1,3-galactosyltransferase is of heterologous origin. Non-limiting examples of β-1,3-galactosyltransferases are given in table 5. β-1,3-galactosyltransferases variants may also be useful, preferably such variants are at least 80%, such as at least 85%, such as at least 90, such as at least 95% identical to one of the β-1,3-galactosyltransferases in table 5.

TABLE 5
List of beta-1,3-glycosyltransferases

| Protein | | |
|---|---|---|
| Name | GenBank ID | Origin |
| WbgO | WP_000582563.1 | E. coli 055:H7 |
| cpsIBJ | AB050723.1 | Streptococcus agalactiae type lb OI2 |
| Jhp0563 | AEZ55696.1 | Helicobacter pylori |
| GalTK | WP_111735921.1 | Helicobacter pylori ATCC 43504 |
| CvB3GalT | WP_080969100.1 | Chromobacterium violaceum |

In embodiments the at least one glycosyltransferase encoded by the genetically engineered cell is β-1,3-N-acetylglucosaminyltransferase and a β-1,3-galactosyltransferase. Preferably, the glycosyltransferase in the genetically engineered cell is the β-1,3-N-acetylglucosaminyltransferase is selected from table 4 and the β-1,3-galactosyltransferase is selected from table 5. Even more preferred the 1,3-N-acetylglucosaminyltransferase is from a Neisseria sp. and the β-1,3-galactosyltransferase is from Helicobacter pylori, such as the β-1,3-N-acetylglucosaminyltransferase with GenBank ref nr. WP_002248149.1 and the β-1,3-galactosyltransferase with GenBank ref nr. WP_111735921.1.

### β-1,4-galactosyltransferase

A β-1,4-Galactosyltransferase is any protein that comprises the ability of transferring the galactose of UDP-Galactose to a N-acetyl-glucosaminyl moiety. Preferably, a β-1,4-galactosyltransferase used herein does not originate in the species of the genetically engineered cell i.e., the gene encoding the β-1,4-galactosyltransferase is of heterologous origin. Non-limiting examples of β-1,4-galactosyltransferases are given in table 6. β-1,4-galactosyltransferases variants may also be useful, preferably such variants are at least 80%, such as at least 85%, such as at least 90, such as at least 95% identical to one of the β-1,4-galactosyltransferases in table 6.

TABLE 6
List of beta-1,4-glycosyltransferases

| Protein Name | GenBank ID | Origin |
|---|---|---|
| GalT | WP_001262061.1 | Helicobacter pylori |
| LgtB | AAF42257.1 | Neisseria meningitidis MC58 |

In embodiments the at least one glycosyltransferase encoded by the genetically engineered cell is β-1,3-N-acetylglucosaminyltransferase and a β-1,4-galactosyltransferase. Preferably, the glycosyltransferase in the genetically engineered cell is the β-1,3-N-acetylglucosaminyltransferase is selected from table 4 and the β-1,4-galactosyltransferase is selected from table 6. Even more preferred the 1,3-N-acetylglucosaminyltransferase is from a Neisseria sp. and the β-1,4-galactosyltransferase is from Helicobacter pylori, such as the β-1,3-N-acetylglucosaminyltransferase with GenBank ref nr. WP_002248149.1 and the β-1,4-galactosyltransferase with GenBank ref nr. WP_001262061.1 or SEQ ID NO: 46, or a functional variant thereof.

### Alpha-1,2-fucosyltransferase

An α-1,2-fucosyltransferase is a protein that comprises the ability to catalyze the transfer of fucose from a donor substrate, for example, GDP-fucose, to an acceptor molecule in an alpha-1,2-linkage. Preferably, an alpha-1, 2-fucosyltransferase used herein does not originate in the species of the genetically engineered cell i.e., the gene encoding the alpha-1, 2-fucosyltransferase is of heterologous origin. Non-limiting examples of alpha-1,2-fucosyltransferase are given in table 7. Alpha-1,2-fucosyltransferase variants may also be useful, preferably such variants are at least 80%, such as at least 85%, such as at least 90, such as at least 95% identical to one of the alpha-1,2-fucosyltransferase in table 7.

TABLE 7
List of α-1,2-fucosyltransferase

| Protein | | |
|---|---|---|
| Name | GenBank ID | Origin |
| FutC | WP_080473865.1 | Helicobacter pylori |
| | | ATCC 26695 |
| wbgL | ADN43847 | E. coli 0126 |
| Smob | WP_126455392.1 | Sulfuriflexus mobilis |
| FucT2_HpUA802 | AAC99764.1 | Helicobacter pylori |
| FucT2_EcO126t | ABE98421.1 | Escherichia coli |
| FucT2_Hm12198 | CBG40460.1 | Helicobacter mustelae |
| | | 12198 |
| FucT2 Pm9515 | ABM71599.1 | Brochlorococcus marinus |
| | | str. MIT 9515 |
| FucT2_HpF57 | BAJ59215.1 | Helicobacter pylori F57 |
| FucT54 | ADE13114.1 | Sideroxydans lithotrophicus |
| | | ES-11 |
| Mtun | WP_031437198.1 | Methylobacter tundripaludum |

In embodiments the glycosyltransferase encoded by the genetically engineered cell is an α-1,2-fucosyltransferase from table 7. Preferably, the glycosyltransferase in the α-1,2-fucosyltransferase is from Helicobacter pylori, such as the α-1,2-fucosyltransferase with the GenBank accession nr. WP_080473865.1.

### Alpha-1,3-fucosyltranferase

An alpha-1,3-fucosyltranferase refer to a glycosyltransferase that catalyzes the transfer of fucose from a donor substrate for example, GDP-fucose, to an acceptor molecule in an alpha-1,3-linkage. Preferably, an alpha-1,3-fucosyltransferase used herein does not originate in the species of the genetically engineered cell i.e., the gene encoding the alpha-1,3-fucosyltransferase is of heterologous origin. Non-limiting examples of alpha-1,3-fucosyltransferase are given in table 8. Alpha-1,3-fucosyltransferase variants may also be useful, preferably such variants are at least 80%, such as at least 85%, such as at least 90, such as at least 95% identical to one of the alpha-1,3-fucosyltransferase in table 8.

TABLE 8
List of α-1,3-fucosyltransferase

| Protein Name | GenBank ID | Origin |
|---|---|---|
| FucT | AAB81031.1 | Helicobacter pylori |
| | | NCTC 11639 |
| FutA | WP_000487428.1 | Helicobacter pylori |
| | | ATCC 26695 |
| CafC | WP_007483358.1 | Bacteroides nordii |
| FucT6_3_Bf | CAH09151.1 | Bacteroides fragilis |
| | | NCTC 9343 |
| FucT7_3_Bf | CAH09495.1 | Bacteroides fragilis |
| | | NCTC 9343 |
| FucT_3_Am | ACD04596.1 | Akkermansia muciniphila |
| | | ATCC BAA-835 |
| MAMA_R764 | AGC02224.1 | Acanthamoeba polyphaga |
| | | moumouvirus |
| Mg791 | AEQ33441.1 | Megavirus chiliensis |
| Moumou_00703 | YP_007354660.1 | Acanthamoeba polyphaga |
| | | moumouvirus |

In embodiments the glycosyltransferase encoded by the genetically engineered cell is an α-1,3-fucosyltransferase from table 8. Preferably, the glycosyltransferase in the genetically engineered cell is the α-1,3-fucosyltransferase from Helicobacter pylori, such as the α-1,3-fucosyltransferase of SEQ ID NO: 1 or a functional homologue thereof.

### Alpha-1,3/4-fucosyltransferase

An alpha-1,3/4-fucosyltransferase refer to a glycosyltransferase that catalyzes the transfer of fucose from a donor substrate for example, GDP-fucose, to an acceptor molecule in an alpha-1,3- or alpha 1,4-linkage. Preferably, an alpha-1,3/4-fucosyltransferase used herein does not originate in the species of the genetically engineered cell i.e., the gene encoding the alpha-1,3/4-fucosyltransferase is of heterologous origin. Non-limiting examples of alpha-1,3/4-fucosyltransferase are given in table 9. alpha-1,3/4-fucosyltransferase variants may also be useful, preferably such variants are at least 80%, such as at least 85%, such as at least 90%, such as at least 95% identical to one of the alpha-1,3/4-fucosyltransferase in table 9.

TABLE 9
List of α-1,3/4-fucosyltransferase

| Protein | | |
|---|---|---|
| Name | GenBank ID | Origin |
| FucTIII | AY450598.1 | Helicobacter pylori strain DSM 6709 |
| FutA | WP_000487428.1 | Helicobacter pylori ATCC 26695 |

In embodiments the glycosyltransferase encoded by the genetically engineered cell is an α-1,3/4-fucosyltransferase from table 11. Preferably, the glycosyltransferase in the genetically engineered cell is the α-1,3/4-fucosyltransferase FutA from Helicobacter pylori, such as the α-1,3/4-fucosyltransferase of SEQ ID NO: 1 or a functional variant thereof.

### Alpha-2,3-sialyltransferase

An α-2,3-sialyltransferase refer to a glycosyltransferase that catalyzes the transfer of sialyl from a donor substrate for example, CMP-N-acetylneuraminic acid, to an acceptor molecule in an alpha-2,3-linkage. Preferably, an alpha-2,3-sialyltransferase used herein does not originate in the species of the genetically engineered cell i.e., the gene encoding the 2,3-sialyltransferase is of heterologous origin. Non-limiting examples α-2,3-sialyltransferase are given in table 10. α-2,3-sialyltransferase variants may also be useful, preferably such variants are at least 80%, such as at least 85%, such as at least 90, such as at least 95% identical to one of the α-2,3-sialyltransferase in table 10.

TABLE 10
List of α-2,3-sialyltransferase

| Protein | | |
|---|---|---|
| Name | GenBank ID | Origin |
| Avi | WP_115249238.1 | Avibacterium avium |
| Ccol | WP_075498955.1 | Campylobacter coli |
| Ccol2 | EAH6554614.1 | Campylobacter coli |
| Chepa | WP_066776435.1 | Campylobacter hepaticus |
| Cinf1 | WP_011272254.1 | Haemophilus influenzae |
| Cjej1 | EBD1936710.1 | Campylobacter jejuni |
| Clari1 | EGK8106227.1 | Campylobacter lari |
| CstI | AAF13495.1 | Campylobacter jejuni |
| CstII | AAF31771.1 | Campylobacter jejuni |
| Csub1 | WP_039664428.1 | Campylobacter subantarcticus |
| MhnNBse | WP_176810284.1 | Mannheimia (multispecies) |
| Neigon | AAW89748.1 | Neisseria gonorrhoeae FA 1090 |
| Nst | AAC44541.1 | Neisseria meningitidis MC58 |
| PM70 | AAK03258.1 | Pasteurella multocida subsp. multocida |
| | | str. Pm70 |
| PmN | WP_005726268.1 | Pasteurella (multispecies) |
| Pmult | WP_005753497.1 | Pasteurela multocida |
| Poral | WP_101774487.1 | Pasteurella oralis |
| Poral_2 | WP_101774701.1 | Pasteurella oralis |

In embodiments the glycosyltransferase encoded by the genetically engineered cell is an α-2,3-sialyltransferase from table 10. Preferably, the glycosyltransferase in the genetically engineered cell is a α-2,3-sialyltransferase from Campylobacter lari, Neisseria meningitidis or Pasteurella oralis, such as the α-2,3-sialyltransferase with the GenBank accession nr. EGK8106227.1, AAC44541.1, or WP_101774487.1.

### Alpha-2,6-sialyltransferase

An alpha-2,6-sialyltransferase refer to a glycosyltransferase that catalyzes the transfer of sialyl from a donor substrate for example, CMP-N-acetylneuraminic acid, to an acceptor molecule in an alpha-2,6-linkage. Preferably, an alpha-2,6-sialyltransferase used herein does not originate in the species of the genetically engineered cell i.e., the gene encoding the 2,6-sialyltransferase is of heterologous origin. Non-limiting examples α-2,6-sialyltransferase are given in table 11. α-2,6-sialyltransferase variants may also be useful, preferably such variants are at least 80%, such as at least 85%, such as at least 90, such as at least 95% identical to one of the α-2,6-sialyltransferase in table 11.

TABLE 11
List of α-2,6-sialyltransferase

| Protein Name | GenBank ID | Origin |
|---|---|---|
| HAC1268 | STP03859.1 | Helicobacter acinonychis str. Sheeba |
| Pts6-119 | AB500947.1 | Photobacterium leiognathi JT-SHIZ-119 |
| Pts6-224 | BAF92026.1 | Photobacterium sp JT-ISH-224 |
| PiST6_145 | BAF91416.1 | Photobacterium leiognathi |

In embodiments the glycosyltransferase encoded by the genetically engineered cell is α-2,6-sialyltransferase from table 11. the glycosyltransferase in the genetically engineered cell is a α-2,6-sialyltransferase from Photobacterium sp, such as the α-2,6-sialyltransferase with the GenBank accession nr. AB500947.1 or BAF92026.1 or the α-2,6-sialyltransferase of SEQ ID NO: 43 or a functional variant thereof.

### Nucleotide-Activated Sugar Pathways

In the genetically engineered cell used in the hybrid method described herein a glycosyltransferase mediated glycosylation reaction takes place inside the cell, in which an activated sugar nucleotide serves as glycosyl-donor. An activated sugar nucleotide generally has a phosphorylated glycosyl residue attached to a nucleoside. A specific glycosyl transferase enzyme accepts only a specific sugar nucleotide. Thus, preferably the following activated sugar nucleotides are involved in the glycosyl transfer: glucose-UDP-GlcNAc, UDP-galactose, UDP-glucose, UDP-N-acetylglucosamine, UDP-N-acetylgalactosamine (GlcNAc) and CMP-N-acetylneuraminic acid. The genetically modified cell according to the present invention can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid (CMP-Neu5Ac).

In embodiments, the genetically modified cell is capable of producing one or more activated sugar nucleotides mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, sucrose, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)).

The enzymes involved in the de novo biosynthetic pathway of an activated sugar nucleotide can be naturally present in the cell or introduced into the cell by means of gene technology or recombinant DNA techniques, all of them are part of the general knowledge of the skilled person.

In embodiments the pathway to produce a nucleotide-activated sugar is the de novo GDP-fucose pathway (gmd, wcaG, manB, manC and manA) and/or the sialic acid sugar nucleotide pathway (neuB, neuC and neuA) as described below.

In another embodiment, the genetically modified cell can utilize salvaged monosaccharides for sugar nucleotide synthesis. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the host cell.

### Colanic Acid Gene Cluster

For the production of fucosylated oligosaccharides/HMOs the colanic acid gene cluster is important to ensure presence of sufficient GDP-fucose. In Escherichia coli GDP-fucose is an intermediate in the production of the extracellular polysaccharide colanic acid, a major oligosaccharide of the bacterial cell wall. In the context of the present invention the colanic acid gene cluster encodes most of the enzymes involved in the de novo synthesis of GDP-fucose (gmd, wcaG, wcaH, wcaI, manB, manC), whereas one or several of the genes downstream of GDP-L-fucose, such as wcaJ, can be deleted to prevent conversion of GDP-fucose to colanic acid.

The de novo GDP-fucose pathway genes responsible for the formation of GDP-fucose comprises or consists of the following genes: i) manA which encodes the protein mannose-6 phosphate isomerase (EC 5.3.1.8, UniProt accession nr. P00946), which facilitates the interconversion of fructose 6-phosphate (F6P) and mannose-6-phosphate; ii) manB which encodes the protein phosphomannomutase (EC 5.4.2.8, UniProt accession nr P24175), which is involved in the biosynthesis of GDP-mannose by catalyzing conversion mannose-6-phosphate into mannose-1-phosphate; iii) manC which encodes the protein mannose-1-phosphate guanylyltransferase guanylyltransferase (EC:2.7.7.13, UniProt accession nr P24174), which is involved in the biosynthesis of GDP-mannose through synthesis of GDP-mannose from GTP and α-D-mannose-1-phosphate; iv) gmd which encodes the protein GDP-mannose-4,6-dehydratase (UniProt accession nr P0AC88), which catalyzes the conversion of GDP-mannose to GDP-4-dehydro-6-deoxy-D-mannose; v) wcaG (fcl) which encodes the protein GDP-L-fucose synthase (EC 1.1.1.271, UniProt accession nr P32055) which catalyses the two-step NADP-dependent conversion of GDP-4-dehydro-6-deoxy-D-mannose to GDP-fucose.

Accordingly, it is preferred that the genetically engineered cell, when producing one or more fucosylated heterologous products, overexpresses either the entire colonic acid gene cluster and/or one or more genes of the de novo GDP-fucose pathway selected from the group consisting of manA, manB, manC, gmd and wcaG.

In one or more exemplary embodiment(s), the colanic acid gene cluster responsible for the formation of GDP-fucose may be expressed from its native genomic locus. The expression may be actively modulated to increase GDP-fucose formation. The expression can be modulated by swapping the native promoter with a promoter of interest, and/or increasing the copy number of the colanic acid genes coding said protein(s) by expressing the gene cluster from another genomic locus than the native, or episomally expressing the colanic acid gene cluster or specific genes thereof.

In relation to the present disclosure, the term “native genomic locus”, in relation to the colanic acid gene cluster, relates to the original and natural position of the gene cluster in the genome of the genetically engineered cell.

### Sialic Acid Sugar Nucleotide Synthesis Pathway

If the genetically modified cell is to produce a sialylated oligosaccharide/HMO the genetically modified cell comprises a sialic acid sugar nucleotide synthesis capability, i.e., the genetically modified cell comprises a biosynthetic pathway for making a sialate sugar nucleotide, such as CMP-N-acetylneuraminic acid as glycosyl-donor for the sialyltransferases. E.g., the genetically modified cell comprises a sialic acid synthetic capability through provision of an exogenous UDP-GlcNAc 2-epimerase (e.g., neuC of Campylobacter jejuni (GenBank AAK91727.1) or equivalent (e.g., (GenBank CAR04561.1), a Neu5Ac synthase (e.g., neuB of C. jejuni (GenBank AAK91726.1) or equivalent, (e.g., Flavobacterium limnosediminis sialic acid synthase, GenBank WP_023580510.1), and/or a CMP-Neu5Ac synthetase (e.g., neuA of C. jejuni (GenBank AAK91728.1) or equivalent, (e.g., Vibrio brasiliensis CMP-sialic acid synthase, GenBank WP_006881452.1). Disclosed as SEQ ID NO: 44 herein is an example of a neuBCA gene cluster from Campylobacter jejuni, alternative functional variants are also suitable for making a sialate sugar nucleotide in a genetically modified cell.

Furthermore, the genetically modified cell preferably has a deficient sialic acid catabolic pathway. By “sialic acid catabolic pathway” is meant a sequence of reactions, usually controlled, and catalysed by enzymes, which results in the degradation of sialic acid. An exemplary sialic acid catabolic pathway described hereafter is the E. coli pathway. In this pathway, sialic acid (Neu5Ac; N-acetylneuraminic acid) is degraded by the enzymes NanA (N-acetylneuraminic acid lyase) and NanK (N-acetylmannosamine kinase) and NanE (N-acetylmannosamine-6-phosphate epimerase), all encoded from the nanATEK-yhcH operon, and repressed by NanR (http://ecocyc.org/ECOLI). A inactivated sialic acid catabolic pathway is rendered in the E. coli host by introducing one or more mutations in the endogenous nanA (N-acetylneuraminate lyase) (e.g., GenBank Accession Number D00067.1(GL216588)) and/or nanK (N-acetylmannosamine kinase) genes (e.g., GenBank Accession Number (amino acid) BAE77265.1 (GL85676015)), and/or nanE (N-acetylmannosamine-6-phosphate epimerase, GI: 947745), incorporated herein by reference). Optionally, the nanT (N-acetylneuraminate transporter) gene is also inactivated or mutated. By inactivated is meant that the coding sequence has been altered such that the resulting gene product is functionally inactive or encodes for a gene product with less than 100%, e.g., 90%, 80%, 70%, 60%, 50%, 40%, 30% or 20% of the activity of the native, naturally occurring, endogenous gene product. Thus, in the present invention, nanA, nanK, nanE, and/or nanT genes are preferably inactivated.

### Lactose Importer

The genetically modified cell described herein is capable of importing the side-product produced as the leaving group in the transglycosylation process (enzymatic process) of the hybrid process into the cell.

In embodiments the genetically modified cell comprises a side-product importer. Preferably the side-product importer can import one or more of the following side-products lactose, 2′FL and/or 3FL.

Most lactose importers are capable of importing both lactose and 2′FL. In embodiments where the side-product is lactose or 2′FL the genetically modified cell has a functional lactose importer or a 2′FL importer. Lactose importers are well known in a wide variety of species including bacteria and yeasts.

The lactose importer can for example be a lactose permease. The lactose permease may be an endogenous lactose permease natively expressed by the cell used to produce the first oligosaccharide.

In one or more embodiment(s), the genetically engineered cell comprises one or more lactose permease genes which is/are overexpressed.

The genetically engineered cell may comprise least one, such as at least two, three, four, nucleic acid sequence(s) encoding a lactose permease.

In one or more further exemplary embodiment(s) the one or more lactose permease(s) is/are encoded by a heterologous and/or recombinant nucleic acid sequence. The native lactose permease may be genetically engineered to for example place it under control of a stronger promoter than the native promoter, thereby generating a recombinant lactose permease gene overexpressing the native lactose permease protein.

In one or more preferred exemplary embodiment(s) the nucleic acid sequence(s) encoding the one or more lactose permease(s) is a native gene to the genetically engineered cell.

In E. coli the lactose permease is encoded by the lacY gene in the lac operon. In exemplary embodiments, the lactose permease in the genetically modified cell is LacY from E. coli. Preferably the Lactose permease comprises or consists of an amino acid sequence of SEQ ID NO: 3 or a functional homologue thereof, such as a lactose permease having an amino acid sequence which is at least 80%, such as at least 90%, such as at least 95%, such as at least 99%, or 100% identical to SEQ ID NO: 3.

### Transporter Protein

The genetically engineered cell of the present disclosure comprises at least one nucleic acid sequence encoding one or more transporter protein(s) capable of exporting the first oligosaccharide from the cell into the culture medium. The genetically engineered cell of the present disclosure preferably expresses a heterologous Major Facilitator Superfamily (MFS) transporter protein.

The transporters of the Major Facilitator Superfamily (MFS) facilitate the transport of molecules, such as but not limited to oligosaccharides, across the cellular membranes.

The term “MFS transporter” in the present context means a protein that facilitates transport of an oligosaccharide, preferably an HMO, through or across a cell membrane, from the cell cytosol to the cell periplasm and/or medium. Preferably, the MFS transporter transports an HMO/oligosaccharide synthesized by the genetically modified cell as described herein. Additionally, or alternatively, the MFS transporter may also facilitate efflux of molecules that are not considered HMO or oligosaccharides, such as lactose, glucose, cell metabolites and/or toxins. In a preferred embodiment the MFS transporter is capable of exporting 2′FL, 3FL, 3′SL, 6′SL, LNT-II, LNT and/or LNnT from the cell cytosol to the cell medium.

In the context of the present invention the lactose permease is not considered to be a heterologous MFS transporter.

In one or more exemplary embodiment(s), the MFS transporter is selected from the group consisting of Bad, Nec, YberC, Fred, Vag and Marc.

The genetically modified cell of the present disclosure thus in one or more exemplary embodiment(s) expresses a heterologous MFS transporter protein selected from the group consisting of Vag, Nec, Fred, Marc, YberC, Bad and a functional homologue of any one of Vag, Nec, Fred, Marc, YberC or Bad having an amino acid sequence which is 80% identical to said.

### Bad

The MFS transporter protein identified herein as “Bad protein” or “Bad transporter” or “Bad”, has an amino acid sequence corresponding to the GenBank accession ID WP_017489914.1.

In one or more embodiment(s) of the invention, the genetically engineered cell expresses the heterologous MFS transporter protein bad or a functional homologue thereof, having an amino acid sequence which is at least 80%, such as at least 90%, such as at least 95%, such as at least 99%, or 100% identical to the GenBank accession ID WP_017489914.1

### Nec

The MFS transporter protein identified herein as “Nec protein” or “Nec transporter” or “Nec”, interchangeably, has an amino acid sequence corresponding to the GenBank accession ID WP_092672081.1.

In one or more embodiment(s) of the invention, the genetically engineered cell expresses the heterologous MFS transporter protein Nec or a functional homologue thereof, having an amino acid sequence which is at least 80%, such as at least 90%, such as at least 95%, such as at least 99%, such as 100% identical to the amino acid sequence of the GenBank accession ID WP_092672081.1.

Nec is in particularly suitable for transporting 2′FL, DFL, 3′SL, 6′SL and LNFP-I.

### YberC

The MFS transporter protein identified herein as “YberC protein” or “YberC transporter” or “YberC”, interchangeably, has an amino acid sequence corresponding the GenBank accession ID EEQ08298.1.

In one or more embodiment(s) of the invention, the genetically engineered cell expresses the heterologous MFS transporter protein YberC or a functional homologue thereof, having an amino acid sequence which is at least 80%, such as at least 90%, such as at least 95%, such as at least 99%, such as 100% identical to the amino acid sequence of the GenBank accession ID EEQ08298.1.

YberC is particularly useful in transporting LNT.

### Fred

The MFS transporter protein identified herein as “Fred protein” or “Fred transporter” or “Fred”, interchangeably, has an amino acid sequence corresponding the GenBank accession ID WP_087817556.1.

In one or more exemplary embodiment(s), the MFS transporter, expressed according to the present disclosure is Fred. The genetically engineered cell of the present disclosure thus in one or more exemplary embodiment(s) expresses a heterologous MFS transporter protein that is Fred.

In one or more embodiment(s) of the invention, the genetically engineered cell expresses the heterologous MFS transporter protein fred or a functional homologue thereof, having an amino acid sequence which is at least 80%, such as at least 90%, such as at least 95%, such as at least 99%, such as 100% identical to the amino acid sequence of the GenBank accession ID WP_087817556.1.

Fred is particularly useful in transporting 3′SL and 6′SL.

### Vag

The MFS transporter protein identified herein as “Vag protein” or “Vag transporter” or “Vag”, interchangeably, has an amino acid sequence corresponding to SEQ ID NO: 51 or the GenBank accession ID WP_048785139.1.

In one or more embodiment(s) of the invention, the genetically engineered cell expresses the heterologous MFS transporter protein vag or a functional homologue thereof, having an amino acid sequence which is at least 80%, such as at least 90%, such as at least 95%, such as at least 99%, such as 100% identical to the amino acid sequence of SEQ ID NO: 51 or to the amino acid sequence of the GenBank accession ID WP_048785139.1.

Vag is particularly useful in transporting LNnT.

### Marc

The MFS transporter protein identified herein as “Marc protein” or “Marc transporter” or “Marc”, interchangeably, has an amino acid sequence corresponding to SEQ ID NO: 2 or the GenBank accession WP_060448169.1.

In one or more embodiment(s) of the invention, the genetically engineered cell expresses the heterologous MFS transporter protein marc or a functional homologue thereof, having an amino acid sequence which is at least 80%, such as at least 90%, such as at least 95%, such as at least 99%, such as 100% identical to the amino acid sequence of SEQ ID NO: 2 or the GenBank accession WP_060448169.1.

Marc is particularly useful in transporting 3FL.

The genetically engineered cell of the present disclosure thus in one or more exemplary embodiment(s) expresses a heterologous MFS transporter protein which is either Vag, Nec, Fred, Marc, YberC or Bad.

In one or more exemplary embodiment(s), the genetically engineered cell of the present disclosure expresses a functional homologue of Vag, Nec, Fred, Marc, YberC and/or Bad having an amino acid sequence which is at least 70%, 80%, 85%, 90%, 95% or at least 99% identical to the Vag, Nec, Fred, Marc, YberC and/or Bad GenBank ascension numbers indicated above.

In a presently preferred embodiment, the MFS transporter expressed is Nec.

In an especially preferred embodiment, the MFS transporter expressed is YberC.

In an especially preferred embodiment, the MFS transporter expressed is Marc.

In an especially preferred embodiment, the MFS transporter expressed is Vag.

In an especially preferred embodiment, the MFS transporter expressed is Fred

### Sequence Identity

The term “sequence identity” as used herein describes the relatedness between two amino acid sequences or between two nucleotide sequences, i.e., a candidate sequence (e.g., a sequence of the invention) and a reference sequence (such as a prior art sequence) based on their pairwise alignment. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labelled “identity” (obtained using the -nobrief option) is used as the percent identity. Generally sequence identity may be calculated as follows: (Identical Residues×100)/(Length of Aligned region).

For purposes of the present invention, the sequence identity between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labelled “identity” (obtained using the -nobrief option) is used as the percent identity. Generally sequence identity may be calculated as follows: (Identical Deoxyribonucleotides×100)/(Length of Aligned region).

### Functional Homologue

A functional homologue or functional variant of a protein/nucleic acid sequence as described herein is a protein/nucleic acid sequence with alterations in the genetic code or amino acid sequence, which retains its original functionality. A functional homologue may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence. In embodiments of the present invention, the functional homologue is at least 80% identical, such as at least 85% identical such as at least 90% identical, such as such as at least 95% identical to the protein/nucleic acid sequence indicated in connection with a give protein, nucleic acid or gene.

Functional variants of proteins or peptides may contain conservative amino acid substitution(s) compared to their native, i.e., non-mutated physiological, sequence. Those amino acid sequences as well as their encoding nucleotide sequences in particular fall under the term functional variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g., side chains which have a hydroxyl function. This means that e.g., an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Truncations, insertions and substitutions are possible, in particular, at those sequence positions which cause no modification to the three-dimensional structure or do not affect the binding region. Modifications to a three-dimensional structure by insertion(s) or deletion(s) can easily be determined e.g., using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

Furthermore, functional variants of proteins or peptides as defined herein may also comprise those sequences, wherein nucleotides of the nucleic acid are exchanged according to the degeneration of the genetic code, without leading to an alteration of the respective amino acid sequence of the protein or peptide, i.e., the amino acid sequence or at least part thereof may not differ from the original sequence in one or more mutation(s) within the above meaning.

### Retrieving/Harvesting

The sialylated and/or a fucosylated oligosaccharide of at least four monosaccharide produced by the hybrid method described herein can be retrieved from the culture medium of the process. In the present context, the term “retrieving” is used interchangeably with the term “harvesting”. Both “retrieving” and “harvesting” in the context relate to collecting the produced HMO(s) from the culture/broth following the termination of the process. Preferably, the biomass is discarded since this only contain the first oligosaccharide.

The transglycosidase enzyme is preferably deactivated prior to the harvest of the cells. Non-limiting methods suitable for deactivating the transglycosidase can be selected from i) heating the fermentation broth to a temperature that denatures the enzyme, ii) adding a protease to the culture broth at the end of fermentation to hydrolyze the enzyme or iii) change the pH of the culture such that it is outside the activity range of the enzyme.

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions.

After recovery of the hybrid process medium, the HMO mixture/composition is available for further processing and purification. It may be desirable to isolate individual HMOs from the HMO mixture to obtain e.g., a purified or enriched sialylated and/or a fucosylated oligosaccharide of at least four monosaccharide. Alternatively, the HMO mixture produced by the hybrid process can be purified to remove lactose and other metabolic non-HMO byproducts (e.g., by ultra-filtration and/or nanofiltration) and the HMO mixture or HMO composition can be used as it is.

The purification of the HMO composition or a specific component of the HMO mixture can be done according to procedures known by the skilled artesian. For example, HMOs can be purified according to the procedures known in the art, e.g., such as described in in WO2015/188834, WO2017/182965 or WO2017/152918, wherein the latter describes purification of HMOs.

### Mixtures of Oligosaccharides, Such as HMOs, and Compositions Thereof

Complex fucosylated and/or sialylated HMOs, such as FSL, LNFP-III, LNDFH-I, LST-a and LST-c and mixtures comprising FSL, LNFP-III, LNDFH-I, LST-a or LST-c are highly relevant as either a nutritional supplement or as a therapeutic as described in the section of uses of HMO mixtures described in the present application.

An aspect of the present disclosure is a mixture or composition of HMOs consisting essentially of:
a) at least 50 wt % FSL, below 45 wt % 3FL, and below 2 wt % 3′SL and below 3 wt % lactose, or
b) at least 60 wt % LNDFH-I, below 5 wt % LNFP-I, below 35 wt % 3FL and below 5 wt % lactose, or
c) at least 70 wt % LNDFH-I, below 10 wt % LNFP-I, below 25 wt % 3FL and below 3 wt % lactose, or
d) at least 40 wt % LST-c, below 25 wt % LNnT, below 25 wt % 6′SL and below 10 wt % lactose, or
e) at least 55 wt % LST-a, below 40 wt % LNT, below 15 wt % 3′SL and below 2 wt % lactose, and
   wherein the total composition constitutes 100 wt % of the components and the composition is a mixture of at least 2 components.

In embodiments the composition or mixture of HMOs consists essentially of
a) at least 50 wt % FSL, between 20 to 45 wt % 3FL, and between 0.1 to 2 wt % 3′SL and between 0 to 3 wt % lactose, or
b) at least 60 wt % LNDFH-I, between 0.1 to 8 wt % LNFP-I, between 15 to 35 wt % 3FL and between 0 to 5 wt % lactose, or
c) at least 70 wt % LNDFH-I, between 0.1 to 10 wt % LNFP-I, between 5 to 20 wt % 3FL and between 0 to 3 wt % lactose, or
d) at least 50 wt % LST-c, between 15 to 25 wt % LNnT, between 15 to 25 wt % 6′SL and between 0 to 7 wt % lactose,
e) at least 50 wt % LST-a, between 15 to 40 wt % LNT, between 0 to 15 wt % 3′SL and between 0 to 2 wt % lactose and
   wherein the total composition constitutes 100 wt % of the components.

In other embodiments, the mixture of HMOs described herein consists essentially of 40-50 wt % 3FL, and 50-60 wt % FSL, wherein the total composition constitutes 100 wt % of the components.

In other embodiments, the mixture of HMOs described herein consists essentially of 45 wt % 3FL, and 55 wt % FSL.

In other embodiments, the mixture of HMOs described herein consists essentially of 45-55 wt % LST-c, and 20-30 wt % LNnT and 20-30 wt % 6′SL, wherein the total composition constitutes 100 wt % of the components.

In other embodiments, the mixture of HMOs described herein consists essentially of 50 wt % LST-c, and 25 wt % LNnT and 25 wt % 6′SL.

In other embodiments, the mixture of HMOs described herein consists essentially of 60 to 80 wt % LST-a and 20 to 30 wt % LNT and 5 to 15 wt % 3′SL, wherein the total composition constitutes 100 wt % of the components.

In other embodiments, the mixture of HMOs described herein consists essentially of 65 wt % LST-a and 25 wt % LNT and 10 wt % 3′SL.

In other embodiments, the mixture of HMOs described herein consists essentially of 50 to 60 wt % LST-a, and 40 to 50 wt % LNT, wherein the total composition constitutes 100 wt % of the components.

In other embodiments, the mixture of HMOs described herein consists essentially of 55 wt % LST-a and 45 wt % LNT.

As shown in the examples, the hybrid method of the present disclosure allows for improved ratios of the desired complex fucosylated or sialylated HMO over the donor and/or acceptor HMO as compared to the conventional in vitro process where the ratios between the individual HMOs is limited by the kinetic barrier of the enzymatic reaction, preventing shifts of the equilibrium between the components of the in vitro enzymatic reaction.

In embodiments the composition and/or mixture of HMOs has a molar ratio of FSL:3′SL above 100:1 and the molar ratio of FSL:3FL is above 1:1.

In other embodiments the composition and/or mixture of HMOs has a molar ratio of LNDFH-I:LNFP-I above 50:1 and the molar ratio of LNDFH-I:3FL is in the range of 1.5:1 to 2.6:1.

In other embodiments the composition and/or mixture of HMOs has a molar ratio of LST-c:LNnT above 2.5:1 and the molar ratio of LST-c:6′SL above 2.5:1.

In other embodiments the composition and/or mixture of HMOs has a molar ratio of LST-a:LNT above 1.5:1 and the molar ratio of LST-a:3′SL above 8:1.

In embodiments, the composition comprising a mixture of HMOs is a nutritional composition. Nutritional compositions are for example infant formula or medical nutritional compositions.

In embodiments, the composition comprising a mixture of HMOs is dietary supplement.

In embodiments, the composition comprising a mixture of HMOs is a pharmaceutical composition.

### Use of HMO Mixtures and Compositions

Clinical data in infants, indicate that human milk oligosaccharide supplements may help to develop the desired microbiota by serving as a food source for the good bacteria in the intestine. Naturally occurring in breast milk, HMOs have evolved over thousands of years, with HMO research (clinical and preclinical) now suggesting that specific HMO's at the correct level of supplementation can provide us with unique health benefits. In particular, Human Milk Oligosaccharide supplements may help support immunity and gut health including a support a balanced microbiome, with a potential role in cognitive development, which may open future innovation opportunities.

Accordingly, in embodiments, the invention relates to the use of a mixture or composition disclosed herein in infant nutrition.

The present invention also relates to the use of a mixture or composition disclosed herein as a dietary supplement or medical nutrition or a pharmaceutical composition.

The mixtures or composition of HMOs produced according to the method described herein may be used to enhance the beneficial bacteria in the gut microbiome. Beneficial bacteria are for example bacteria of the Bifidobacterium sp., Lactobacillus sp. or Barnesiella sp. The enhancement of beneficial bacteria may in turn lead to increased production of short chain fatty acids (SCFAs) such as acetate, propionate and butyrate which have been shown to have many benefits in infants and young children, such as inhibition of pathogen bacteria, prevention of infection and diarrhoea, reduced risk of allergy and metabolic disorders (see for example WO2006/130205, WO 2017/129644, WO2017/129649).

The mixtures or composition of HMOs produced according to the method described herein may be used to reduce the abundance of undesirable viruses and bacteria in the gut microbiome. Examples of pathogenic bacteria and viruses that may be reduced by the HMO mixtures described herein are including Candida albicans, Clostridium difficile, Enterococcus faecium, Escherichia coli, Helicobacter pylori, Streptococcus agalactiae, Shigella dysenteriae, Staphylococcus aureus, nora virus and rota virus. Each composition described herein can also be used to treat and/or reduce the risk of a broad range of bacterial infections of a human.

The mixtures or composition of HMOs produced according to the method described herein may be used to increase the regeneration and viability of lyophilized probiotics, including probiotics of Bifidobacterium sp, Lactobacillus sp., in particular increased regeneration and/or viability and/or shelf-life in an acidic environment, such as the stomach or acidic food products, is an advantage using the HMO mixtures described herein. Examples of Bifidobacterium sp which may have increased regeneration and viability are Bifidobacterium animals lactis BB12 DSM 32269, Bifidobacterium animals lactis BIF6, Bifidobacterium longum DSM 32946, Bifidobacterium longum BB536, Bifidobacterium bifidum DSMZ 32403, Bifidobacterium infantis, Bifidobacterium breve DSM 33789, Bifidobacterium infantis SP37 DSM 32687, Bifidobacterium adolescentis DSM 34065 and/or Bifidobacterium animalis ssp. animalis DSM 16284. Examples of Lactobacillus sp which may have increased regeneration and viability are Lactobacillus rhamnosus GG DSM 32550, Lactobacillus rhamnosus 19070-2 DSM 26357, Lactobacillus rhamnosus GG, Lactobacillus rhamnosus LBrGG, Lactobacillus reuteri DSM 12246, Lactobacillus plantarum TIFN101, Lactobacillus gasseri Lg-36 200B FloraFit Danisco, Lactobacillus casei DSM 32382, Lactobacillus paracasei, Lactobacillus plantarum PS 128, Lactobacillus plantarum (Sacco) DSM 32383, Lactococcus lactis PAREVE, Lactobacillus paracasei ssp. Paracasei and/or Lactobacillus Probio-Tec® LGG®, Limosilactobacillus reuteri S12 DSM 33752.

In the context of the present application “Regeneration” means the process of regaining/restoring a dried bacteria's viability (i.e., “reviving” the bacterial cells by rehydration, wherein “rehydration” means restoring fluid). This process is also sometimes referred to as “reconstitution”.

In the context of the present application “Viability” is the ability of a bacterial cell to live and function as a living cell. One way of determining the viability of bacterial cells is by spreading them on an agar plate with suitable growth medium and counting the number of colonies formed after incubation for a predefined time (plate counting). Alternatively, FACS analysis may be used.

In the context of the present application “Improving the regeneration” of Bifidobacterium sp and/or Lactobacillus sp bacteria means to increase the amount (number) of Bifidobacterium sp and/or Lactobacillus sp. bacteria successfully regenerating/reviving compared to the respective control (i.e., the amount/number of Bifidobacterium sp and/or Lactobacillus sp. bacteria without the addition of HMO).

In the context of the present application “Improving the viability” of Bifidobacterium sp and/or Lactobacillus sp bacteria means to increase the amount (number) of viable Bifidobacterium sp and/or Lactobacillus sp. bacteria compared to the respective control (i.e., the amount/number of Bifidobacterium sp and/or Lactobacillus sp. bacteria without the addition of HMO).

In the context of the present application “acidic” means having a pH below 7.0 (for example, having a pH≤6.0, or ≤5.0, or ≤4.0, or ≤3.0, or in the range of 1.0-6.0, such as from 2.0 to 5.0). The pH measured in the stomach is in the range of about 1.5-3.5. The pH measured in a healthy vagina is in the range of about 3.8-5.0. The pH of fruit juices is in the range of about 2.0-4.5.

The mixtures or composition of HMOs produced according to the method described herein, may be used to extend the shelf life of probiotics, such as Bifidobacterium sp, and/or Lactobacillus sp.

An embodiment of the present invention is a composition comprising a mixture of HMOs as described herein, in particular in the section “Mixtures of HMOs”, and one or more probiotics. Preferably, the probiotic is a Bifidobacterium sp and/or Lactobacillus sp such as any of the specific species mentioned above.

The mixtures or composition of HMOs produced according to the method described herein, may be used to improve the flowability of a powder or decrease the viscosity of a liquid.

The mixtures or composition of HMOs produced according to the method described herein are used in a nutritional composition. Nutritional compositions are for example, an infant formula, a rehydration solution, or a dietary maintenance, medical nutrition or supplement for elderly individuals or immunocompromised individuals. Macronutrients such as edible fats, carbohydrates and proteins can also be included in such anti-infective compositions. Edible fats include, for example, coconut oil, soy oil and monoglycerides and diglycerides. Carbohydrates include, for example, glucose, edible lactose and hydrolysed cornstarch. Proteins include, for example, soy protein, whey, and skim milk. Vitamins and minerals (e. g. calcium, phosphorus, potassium, sodium, chloride, magnesium, manganese, iron, copper, zinc, selenium, iodine, and Vitamins A, E, D, C, and B complex) can also be included in such anti-infective compositions.

### Embodiments

The following embodiments of the present invention may be used in combination with any other embodiments described herein.

1. A method for producing a oligosaccharide from a donor oligosaccharide and an acceptor oligosaccharide or disaccharide, said method comprising the steps of:
a) cultivating a first genetically modified cell capable of producing a first oligosaccharide in a culture medium supplied with a carbon source, wherein the genetically modified cell comprises one or more nucleic acids encoding:
   i) at least one recombinant glycosyltransferase, and
   ii) at least one pathway to produce a nucleotide-activated sugar, and
b) supplying a second disaccharide or oligosaccharide to the culture medium, and
c) making an enzyme with transglycosidase activity available in the culture medium,
d) incubating the first oligosaccharide, the second oligosaccharide and the transglycosidase enzyme in the culture medium to form a third complex oligosaccharide of at least four monosaccharide units.

2. A method for producing a sialylated and/or a fucosylated oligosaccharide from a donor oligosaccharide and an acceptor oligosaccharide, said method comprising the steps of:
a) cultivating a first genetically modified cell capable of producing a first oligosaccharide in a culture medium supplied with a carbon source, wherein the genetically modified cell comprises one or more nucleic acids encoding:
   i) at least one recombinant glycosyltransferase, and
   ii) at least one pathway to produce a nucleotide-activated sugar, and
b) supplying a second disaccharide or oligosaccharide to the culture medium, wherein either the first or the second oligosaccharide is a fucosyl- or sialyl-donor or lacto-N-biose-donor oligosaccharide; and
c) making an enzyme with transglycosidase activity available in the culture medium,
d) incubating the first oligosaccharide, the second oligosaccharide and the transglycosidase enzyme in the culture medium to form a third sialylated and/or fucosylated oligosaccharide of at least four monosaccharide units.

3. The method according to item 1 or 2, wherein either the first or the second oligosaccharide is a fucosylated or sialylated donor oligosaccharide and the other disaccharide or oligosaccharide is an acceptor oligosaccharide.

4. The method according to item 1 or 3, wherein if the second disaccharide or oligosaccharide is a disaccharide this is not lactose.

5. The method according to item 1 or 4, wherein if the second disaccharide or oligosaccharide is a disaccharide this is lacto-N-biose (LNB) or N-acetyllactosamine (LacNAc).

6. The method according to any one of the preceding items, wherein the oligosaccharide is a complex oligosaccharide of at least four monosaccharide units or an oligosaccharide of at least 3 monosaccharide units with a lacto-N-biose (LNB) moiety or N-acetyllactosamine (LacNAc) moiety at the reducing end.

7. The method according to item 6, wherein the oligosaccharide of at least 3 monosaccharide units is Lewis X or Lewis A.

8. The method according to item 1 or 7, wherein the second disaccharide or oligosaccharide is supplied to the culture medium by adding it.

9. The method according to item 1 or 7, wherein the first genetically modified cell is capable of producing both the first and the second oligosaccharide.

10. The method according to item 1 to 7, wherein the second oligosaccharide is supplied to the culture medium from a second genetically modified cell capable of producing the second oligosaccharide.

11. The method according to item 10, wherein the second genetically modified cell is co-cultured with the first genetically modified cell of item 1 a).

12. The method according to item 10 or 11, wherein the second genetically modified cell
a) comprises at least one recombinant nucleic acid sequence encoding at least one glycosyltransferase, and
b) comprises at least one a biosynthetic pathway to produce a nucleotide-activated sugar,
c) is capable of growing on a second carbon source while showing limited or no growth on the carbon source (first carbon source) which the genetically modified cell in step a) of item 1 (the first genetically modified cell) is capable of growing on.

13. The method according to item 11 or 12, wherein the first genetically modified cell of item 1 a) has limited or no growth on the second carbon source.

14. The method according to item 11 to 13, wherein one of the genetically modified microbial cells is capable of growing on sucrose and comprises one or more nucleic acid sequences encoding a PTS-dependent sucrose utilization system or a nucleic acid encoding a sucrose invertase or sucrose hydrolase enabling the assimilation of sucrose by said cell.

15. The method according to item 14, wherein the PTS-dependent sucrose utilization system is encoded by scrY (SEQ ID NO: 47), scrA (SEQ ID NO: 48), scrB (SEQ ID NO: 49) and optionally scrR (SEQ ID NO: 50) or by the cscABKR-gene cluster (SEQ ID NO: 52)

16. The method according to item 14 or 15, wherein said cell has reduced or no ability to grow on one or more carbon sources selected from the group consisting of glucose, glycerol, galactose, maltose, sorbitol, arabinose and fructose.

17. The method according to item 11 to 13, wherein one of the genetically modified microbial cells is capable of growing on glucose and comprises one or more nucleic acids encoding one or more glucose transport systems.

18. The method according to item 17, wherein the glucose transport system is a PTS-dependent glucose transport system selected from the group consisting of:
i) glucose PTS complex components IICB^{Glc};
ii) beta-glucoside PTS complex components—IIABC^{Bgl};
iii) mannose PTS complex components—IICD^{Man};
iv) N-acetylglucosamine PTS complex components—IIABCN^{Nag}; and
v) maltose/maltodextrin PTS complex—IICB^{malX}

19. The method according to item 17, wherein the glucose transport system is selected from the group consisting of:
i) galactose:H+ symporter GalP;
ii) glucose uptake protein GlcU;
iii) sodium/glucose transporter family (SGLT);
iv) galactose/glucose ABC transporter (mglABC) system;
v) trehalose/maltose/sucrose/palatinose (TMSP)—ABC transporter (malEFG) system;
vi) glucose/mannose ABC transporter (glcEFG) system;
vii) glucose proton symporter (glcP);
viii) glucose facilitator (glf); and
ix) hexose transporters (HXT).

20. The method according to item 17 to 19, wherein said cell has reduced or no ability to grow on one or more carbon sources selected from the group consisting sucrose, glycerol, galactose, maltose, sorbitol, arabinose and fructose.

21. The method according to item 11 to 13, wherein the genetically modified microbial cell capable of growing on glycerol comprises one or more nucleic acids encoding one or more glycerol transport systems.

22. The method according to item 21, wherein the glycerol transport system is selected from glycerol facilitator or glycerol/H+ symporter.

23. The method according to item 21 or 22, wherein said cell has reduced or no ability to grow on one or more carbon sources selected from the group consisting of sucrose, glucose, galactose, maltose, sorbitol, arabinose and fructose.

24. The method according to item 11 to 23, wherein the first genetically modified microbial cell grows on glucose or glycerol and the second genetically modified microbial cell grow on sucrose.

25. The method according to item 11 to 13, wherein the genetically modified microbial cell capable of growing on galactose comprises one or more nucleic acids encoding one or more galactose transport systems.

26. The method according to item 25, wherein the galactose transport system is selected from galactose:H+ symporter, the galactose/glucose ABC transporter (mglABC) system, the PTSLac (lacFE) system and/or the sodium/glucose transporter family (sglT).

27. The method according to item 25 or 26, wherein said cell has reduced or no ability to grow on one or more carbon sources selected from the group consisting of sucrose, glucose, glycerol, maltose, sorbitol, arabinose and fructose.

28. The method according to item 11 to 13, wherein the genetically modified microbial cell capable of growing on fructose comprises one or more nucleic acids encoding one or more fructose transport systems.

29. The method according to item 28, wherein the galactose transport system is selected from PTS complex components IIABC^{Fru}, glucose PTS complex components IICB^{Glc} and the fructose transporter FruP.

30. The method according to item 28 or 29, wherein said cell has reduced or no ability to grow on one or more carbon sources selected from the group consisting of sucrose, glucose, glycerol, maltose, sorbitol, arabinose and galactose.

31. The method according to item 11 to 13, wherein the genetically modified microbial cell capable of growing on maltose comprises one or more nucleic acids encoding one or more maltose transport systems.

32. The method according to item 31, wherein the galactose transport system is selected from MalFGK ABC superfamily transport system and/or maltose/maltodextrin PTS complex.

33. The method according to item 31 or 32, wherein said cell has reduced or no ability to grow on one or more carbon sources selected from the group consisting of sucrose, glucose, glycerol, fructose, sorbitol, arabinose and galactose.

34. The method according to item 11 to 13, wherein the genetically modified microbial cell capable of growing on arabinose comprises one or more nucleic acids encoding one or more arabinose transport systems.

35. The method according to item 34, wherein the arabinose transport system is selected from AraFGH ABC superfamily transport system and/or arabinose-proton symporter AraE.

36. The method according to item 34 or 35, wherein said cell has reduced or no ability to grow on one or more carbon sources selected from the group consisting of sucrose, glucose, glycerol, fructose, sorbitol, and galactose.

37. The method according to item 11 to 13, wherein the genetically modified microbial cell capable of growing on sorbito comprises one or more nucleic acids encoding one or more sorbitol transport systems.

38. The method according to item 37, wherein the sorbitol transport system is selected from the sorbitol-PTS system (EIIBC^{srl}).

39. The method according to item 37 or 38, wherein said cell has reduced or no ability to grow on one or more carbon sources selected from the group consisting of sucrose, glucose, glycerol, fructose, arabinose and galactose.

40. The method according to item 1 to 39, wherein said genetically modified cell(s) exports into the culture medium said oligosaccharide produced by said cell(s).

41. The method according to item 1 to 40, wherein the first genetically modified cell is capable of producing a first oligosaccharide of at least three monosaccharide units into a culture medium.

42. The method according to item 8 to 41, wherein the second genetically modified cell is capable of producing a second oligosaccharide or disaccharide in situ in the culture medium in which the first genetically modified cell is cultured.

43. The method according to item 42, wherein the second genetically modified cell produced a second oligosaccharide of at least three monosaccharide units.

44. The method according to item 42, wherein the second genetically modified cell produced a disaccharide selected from lacto-N-biose (LNB) or N-acetyllactosamine (LacNAc).

45. The method according to any one of items 1 to 44, wherein at least one of the genetically modified cells further comprises a nucleic acid encoding a side-product importer.

46. The method according to item 45, wherein the side-product importer is a lactose importer.

47. The method according to any one of items 1 to 43 or 45 or 46, wherein the first and/or second oligosaccharide is produced from lactose as the initial substrate.

48. The method according to any one of items 1 to 47, wherein the donor oligosaccharide is selected from the group consisting of a fucosylated oligosaccharide of three to five monosaccharide units, a sialylated oligosaccharide of three to five monosaccharide units and a neutral core oligosaccharide three to four monosaccharide units.

49. The method according to any one of items 1 to 48, wherein the donor oligosaccharide is a fucosyl- or sialyl-donor oligosaccharide selected from a fucosyllactose or a sialyllactose or a sialyl-N-acetyllactosamine or a sialyl-lacto-N-biose and the other oligosaccharide is an acceptor oligosaccharide.

50. The method according to item 1 to 45, wherein the first genetically modified cell is capable of importing 2′FL, or 3FL.

51. The method according to item 50, wherein the first genetically modified cell is capable of producing a first oligosaccharide of four monosaccharide units.

52. The method according to item 50 or 51, wherein the first oligosaccharide is selected from DFL or FSL.

53. The method according to item 1 to 45, wherein the first genetically modified cell is capable of importing N-acetyllactosamine or lacto-N-biose.

54. The method according to any one of the preceding items, wherein the transglycosidase enzyme is selected from the group consisting of α-1,2-tranfucosidase, α-1,3-transfucosidase, α-1,3/4-transfucosidase, α-2,3-transsialidase, α-2,6-transsialidase, β-N-acetylglucosaminidase, trans-lacto-N-biosidase and trans-β-galactosidase.

55. The method according to item 54, wherein the transglycosidase enzyme is selected from the transsialidases in table 2 or a functional homologue thereof having an amino acid sequence of at least 80% compared to an individual transsialidase sequence in table 2.

56. The method according to item 54 or 55, wherein the transsialidase is an enzyme selected from SEQ ID NO: 13, 14, 40, 41 or 60.

57. The method according to any one of the preceding items, wherein the transglycosidase enzyme is selected from the transfucosidases in table 3 or a functional homologue thereof having an amino acid sequence of at least 80% compared to an individual transsialidase sequence in table 3.

58. The method according to item 54 or 57, wherein the transfucosidase is an enzyme selected from SEQ ID NO: 19, 30, 39 or 59.

59. The method according to any one of the preceding items, wherein the enzyme with transglycosidase activity is
i) a transfucosidase if the donor oligosaccharide is a fucosyl oligosaccharide, or
ii) a transsialidase if the donor oligosaccharide is a sialyl oligosaccharide or
iii) a trans-lacto-N-biosidase.

60. The method according to any one of the preceding items, wherein the transglycosidase enzyme is either added to the culture medium during the cultivation or is expressed from a recombinant nucleic acid in said genetically modified cell.

61. The method according to item 60, wherein the transglycosidase enzyme is expressed from a recombinant nucleic acid in said genetically modified cell and exported to the culture medium from said cell.

62. The method according to any one of the preceding items, wherein the incubation in step d) of item 1 or 2 allow for transglycosylation of the acceptor oligosaccharide with the fucosyl-moiety or sialyl-moiety or lacto-N-biose-moiety of the donor oligosaccharide mediated by the transglycosidase activity provided in step c) of item 1 to form a third sialylated and/or fucosylated oligosaccharide of at least four monosaccharide units.

63. The method according to any one of items 54 to 62, wherein the hydrolytic activity of the transfucosidase enzyme results in below 5% fucose of the total molar % of the donor oligosaccharide and the third oligosaccharide in the culture at the end of process.

64. The method according to any one of items 54 to 62, wherein the hydrolytic activity of the transsialidase enzyme results in below 5% sialic acid of the total molar % of the donor oligosaccharide and the third oligosaccharide in the culture at the end of process.

65. The method according to any one of the preceding items, wherein the genetically modified cell(s) is cultivated in the presence of one or more carbon source selected from the group consisting of glucose, sucrose, fructose, arabinose, sorbitol, xylose, galactose, maltose and glycerol.

66. The method according to any one of the preceding items, wherein the cultivation in step a) is initiated in the presence of sufficient initial substrate for the cell to produce the first oligosaccharide.

67. The method according to item 66, wherein no additional initial substrate is added to the culture medium after the initiation of the cultivation.

68. The method according to item 1 to 66, wherein the initial substrate for the cell to produce the first oligosaccharide is added to the culture medium when the initial carbon source is consumed.

69. The method according to any one of items 60 to 68, wherein the transglycosidase is added to the culture medium and no additional initial substrate is added to the culture medium after addition of the transglycosidase.

70. The method according to any one of items 67 to 69, wherein the transglycosidase is added to the culture at a time point when the genetically modified cell has converted at least 50% of the initial substrate into the first oligosaccharide.

71. The method according to items 67 to 70, wherein the transglycosidase is added to the culture at a time point when less than 10% of the initial substrate initially added to the culture is left.

72. The method according to items 15 to 71, wherein the initial substrate for producing the first oligosaccharide is selected from lactose, N-acetyllactosamine, lacto-N-biose, 2′FL, 3FL or LNT-II.

73. The method according to items 15 to 72, wherein the initial substrate for producing the first and/or second oligosaccharide is lactose.

74. The method according to items 15 to 72, wherein the initial substrate for producing the first oligosaccharide is 2′FL.

75. The method according to items 15 to 72, wherein the initial substrate for producing the first oligosaccharide is N-acetyllactosamine (LacNAc).

76. The method according to items 15 to 72, wherein the initial substrate for producing the first oligosaccharide is lacto-N-biose (LNB).

77. The method according to items 15 to 72, wherein the initial substrate for producing the first oligosaccharide is LNT-II.

78. The method according to any one of items 1 to 8 or 40 to 42 or 45 to 74, wherein the second oligosaccharide is added to the culture medium at a rate corresponding to the rate of formation of the first oligosaccharide.

79. The method according to any one of items 1 to 8 Error! Reference source not found. or 40 to 42 or 45 to 77, wherein the second oligosaccharide is added to the culture medium at a faster rate than the rate of formation of the first oligosaccharide.

80. The method according to any one of items 1 to 8 or 40 to 42 or 45 to 77, wherein the second oligosaccharide or disaccharide is added to the culture medium in a molar amount of at least 2:1, such as at least 3:1, of the initial substrate added for producing the first oligosaccharide.

81. The method according to any one of the preceding items, wherein the cultivation is a fed-batch or continuous fed-batch fermentation, where after the initial carbon source(s) is consumed the carbon source(s) is fed to the culture medium throughout the fermentation.

82. The method according to item 81, wherein the carbon source(s) is fed at a rate that allows continues growth of the genetically modified cell(s).

83. The method according to item 81, wherein the carbon source(s) is fed at a rate where the culture(s) grows under carbon limitation.

84. The method according to any one of items 47 to 83, wherein the side-product importer is a lactose permease.

85. The method according to item 84, wherein the genetically modified cell expresses one or more recombinant lactose permease(s).

86. The method according to item 84 or 85, where the lactose permease is LacY comprising or consisting of SEQ ID NO: 3 or a functional variant thereof.

87. The method according to any one of items 1 to 86, wherein the third oligosaccharide is a complex oligosaccharide of at least four monosaccharide units selected from the group consisting of DFL, FSL, Lewis B, Lewis Y, sialyl-Lewis A, sialyl-Lewis X, LNFP-I, LNFP-II, LNFP-III, LNFP-IV, LNFP-V, LNFP-VI, LST-a, LST-b, LST-c, DSLNT, LNDFH-I, LNDFH-II, LNDFH-III, FLST-a, FLST-b, FLST-c, pLNH, pLNnH, LNH, LNnH, FLNH-I, FLNH-II, FLNH-III, FpLNH-I, FpLNnH II, DF-LNF-I, DF-LNF-II, DF-LNF-III, DF-para-LNH, DF-para LNnH, FLNnHa, FLNnHb, DFLNnH, TF-LNH, SLNH, FSLNH, SLNnH-I, FSLNnH-I, SLNnH-II, and DS-FLNH-II.

88. The method according to any one of items 1 to 87, wherein at least one of the first and second oligosaccharides is an HMO.

89. The method according to any one of items 1 to 88, wherein the first, second and third oligosaccharides are HMOs.

90. The method according to any one of the preceding items, wherein the third oligosaccharide is an HMO selected from the group consisting of DFL, FSL, LNFP-I, LNFP-II, LNFP-III, LNFP-V, LNFP-VI, LST-a, LST-b, LST-c, DSLNT, LNDFH-I, LNDFH-II, LNDFH-III, FLST-a, FLST-b, FLST-c, pLNH, pLNnH, LNH, LNnH, FLNH-I, FLNH-II, FLNH-III, FpLNH-I, FpLNnH-II, DF-LNF-I, DF-LNF-II, DF-LNF-III, DF-para-LNH, DF-para LNnH, FLNnHa, FLNnHb, DFLNnH, TF-LNH, SLNH, FSLNH, SLNnH-I, FSLNnH-I, SLNnH-II, and DS-FLNH-II.

91. The method according to any one of the proceeding items, wherein the first genetically modified cell produces a donor oligosaccharide selected from the group consisting of 2′FL, 3FL, DFL, LNFP-I, FSL, 3′SL, 6′SL, 3′SLacNAc, 3′SLNB, LST-a, LNT-II, LNT and LNnT.

92. The method according to any one of items 1 to 51 or 54 to 73 or 78 to 91, wherein the complex oligosaccharide is FSL and the method comprises the steps of:
a) cultivating a first genetically modified cell producing 3FL in a culture medium; and
b) supplying 3′SL to the culture medium of a); and
c) making an enzyme with transsialidase activity available in the culture medium; and
d) incubating the 3FL, 3′SL and the transsialidase enzyme in the culture medium to form FSL and lactose; and
wherein the lactose is recycled by the cell to produce more 3FL.

93. The method according to any one of items 1 to 51 or 54 to 73 or 78 to 91, wherein the complex oligosaccharide is LNDFH-I and the method comprises the steps of:
a) cultivating a first genetically modified cell producing 3FL in a culture medium; and
b) supplying LNFP-I to the culture medium of a); and
c) making an enzyme with transfucosidase activity available in the culture medium; and
d) incubating the 3FL, LNFP-I and the transfucosidase enzyme in the culture medium to form LNDFH-I and lactose; and
wherein the lactose is recycled by the cell to produce more 3FL.

94. The method according to any one of items 1 to 51 or 54 to 73 or 78 to 91, wherein the complex oligosaccharide is LNFP-III comprising the steps of:
a) cultivating a first genetically modified cell producing 3FL in a culture medium, and
b) supplying LNnT to the culture medium of a), and
c) making an enzyme with transfucosidase activity available in the culture medium, and
d) incubating the 3FL, LNnT and the transfucosidase enzyme in the culture medium to form LNFP-III and lactose, and
wherein the lactose is recycled by the cell to produce more 3FL.

95. The method according to any one of items 1 to 5151 or 54 to 73 or 78 to 91, wherein the complex oligosaccharide is LST-c and the method comprises the steps of:
a) cultivating a first genetically modified cell producing LNnT in a culture medium; and
b) supplying 6′SL to the culture medium of a); and
c) making an enzyme with transsialidase activity available in the culture medium; and
d) incubating the 6′SL, LNnT and the transsialidase enzyme in the culture medium to form LST-c and lactose; and
wherein the lactose is recycled by the cell to produce more LNnT.

96. The method according to any one of items 1 to 51 or 54 to 73 or 78 to 91, wherein the complex oligosaccharide is LST-a and the method comprises the steps of:
a) cultivating a first genetically modified cell producing LNT in a culture medium; and
b) supplying 3′SL to the culture medium of a); and
c) making an enzyme with transsialidase activity available in the culture medium; and
d) incubating the 3′SL, LNT and the transsialidase enzyme in the culture medium to form LST-c and lactose; and
wherein the lactose is recycled by the cell to produce more LNT.

97. The method according to any one of items 1 to 90, wherein the first genetically modified cell produces an acceptor oligosaccharide selected form the group consisting of 2′FL, 3FL, 2′FLacNAc, 2′FLNB, Lewis A, Lewis X, LNT-II, LNT, LNnT, Para-LNnH, LNFP-I, LNFP-II, LNFP-III, LNFP-IV, LNFP-V, LNFP-VI, 3′SL, 6′SL, LST-a, and LST-c.

98. The method according to any one of the preceding items, wherein the at least one recombinant glycosyltransferase in the genetically engineered cell is selected from the group consisting of α-1,2-fucosyltransferase, α-1,3-fucosyltransferase, α-1,3/4-fucosyltransferase, α-2,3-sialyltransferase, α-2,6-sialyltransferase β-1,3-N-acetylglucosaminyltransferase, β-1,6-N-acetylglucosaminyltransferase, β-1,3-galactosyltransferase, β-1,4-galactosyltransferase, β-N-acetylglucosaminidase and trans-β-galactosidase.

99. The method according to item 98, wherein the glycosyltransferase in the genetically engineered cell is an α-1,3-fucosyltransferase from table 8.

100. The method according to item 98 or 99, wherein the glycosyltransferase in the genetically engineered cell is the α-1,3-fucosyltransferase from Helicobacter pylori, such as the α-1,3-fucosyltransferase of SEQ ID NO: 1 or a functional homologue thereof.

101. The method according to item 98, wherein the glycosyltransferase in the genetically engineered cell is an α-1,2-fucosyltransferase from table 7.

102. The method according to item 98 or 101101, wherein the glycosyltransferase in the α-1,2-fucosyltransferase is from Helicobacter pylori.

103. The method according to item 98, wherein the glycosyltransferase in the genetically engineered cell is an α-2,3-sialyltransferase from table 10.

104. The method according to item 98, wherein the glycosyltransferase in the genetically engineered cell is an α-2,6-sialyltransferase from table 11.

105. The method according to item 98, wherein the at least one glycosyltransferase in the genetically engineered cell is β-1,3-N-acetylglucosaminyltransferase and a β-1,3-galactosyltransferase.

106. The method according to item 105, wherein the β-1,3-N-acetylglucosaminyltransferase is selected from table 4 and the β-1,3-galactosyltransferase is selected from table 5.

107. The method according to item 98, wherein the at least one glycosyltransferase in the genetically engineered cell is β-1,3-N-acetylglucosaminyltransferase and a β-1,4-galactosyltransferase.

108. The method according to item 107, wherein the β-1,3-N-acetylglucosaminyltransferase is selected from table 4 and the β-1,4-galactosyltransferase is selected from table 6.

109. The method according to any one of any one of the preceding items, wherein the pathway to produce a nucleotide-activated sugar is a pathway capable of producing at least one sugar nucleotide selected from the group consisting of GDP-fucose, UDP-GlcNAc, UDP-galactose, UDP-glucose, UDP-N-acetylglucosamine, UDP-N-acetylgalactosamine (GlcNAc) and CMP-N-acetylneuraminic acid

110. The method according to any one of any one of the preceding items, wherein the pathway to produce a nucleotide-activated sugar is the de novo GDP-fucose pathway (gmd, wcaG, manB, manC and manA) and/or the sialic acid sugar nucleotide pathway (neuB, neuC and neuA).

111. The method according to item 110, wherein the genetically modified cell overexpresses
a) the entire colonic acid gene cluster; and/or
b) one or more genes of the de novo GDP-fucose pathway selected from the group consisting of manA, manB, manC, gmd and wcaG.

112. The method according to item 110, wherein the genetically modified cell expresses a
a) recombinant UDP-GlcNAc 2-epimerase
b) recombinant Neu5Ac synthase; and
c) recombinant CMP-Neu5Ac synthetase.

113. The method according to any one of the preceding items, wherein the first genetically modified cell comprises a nucleic acid encoding a transporter protein capable of exporting the first oligosaccharide from the cell.

114. The method according to any one of items 10 to 113, wherein the second genetically modified cell comprises a nucleic acid encoding a transporter protein capable of exporting the second oligosaccharide from the cell.

115. The method according to item 113 or 114, wherein the transporter protein is a sugar efflux transporter or a major facilitator superfamily (MFS) transporter, preferably selected from the group consisting of setA, yberC, nec, vag, marc, bad and fred.

116. The method according to any one of the preceding items wherein the second disaccharide or oligosaccharide is selected from the group consisting of LacNAc, LNB, 2′FL, 3FL, 2′FLacNAc, 2′FLNB, Lewis A, Lewis X, 3′SL, LNT, LNnT, LNFP-I, LNFP-II, LNFP-III, LNFP-V, LNFP-VI, LSTa, LSTc, LNH, F-LNH-II, F-LNH-III, DF-LNH-I, DF-LNH-II, DF-LNH-III, S-LNH, DS-LNH, FS-LNH, LNnH, Para-LNH and Para-LNnH.

117. The method according to any one of the preceding items wherein the second oligosaccharide is selected from the group consisting of 2′FL, 3FL, 3′SL, LNT, LNnT, LNFP-I, LNFP-II, LNFP-III, LNFP-V, LNFP-VI, LST-a and LST-c.

118. The method according to item 116 or 117, wherein the second oligosaccharide is produced by a second genetically modified cell.

119. A composition of HMOs consisting essentially of:
a) at least 50 wt % FSL, below 45 wt % 3FL, and below 2 wt % 3′SL and below 3 wt % lactose, or
b) at least 60 wt % LNDFH-I, below 10 wt % LNFP-I, below 35 wt % 3FL and below 5 wt % lactose, or
c) at least 40 wt % LST-c, below 25 wt % LNnT, below 25 wt % 6′SL and below 10 wt % lactose, or
d) at least 55 wt % LST-a, below 40 wt % LNT, below 10 wt % 3′SL and below 2 wt % lactose, and
wherein the total composition constitutes 100 wt % of the components and the composition is a mixture of at least 2 components.

120. The composition according to item 119, wherein the composition consists essentially of
a) at least 50 wt % FSL, between 20 to 45 wt % 3FL, and between 0.1 to 2 wt % 3′SL and between 0 to 3 wt % lactose, or
b) at least 70 wt % LNDFH-I, between 0.1 to 8 wt % LNFP-I, between 15 to 25 wt % 3FL and between 0 to 3 wt % lactose
c) at least 50 wt % LST-c, between 15 to 25 wt % LNnT, between 15 to 25 wt % 6′SL and between 0 to 7 wt % lactose
d) e) at least 50 wt % LST-a, between 15 to 40 wt % LNT, between 0 to 15 wt % 3′SL and between 0 to 2 wt % lactose, and
   wherein the total composition constitutes 100 wt % of the components and the composition is a mixture of at least 2 components.

121. The composition according to item 119 or 120, wherein the molar ratio of FSL:3′SL is above 100:1 and the molar ratio of FSL:3FL is above 1:1.

122. The composition according to item 119 or 120, wherein the molar ratio of LNDFH-I:LNFP-I is above 50:1 and the molar ratio of LNDFH-I:3FL is in the range of 1.5:1 to 5:1.

123. The composition according to item 119 or 120, wherein the molar ratio of LST-a:LNT is above 1.5:1 and the molar ratio of LST-a:3′SL is above 9:1.

124. Use of the composition according to items 119 to 123 in the regeneration and viability of lyophilized probiotics.

125. A composition comprising a composition of HMOs according to items 119 to 123, wherein the composition further comprises one or more probiotics.

126. The composition according to item 125, wherein the probiotic is a Bifidobacterium sp and/or a Lactobacillus sp.

127. The composition according to 125 or 126, wherein the probiotic is selected from the group consisting of Bifidobacterium animals lactis BB12 DSM 32269, Bifidobacterium animals lactis BIF6, Bifidobacterium longum DSM 32946, Bifidobacterium longum BB536, Bifidobacterium bifidum DSMZ 32403, Bifidobacterium infantis, Bifidobacterium breve DSM 33789, Bifidobacterium infantis SP37 DSM 32687, Bifidobacterium adolescentis DSM 34065, Bifidobacterium animalis ssp. animalis DSM 16284, Lactobacillus rhamnosus GG DSM 32550, Lactobacillus rhamnosus 19070-2 DSM 26357, Lactobacillus rhamnosus GG, Lactobacillus rhamnosus LBrGG, Lactobacillus reuteri DSM 12246, Lactobacillus plantarum TIFN101, Lactobacillus gasseri Lg-36 200B FloraFit Danisco, Lactobacillus casei FloraActive PAR EVE DSMZ 32382, Lactobacillus paracasei, Lactobacillus plantarum PS 128, Lactobacillus plantarum (Sacco) DSM 32383, Lactococcus lactis PAREVE, Lactobacillus paracasei ssp. Paracasei and Lactobacillus Probio-Tec® LGG®, Limosilactobailius reuteri S12 DSM 33752

128. Use of the composition according to items 119 to 127 in the production of a nutritional composition.

129. The use according to item 128, wherein the nutritional composition is a dietary supplement and/or medical nutrition.

130. The use according to item 128, wherein the nutritional composition is in an infant nutrition.

### SEQUENCE LIST

The current application contains a sequence listing in text format and electronical format which is hereby incorporated by reference.

An overview of the SEQ ID NOs used in the present application are shown in the table below.

Summary of sequences listed in the application:

| Name | Comments | SEQ ID NO |
|---|---|---|
| FutA | ncbi sequence ID: NP_207177.1, alpha-1,3- | 1 |
| | fucosyltransferase from Helicobacter pylori 26695 | |
| marc | ncbi sequence ID: WP_060448169.1, MFS transporter | 2 |
| | protein from Serratia marcescens capable of exporting 3FL | |
| lacY | Lactose permease LacY, GenBank ID: NP_414877.1, | 3 |
| Transsialidases | Table 2 | 4-14, 40, 41 |
| | | and 60 |
| Transfucosidases | Table 3 | 15-39 |
| | | and 59 |
| PglpF promter | Promoter sequence | 42 |
| ST6, Pd2 | α-2,6-sialyltransferase | 43 |
| neuBCA | Nucleic acid sequence encoding the neuBCA cluster from | 44 |
| | Campylobacter jejuni | |
| IgtA | β-1,3-N-acetyl-glucosaminyltransferase | 45 |
| galT | β-1,4-Galactosyltransferase | 46 |
| ScrY | ncbi sequence ID: CAA40657.1, ScrY porin protein from | 47 |
| | Klebsiella pneumoniae | |
| ScrA | ncbi sequence ID: CAA40658.1, Sucrose transport protein | 48 |
| | Enzyme II scr protein from Klebsiella pneumoniae | |
| ScrB | ncbi sequence ID: WP_000056853.1, ScrB invertase | 49 |
| | enzyme from Salmonella enterica subsp. enterica serovar | |
| | Typhimurium | |
| ScrR | ncbi sequence ID: WP_000851062.1, Scr Repressor protein | 50 |
| | from Salmonella enterica subsp. enterica serovar | |
| | Typhimurium | |
| vag | GenBank accession ID WP_048785139.1, MFS transport | 51 |
| | protein capable of exporting LNnT | |
| cscABKR-gene | PTS dependent sucrose system from E. coli | 52 |
| cluster | | |
| PglF-SD1 promoter | Variant of the PglpF promoter sequence | 53 |
| Pscr promoter | Wildtype promoter of the from Klebsiella pneumoniae | 54 |
| Plac promoter | Wildtype promoter from E. coli | 55 |
| galTK | β-1,3-Galactosyltransferase | 56 |
| yberC | MFS transporter protein from Yersinia bercovieri capable of | 57 |
| | exporting LNT | |
| D29nst | α-2,3-sialyltransferase | 58 |
| Signal sequences | Signal sequences | 61-68 |

### EXAMPLES

### Methods

### Strains

The strains (genetically engineered cells) constructed in the present application were based on Escherichia coli K-12 DH1 with the genotype: F⁻, λ⁻, gyrA96, recA1, relA1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Methods of inserting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186: 2673-81 and Warming et al 2005 Nucleic Acids Res. 33(4): e36) with specific selection marker genes and screening methods.

This MDO strain was further engineered to generate a strain the produce the desired HMO. For example, the 3FL producing strain was generated by exchanging the native promoter in front of the colonic acid cluster with a PglpF promoter (wcaF::PglpF). Furthermore, the strain has three copies of the futA gene under control of the PglpF promoter encoding the α-1,3-fucosyl-transferase of SEQ ID NO: 1 and one copy of marc gene under control of the PglpF promoter encoding the MFS transporter Marc of SEQ ID NO: 2. The geneotypes of all the strains used in the present examples are indicated in table 12.

TABLE 12
genotypes of the E. coli strains used in the examples

| Strain ID | Product | Genotype | Plasmid |
|---|---|---|---|
| MDO | None | F− λ− ΔendA1 ΔrecA1 ΔrelA1 ΔgyrA96 Δthi-1 | None |
| | | glnV44 hsdR17(rK−mK−) ΔlacZ wcaF::Plac | |
| | | ΔnanKETA ΔlacA ΔmelA ΔwcaJ ΔmdoH | |
| 3FL-S1 | 3FL | MDO wcaF::PglpF⁸ 3xPglpF-futA⁹ PglpF-marc¹⁰ | None |
| | | ΔlacI¹⁷ | |
| 3FL-S2 | 3FL | MDO MDO wcaF::PglpF⁸ 3xPglpF-futA⁹ PglpF-marc¹⁰ | None |
| | | ΔlacI¹⁷ ΔrpoS¹⁸ | |
| 6′SL-S1 MF5 | 6′SL | MDO PglpF-ST6, Pd2¹ ΔnadC² | pBS-nadC-Plac- |
| | | | neuBCA³ |
| LNnT-S1 | LNnT | MDO 2x PglpF-IgtA⁴, PglpF-galT⁵, PglpF_SD1-Pscr- | None |
| MF6 | | scrYA⁶, Pscr-scrBR⁶, PglpF-vag⁷, ΔptsG¹¹ ΔlacI¹⁷ | |
| LNnT-S2 | LNnT | MDO 2x PglpF-IgtA⁴, PglpF-galT⁵, PglpF_SD1-Pscr- | None |
| MF7 | | scrYA⁶, Pscr-scrBR⁶, PglpF-vag⁷ PglpF-LacY¹⁴ | |
| | | ΔlacI¹⁷ | |
| LNT-S1 | LNT | MDO: PglpF-galTK¹², 2x PglpF-IgtA⁴, Plac-YberC¹³, | None |
| | | PglpF_SD1-Pscr-scrYA⁶, Pscr-scrBR⁶, PglpF- | |
| | | LacY¹⁴, ΔlacI¹⁷ Δglf¹⁵, ΔptsG¹¹ | |
| 3′SL-S1 | 3′SL | MDO, 2xPlac-D29nst¹⁶, ΔnadC² ΔlacI¹⁷ | pBS-neuBCA- |
| | | | wt-nadC |

¹ST6, Pd2 - one genomic copy of a gene encoding α-2,6-sialyltransferase of SEQ ID NO: 43 under control of a PglpF promoter (SEQ ID NO: 42).
²ΔnadC - deletion of quinolinate phosphoribosyl-transferase of WP_101348535.1 for further details see WO2017/101958.
³pBS-nadC-Plac-neuBCA - plasmid expressing neuBCA gene cluster (SEQ ID NO: 44) and nadC further details see WO 2017/101958.
⁴lgtA-PglpF - two genomically inserted copies of a gene encoding β-1,3-N-acetyl-glucosaminyltransferase (SEQ ID NO: 45) under control of a PglpF promoter (SEQ ID NO: 42).
⁵galT-PglpF - one genomically inserted gene encoding β-1,4-Galactosyltransferase (SEQ ID NO: 46) under control of a PglpF promoter (SEQ ID NO: 42).
⁶scrYA, scrBR two operons encoding the sequences of SEQ ID NO: 47 and 48 and SEQ ID NO: 49 and 50 respectively, and under the control of the PglpF_SD1 promoter (SEQ ID NO: 53) and Pscr promoter (SEQ ID NO: 54), respectively,
⁷vag - one genomic inserted copy of a gene encoding MFS transporter of SEQ ID NO: 50 under control of a PglpF promoter (SEQ ID NO: 42).
⁸wcaF::PglpF insertion of a PglpF promoter (SEQ ID NO: 42) in front of the endogenous colonic acid operon. This can also be written as CA::CA(PglpF).
⁹FutA - three individually genomically inserted copies of gene encoding α-1,3-fucosyltransferase (SEQ ID NO: 1) under control of a PglpF promoter (SEQ ID NO: 19).
¹⁰marc - one genomic inserted copy of a gene encoding MFS transporter (SEQ ID NO: 2) under control of a PglpF promoter (SEQ ID NO: 42).
¹¹ΔptsG - deletion of the native glucose PTS complex component IICB^{Glc} - impairs the strains ability to grow on glucose.
¹²PglpF-galTK - one genomically inserted copy of a gene encoding the beta-1,3-galactosyltransferase of SEQ ID NO: 56 under control of a PglpF promoter (SEQ ID NO: 42).
¹³Plac-YberC - one genomic inserted copy of a gene encoding the MFS transporter of SEQ ID NO: 57 under control of a PglpF promoter (SEQ ID NO: 42)
¹⁴PglpF-LacY - one additional genomic inserted copy of a gene encoding E. coli LacY transporter of SEQ ID NO: 3 under control of a PglpF promoter (SEQ ID NO: 42).
¹⁵Δglf - deletion of the native glucose facilitator - impair the strains growth on glucose.
¹⁶Plac-D29nst - two individually inserted genomic copies of a gene encoding the α-2,3-sialyltransferase of SEQ ID NO: 58 under control of a Plac promoter (SEQ ID NO: 55).
¹⁷ΔlacI - Deletion of lac repressor makes use of IPTG obsolete.
¹⁸ΔrpoS - deletion of sigma factor S - increase product yield (see PCT/EP2023/065491 for further details).

### Fermentation

The fermentation-enzymatic hybrid process was carried out in a 2L Sartorious B-stat bioreactor starting with 700-1000 g of sterile mineral culture medium consisting of glucose and/or sucrose as carbon source (sterilized separately), lactose monohydrate (sterilized separately) in an amount sufficient to produce the desired amount of oligosaccharide produced by the cell, however not more than what the cell can convert into the desired oligosaccharide produced by the cell, e.g. in the range from 10-80 g/kg, (NH₄)₂HPO4, KH₂PO₄, MgSO₄×7H₂O (sterilized separately), KOH, NaOH, citric acid, trace element solution, antifoam and thiamine (added by sterile filtration). The dissolved oxygen level was kept above 20%, such as at 23%, by a cascade of first agitation (1000-2000 rpm) and then airflow (1-3 VVM). The pH was kept at 6.8 by titration with NH₄OH solution. The cultivation was started with a 2% (v/v) inoculum from a pre-culture grown to an OD₆₀₀ of 2.5-5 in similar glucose and/or sucrose containing medium.

After depletion of the carbon source(s) contained in the batch medium, sterile mineral feed solution containing a predefined amount of carbon source (glucose and/or sucrose), MgSO₄×7H₂O, H₃PO₄, trace metals and antifoam was fed continuously using a predefined feed profile that kept the cultivation carbon limited with fully respiratory metabolism. In addition, in some examples a sterilized 2^{nd} HMO (defined in the examples below) was added either via a feed profile or from the beginning of the fermentation. The temperature was initially set to 33 or 34° C. and then dropped to a preset temperature between 30° C. and 25° C. (defined in the individual examples below). The growth, and state of the cells were followed by biowetmass (weight of cell pellet after 3 min centrifugation at 14,000 g/weight of broth), optical density at 600 nm and on-line measurements of CO₂ evolution rate, agitation, NH₄OH base addition, dissolved oxygen and temperature. Lactose and oligosaccharide concentrations were determined once or several times per day via HPLC. Samples for HPLC analysis were heat treated at 90° C. for 20 min at the time of collection to stop the enzymatic process.

### Example 1 Synthesis of FSL Using a Fermentation-Enzymatic Hybrid Process with a 3FL Strain and Addition of α-2,3-Transsialidase and Pure 3′SL to the Broth

The present example illustrates that it is possible to form FSL directly in a fermentation broth with a cell producing 3FL from lactose and by adding a α-2,3-transsialidase and the second substrate 3′SL to the broth.

In the present example the 3FL strain was fermented as described in the Methods section above starting with 700 g of medium after the addition of the inoculum. After an initial batch phase of 14 h, the fed-batch phase was started by continuously feeding a mineral containing glucose solution with an initial glucose feed rate of 1.35 g glucose/h with a linear ramp up to a feed rate of 2.7 g/h over 5 hours, whereafter it was kept constant. 3′SL was fed with a feed profile corresponding to a fraction of the glucose feed rate that reflected the 3FL strains' yield on glucose to roughly match the accumulation rate of 3′SL with 3FL. 24 hours after feed start the fermentation temperature was dropped with a linear ramp over 5 h to 25° C. The trans-sialylation of 3FL was started by adding 115 mg/L fermentation broth of sterile filtered α-2,3-transsialidase (TcTS, SEQ ID NO: 60) enzyme at 89 h and additional 0.175 mg/ml TcTS at 118 h. The fed-batch continued until 139 h after fermentation start. Moreover, the 3′SL (2^{nd} HMO) feeding was stopped at 119 h after fermentation start.

The 3FL formation from lactose, formation of FSL as well as consumption of 3′-SL were monitored by measuring the components via HPLC. Samples for HPLC analysis were heat treated at the time of collection to stop the enzymatic process. FIG. 2 illustrates the progress of the process in weight % relative to the total weight of substrates and products. As can be seen in FIG. 2, at 89 h (i.e. the addition of the α-2,3-transsialidase enzyme) nearly all lactose was consumed by the cell to form 3FL while 3′SL was fed into the system proportional to the mass of 3FL formation. The trans-sialylation of 3FL using 3′SL as a sialyl donor to form FSL was initiated by adding α-2,3-transsialidase enzyme at 89 h of the process and can be seen in FIG. 2 as the formation of FSL and decrease of 3′SL. The trans-sialyation process consumes 3FL and 3′SL and forms lactose in equimolar amounts as a side-product. The lactose was in turn taken up and re-cycled into 3FL by the E. coli strain, therefore, after the start of the enzymatic reaction a near steady-state was achieved with respect to lactose and 3FL level since 1:1 molar stochiometric amount of 3FL was consumed in the FSL reaction together with the formation of 1:1 molar amounts of the side-product lactose, which was recycled back in a 1:1 molar ratio to 3FL. FSL on the other hand increased until near complete conversion of 3′SL. Thus, 82% yield of FSL relative to the total added amount of 3′-SL was achieved at the end of the hybrid process while the 3′-SL conversion was 99.5%. As can be observed there was not a 100% conversion of 3′-SL to FSL, this may be due to factors such as the stability of 3′SL during heat sterilization and storage, and/or that the enzyme does not have 100% trans-sialylation selectivity due to its side hydrolytic activity. Moreover, with the full conversion of 3′SL and the recycling of lactose to the formation of 3FL, the hybrid process generated a final solution consisting of 3FL and FSL as the major HMOs with very low amount of residual lactose.

Example 2 is a comparative example of an in vitro process (strictly enzymatic) applying the α-2,3-transsialidase enzyme to purified 3FL and 3′SL. Table 13 below shows the relative yield of each component at the end of either the in vitro or hybrid process as well as the conversion rate of the sialyl donor (3′SL) and the FSL yield relative to 3′SL.

TABLE 13
Comparison of the final product composition of the hybrid
process (example 1) vs. the in vitro process (example
2) for the synthesis of FSL, 3′SL conversion and
FSL yield are shown as relative to 3′SL addition.

| | | | | | 3′SL | FSL yield |
|---|---|---|---|---|---|---|
| | Lactose | 3FL | 3′SL | FSL | conversion | relative |
| Process | (wt. %) | (wt. %) | (wt. %) | (wt. %) | (%) | to 3′SL (%) |
| Hybrid | 1.2 | 43.8 | 0.3 | 54.7 | 99.5 | 82.1 |
| In vitro | 18.8 | 32.5 | 16.2 | 32.5 | 69.1 | 50.2 |

The comparison shows that the hybrid process has a significantly higher FSL yield relative to 3′SL compared to the in vitro process. In addition, the amount of 3′SL and lactose remaining in the final product composition was very low for the hybrid process. This shows that the hybrid process offers a further purification advantage as isolating FSL from 3FL is easier than from 3′SL due to the difference in electrostatic charge (FSL and 3′SL being negatively charged in conjugated base form, whereas 3FL is neutral).

### Example 2—Comparative Example—Synthesis of FSL Using an In Vitro Process

The formation of sialyloligosaccharides using an in vitro trans-sialyation reaction is known from for example WO 2012/007588. In the present example such a reaction was performed using pure 3FL and 3′SL as acceptor and donor, respectively to illustrate the difference between the in vitro system and the hybrid system in Example 1.

An in vitro synthesis of FSL was carried out using the α-2,3-transsialidase enzyme (TcTS, SEQ ID NO: 60) from Example 1 to catalyze trans-sialylation of 3FL utilizing 3′SL as a sialyl donor. A substrate solution consisting of 107.33 mM 3FL and 107.33 mM 3′SL, pH 6.85 was prepared. The reaction was started by adding 1 mg/ml TcTS at 25° C. The reaction progress was monitored by measuring the concentrations of substrates and products over time via HPLC. Samples for HPLC measurements were collected by treating them at 95° C. for 5 min to denaturing the TcTS enzyme thereby stopping the reaction. Table 14 shows the relative amount of the different products in relation to the total amount of products developing over time.

TABLE 14
Relative amounts of individual components over time
for the in vitro synthesis of FSL catalyzed by α-2,3-
transsialidase from 3FL and 3′SL

| | | | | | 3′SL | FSL yield |
|---|---|---|---|---|---|---|
| Time | Lactose | 3FL | 3′SL | FSL | conversion | relative |
| (h) | (wt. %) | (wt. %) | (wt. %) | (wt. %) | (%) | to 3′SL (%) |
| 0.0 | 2.5 | 21.2 | 26.2 | 0.0 | 0.0 | 0.0 |
| 0.3 | 3.8 | 21.6 | 25.6 | 0.7 | 2.5 | 3.0 |
| 0.5 | 3.3 | 21.3 | 25.9 | 1.2 | 5.3 | 5.5 |
| 1.5 | 5.3 | 19.8 | 23.9 | 2.6 | 10.3 | 12.3 |
| 2.5 | 6.7 | 18.0 | 20.9 | 3.3 | 13.9 | 17.1 |
| 4.0 | 8.0 | 17.1 | 19.9 | 4.3 | 18.2 | 22.4 |
| 6.0 | 8.0 | 16.5 | 19.4 | 5.1 | 22.8 | 26.7 |
| 51.3 | 19.0 | 11.6 | 11.4 | 10.6 | 45.6 | 54.6 |

From the results in table 14 it can be seen that as the 3′SL concentration decreased the FSL concentration increased, indicating that 3′SL was converted into FSL. Moreover, as the sialyl is removed from the 3′SL the lactose concentration also increased. The last column of table 2 show the FSL yield relative to the starting amount of 3′SL. At the equilibrium of the reaction the FSL yield relative to the starting 3′SL amount was about 54%.

### Example 3: Synthesis of LNDFH-I Using a Hybrid Process

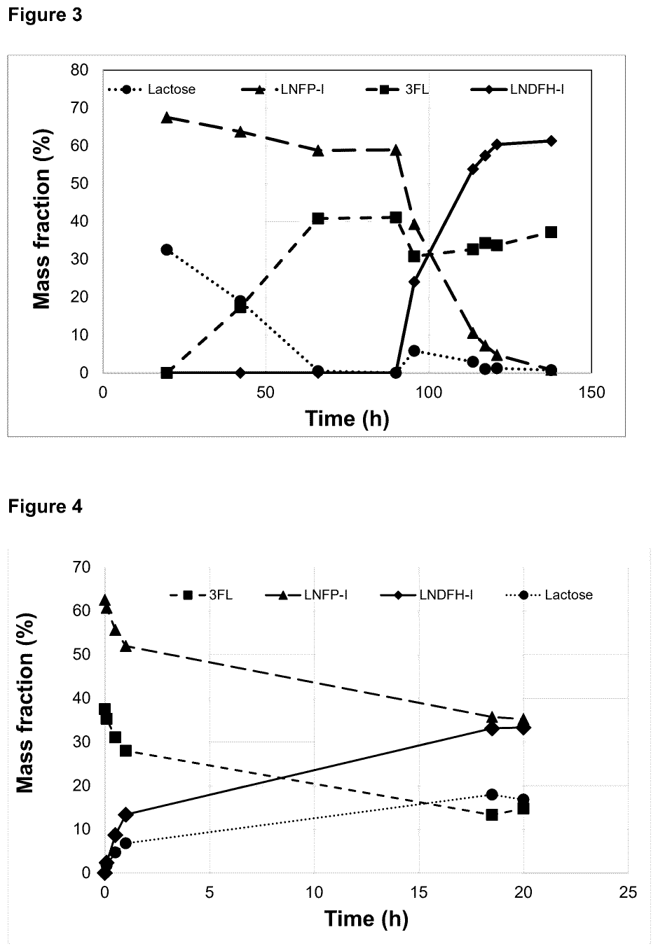

The synthesis of LNDFH-I using the hybrid process was performed with the 3FL producing strain, 3FL-S1, from table 12 by fermentation from lactose starting with 1000 g of medium after the addition of the inoculum. In the present example the 3FL strain was fermented as described in the Methods section above. After an initial batch phase of 14 h, the fed-batch phase was started by continuously feeding a mineral containing glucose solution with an initial feed rate of 2.19 g glucose/h with a linear ramp up to a feed rate of 4.37 g/h linearly over 5 hours, whereafter it was kept constant. 3 hours after feed start the fermentation temperature was dropped with a linear ramp over 1 h to 30° C. In this example equimolar amount of lactose and LNFP-I was added at the start of the fermentation. The trans-fucosylation of LNFP-I was started by adding 68 mg/L fermentation broth of sterile filtered mutated recombinant α1,3/4-transfucosidase from Bifidobacterium longum subsp. Infantis (BiTF-641, SEQ ID NO: 59) at 90 h after the start of the fermentation. In this hybrid process the formation of LNDFH-I by transfucosylation in the culture broth of the acceptor molecule (LNFP-I) using the in vivo formed 3FL (from lactose) as a fucosyl donor, was monitored by measuring the individual components via HPLC. Samples for HPLC analysis were heat-treated at the time of collection to stop the enzymatic process. FIG. 3 illustrates the development of lactose, LNFP-I, 3FL and LNDFH-I in weight % relative to the total weight of the substrates and products. As can be seen from FIG. 3, until 90 h into the process (i.e., the addition of the enzyme) no formation of LNDFH-I occurred, whereas the lactose was fully converted to 3FL by the recycling activity of the genetically modified cell. The transfucosylation of LNFP-I utilizing 3FL as a fucosyl donor was initiated by adding the BiTF-641 at 90 h to the process. LNDFH-I increased after the addition of enzyme and a molar equivalent decrease of the LNFP-I was observed. Following the initiation of the transfucosylation reaction the concentration of lactose increased as it was released from the fucosyl-donor 3FL, but it was subsequently taken up by the cell and again converted into 3FL illustrating the recycling of lactose. As can be seen in FIG. 3, the recycling of lactose to the in-situ formation of 3FL circumvented the trans-fucosylation kinetic equilibrium barrier and led to a 98.7% conversion of LNFP-I to LNDFH-I.

Example 4 is a comparative example of an in vitro process (strictly enzymatic) applying the α1,3/4-transfucosidase enzyme purified 3FL and LNFP-I. Table 15 below shows the relative composition of each component at the end of the in vitro and the hybrid processes as well as the conversions of LNFP-I.

TABLE 15
Comparison of the final product composition and LNFP-I conversion
of the hybrid vs in vitro process for the synthesis of
LNDFH-I starting at equimolar acceptor (LNFP-I) to donor
(lactose for hybrid or 3FL for the in vitro) ratio.

| | | | | | LNFP-I |
|---|---|---|---|---|---|
| | Lactose | 3FL | LNFP-I | LNDFH-I | conversion |
| Process | (wt. %) | (wt. %) | (wt. %) | (wt. %) | (%) |
| Hybrid | 0.73 | 31.2 | 0.81 | 61.3 | 98.7 |
| In vitro | 16.8 | 14.7 | 35.2 | 33.3 | 42.5 |

Only the conversion of LNFP-I is used as a process comparison metrics as the 3FL is continuously formed due to the recycling of lactose. The hybrid process not only led to a higher conversion of LNFP-I but also a final product mixture with a very low level of lactose. Thus, the hybrid process offers the advantage of obtaining the final product consisting of 3FL and LNDFH-I as major components with a very low content of lactose and LNFP-I as shown in table 15. This is an advantage if it is desired to obtain pure LNDFH-I since it is easier to separate 3FL and LNDFH-I than LNFP-I and LNDFH-I, due to the larger difference in molecular size. In addition, the hybrid process offers the advantage of replacing the expensive starting material 3FL with cheap lactose and offering a much higher conversion of the even more expensive starting material LNFP-I compared to the in vitro process, thus in all constituting a much more economical process.

### Example 4: Comparative Example—Synthesis of LNDFH-I Using an In Vitro Process

An in vitro synthesis of LNDFH-I was carried out using BiTF-641 (SEQ ID NO: 59) catalyzed transfucosylation of LNFP-I utilizing 3FL as a fucosyl donor by preparing a substrate solution consisting of equimolar donor and acceptor substrates. The substrate solution consisting of 150 mM LNFP-1 and 150 mM 3FL was prepared at pH 6.55. The transfucosylation reaction was started by adding 0.51 mg/ml BiTF-641 at 25° C. The reaction progress was monitored by measuring the concentrations of substrates and products via HPLC. Samples for HPLC were collected by treating them at 95° C. for 5 min to denature the BiTF-641 enzyme thereby stopping the reaction. FIG. 4 shows the reaction progress curve of the in-vitro synthesis of LNDFH-I in weight % relative to the total weight of substrates and products and indicate that the reaction had reached its equilibrium. The final product composition and LNFP-I conversion of the in vitro process vs. hybrid process for the synthesis of LNDFH-I starting at equimolar acceptor (i.e. LNFP-1) to donor (i.e. lactose for hybrid or 3FL for the in vitro) ratio is shown in table 15 in example 3.

As can be seen in FIG. 4 in contrast to the hybrid process which led to a very low steady lactose concentration and 98.7% conversion of LNFP-I (i.e., shown at FIG. 3), the in vitro process led to at least a stochiometric equimolar or more amount of lactose with LNDFH-1 and 42.5% conversion of LNFP-I. Typically more than a stochiometric amount of lactose relative to LNDFH-I is formed in the in vitro process due to the side hydrolytic activity of the BiTF-641 enzyme, cleaving 3FL thereby increasing the lactose level without the formation of LNDFH-I.

### Example 5: Synthesis of LNDFH-I Using a Hybrid Process Starting at High Acceptor to Donor Ratio

The synthesis of LNDFH-I in the hybrid process was performed using the 3FL-S1 strain from table 12 by fermentation from lactose, starting with 900 g of medium after the addition of the inoculum.

In the present example the 3FL strain was fermented as described in the Methods section above. After an initial batch phase of 14 h, the fed-batch phase was started with an initial feed rate of 1.96 g glucose/h with a linear ramp up to a feed rate of 3.92 g/h linearly over 5 hours, whereafter it was kept constant. 3 hours after feed start the fermentation temperature was dropped with a linear ramp over 1 h to 30° C. In this example the total amount of lactose used in the process was added at the start of the fermentation whereas the LNFP-I was fed at a fixed rate starting 15 hours into the fed-batch phase and lasting for 48 h. The trans-fucosylation of LNFP-I was started by adding 112 mg/L fermentation broth of sterile filtered mutated recombinant α1,3/4-transfucosidase from Bifidobacterium longum subsp. Infantis (BiTF-641, SEQ ID NO: 59) at 88 h after the start of the fermentation. In this hybrid process the formation of LNDFH-I by transfucosylation of the acceptor molecule LNFP-I in the culture broth using the in vivo formed 3FL (from lactose) as a fucosyl donor was monitored over time by measuring the individual components via HPLC. Samples for HPLC analysis were heat treated at the time of collection to stop the enzymatic process.

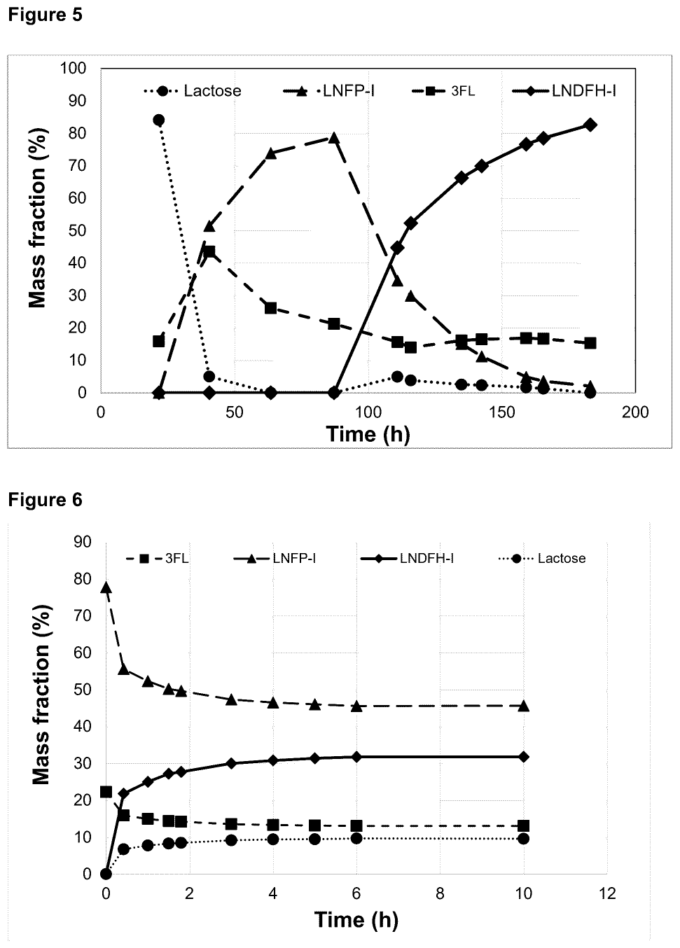

FIG. 5 illustrates the progress of the process in weight % relative to the total weight of substrates and products. Until 88 h of the process (i.e., the time of addition of the enzyme) no formation of LNDFH-I occurred but full consumption of lactose substantiated by the in-vivo formation of 3-FL was observed. The amount of LNFP-I increased until the feed was stopped and dropped rapidly after the addition of the enzyme. As expected, the LNDFH-1 level increased after the addition of enzyme with a corresponding decrease of LNFP-I (acceptor). Whereas the levels of lactose and 3FL remained almost constant due to the recycling of lactose released from the transfucosylation reaction to the formation of 3FL. The recycling of lactose for the in situ formation of 3FL circumvented the transfucosylation kinetic equilibrium barrier which led to a 97.8% conversion of LNFP-I to LNDFH-I.

Example 6 is a comparative example of an in vitro process (strictly enzymatic) applying the α1,3/4-transfucosidase enzyme to purified 3FL and LNFP-I. Table 16 below shows the relative composition of each component at the end of the in vitro and the hybrid processes as well as the conversions of LNFP-I.

TABLE 16
Comparison of final product composition of the hybrid process
vs. in-vitro process for the synthesis of LNDFH-I starting
at an acceptor (LNFP-I) to donor (3-FL) molar ratio of 2.

| | | | | | LNFP-I |
|---|---|---|---|---|---|
| | Lactose | 3FL | LNFP-I | LNDFH-I | conversion |
| Process | (wt. %) | (wt. %) | (wt. %) | (wt. %) | (%) |
| Hybrid | 0.00 | 15.3 | 2.06 | 82.6 | 97.8 |
| In vitro | 9.57 | 13.1 | 45.6 | 31.8 | 37.0 |

Only the conversion of LNFP-I is used as a process comparison metrics as the 3FL was continuously formed due to the recycling of lactose.

From table 16 it can be seen that the hybrid process not only led to a higher conversion of LNFP-I but also to a final product mixture with a very low level of lactose and LNFP-I. Thus, the hybrid process offers the advantage of obtaining a final mixture consisting of 3FL and LNDFH-I as major components with a very low content of lactose and LNFP-I as shown in table 16. This is an advantage if it is desired to obtain pure LNDFH-I since it is easier to separate 3FL and LNDFH-I than LNFP-I and LNDFH-I. In addition, the hybrid process offers the advantage of replacing the expensive starting material 3FL with cheap lactose and offering a much higher conversion of the even more expensive starting material LNFP-I compared to the in vitro process, thus in all constituting a much more economical process.

Example 3 and 5 were conducted to investigate the effect of excess of the second oligosaccharide (in this case the acceptor) over the first oligosaccharide in the hybrid process and example 4 and 6 does the same in the in vitro process. For the hybrid process the acceptor to donor ratio is essentially between LNFP-I and lactose, since the lactose is fully converted to 3FL. Table 17 compiles the results from table 15 and table 16.

TABLE 17
Comparison of final product composition of the hybrid process
vs. in-vitro process for the synthesis of LNDFH-I starting
at different acceptor (LNFP-I) to donor (3-FL) molar ratio

| | Acceptor/ | | | | | |
|---|---|---|---|---|---|---|
| | Donor | | | LNFP- | LNDFH- | LNFP-I |
| | molar | Lactose | 3FL | I (wt. | I (wt. | conversion |
| Process | ratio (—) | (wt. %) | (wt. %) | %) | %) | (%) |
| Hybrid | 1 | 0.73 | 31.17 | 0.81 | 61.29 | 98.7 |
| Hybrid | 2 | 0 | 15.3 | 2.06 | 82.64 | 97.8 |
| In-vitro | 1 | 16.77 | 14.74 | 35.21 | 33.28 | 42.5 |
| In-vitro | 2 | 9.57 | 13.05 | 45.61 | 31.78 | 37.0 |

The results clearly illustrate the positive effect of increasing the amount of the second oligosaccharide (fed to the culture (acceptor)) to first oligosaccharide (produced by the strain (donor)) in the hybrid process where the LNDFH-I level was increased by 35% and the 3FL level was reduced by 50% when the acceptor was in excess. For the in vitro process the increased acceptor:donor ratio did not result in any positive effect on the LNDFH-I formation and the LNFP-1 level remained significantly higher than both 3FL and lactose which illustrates that it is limited by the kinetic barrier of the enzymatic reaction. While in the hybrid process, avoiding the limitation of the oligosaccharide that is added to the process (the second oligosaccharide) allows for an increased formation of the third oligosaccharide since the 1^{st} oligosaccharide (LNFP-I) will continuously be produced as long as the 2^{nd} oligosaccharide (3FL) is present and lactose is produced from the transfucosylation reaction thereby continuously feeding the strain with initial substrate for renewed LNFP-I formation and removing the kinetic barrier observed in the in vitro process.

### Example 6: Comparative Example—Synthesis of LNDFH-I Using an In Vitro Process Starting at High Acceptor to Donor Ratio

An in vitro synthesis of LNDFH-I was carried out using BiTF-641 (SEQ ID NO: 59) catalyzed trans-fucosylation of LNFP-I utilizing 3FL as a fucosyl donor starting at 2:1 mol/mol LNFP-I:3FL ratio. A substrate solution consisting of 200 mM LNFP-I and 100 mM 3FL was prepared at pH 6.5. The trans-fucosylation reaction was started by adding 0.25 mg/ml BiTF-641 at 37° C. The reaction progress was monitored by measuring the concentrations of substrates and products via HPLC. Samples for HPLC were collected by treating them at 95° C. for 5 min to denature the BiTF-641 enzyme thereby stopping the reaction.

FIG. 6 shows the reaction progress curve of the in-vitro synthesis of LNDFH-I at starting at an acceptor to donor molar ratio of 2:1, and clearly shows the reaction reaching thermodynamic equilibrium.

As can be seen in FIG. 6 in contrast to the hybrid process which led to very low and steady lactose and 3FL concentrations and 97.8% conversion of LNFP-I (i.e., shown in FIG. 5), the in vitro process leads to at least a stochiometric equimolar amount of lactose compared to LNDFH-I and a limited 37% conversion of LNFP-I. The results are summarized in Table 17.

### Example 7 Synthesis of LST-c Using a Two-Strain Hybrid Process

In the present example two strains (see table 12), LNnT-S1 producing LNnT from lactose (substrate) and sucrose (carbon source) and 6′SL-S1 producing 6′SL from lactose (substrate) and glucose (carbon source) were co-cultured. The LNnT-S1 has reduced growth on glucose due to deletion of the ptsG transporter. The 6′SL-S1 strain cannot grow on sucrose. 6′SL was produced as sialyl donor substrate and LNnT as acceptor substrate for a subsequent trans-sialyation reaction. LST-c was formed in the fermentation medium by addition of a α2,6-transsialidase to the fermentation medium. This process is also termed a two-strain hybrid process.

### Fermentation

The 2-strain hybrid process was carried out in a 2 L Sartorious B-stat bioreactor starting with 1000 g of mineral culture medium consisting of 15 g/kg glucose (sterilized separately) and 15 g/kg sucrose (sterilized separately), lactose monohydrate (sterilized separately), (NH₄)₂HPO₄, KH₂PO₄, MgSO₄×7H₂O (sterilized separately), KOH, NaOH, citric acid, trace element solution, antifoam and thiamine (filter sterilized). The dissolved oxygen level was kept at 20% by a cascade of first agitation and then airflow of 1 VVM (up to max 3 VVM). The pH was kept at 6.8 by titration with NH₄OH solution. The cultivation was started with 1% (v/v) inoculums from each pre-culture grown in a similar containing medium to an OD₆₀₀ of 2.5-5, with glucose as carbon source for the 6′SL strain and sucrose as carbon source for the LNnT strain. After depletion of the glucose and sucrose contained in the batch medium after approximately 14 h, a mineral feed solution containing glucose and sucrose at a 1:2 ratio (w/w) (sterilized together separately from the minerals), MgSO₄×7H₂O, H₃PO₄, trace metals and antifoam, was fed continuously using a constant profile that provided 1 g/h glucose and 2 g/h sucrose, which kept the culture carbon limited. The temperature was initially set to 34° C. but was dropped to 28° C. with a linear 1 h ramp after 3 hours of feed. The growth, and state of the cells were followed by biowetmass (weight of cell pellet/weight of broth after 3 min centrifugation at 14,000 g), optical density at 600 nm and on-line measurements of CO₂ evolution rate, agitation, base addition, dissolved oxygen and temperature.

The transsialylation was started by adding 3390 mg/L fermentation broth of α-2,6-transsialidase from Photobacterium leiognathi JT-SHIZ-119 (PITS-197, SEQ ID NO: 41) at 113 h after fermentation start, when almost all the lactose had been converted into 6′SL and LNnT. The process was monitored via HPLC by measuring the concentrations of substates and product.

### Results

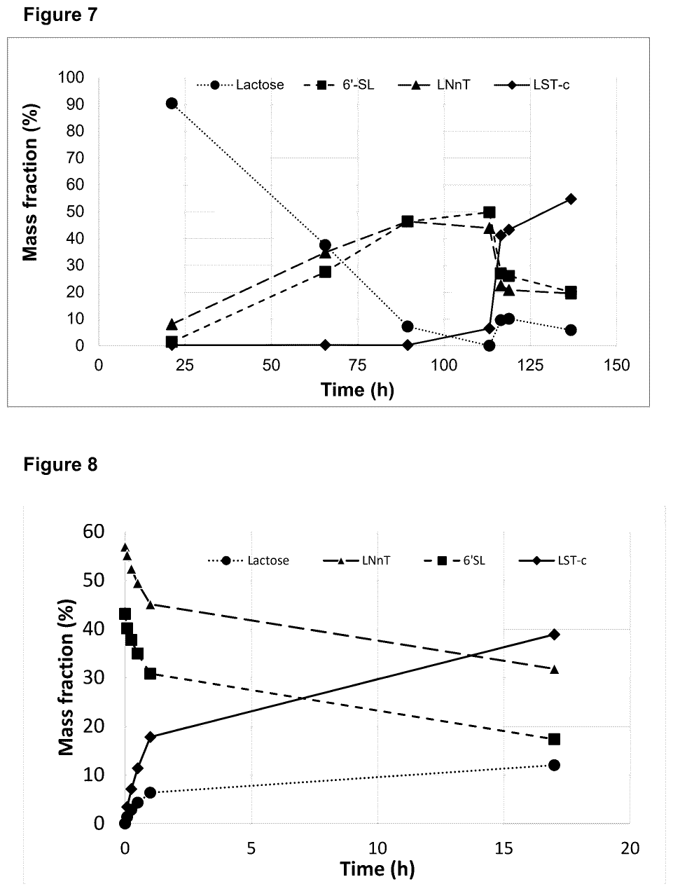

FIG. 7 shows concentration data illustrating the process progress in weight % relative to the total weight of substrates and products.

From this it can be seen, that up until 113 h where all the lactose is consumed and the transsialidase was added to the culture, the LNnT and 6′SL strains produced 6′SL and LNnT in close to equimolar ratios. Upon the addition of the transsialidase the LST-c formation was initiated by consuming 6′SL and LNnT as can be seen by their decreasing amounts. The formation of LST-c also generates an equimolar amount of lactose as the leaving group from the transsialyation reaction. However, as the lactose was taken up by the LNnT and 6′SL strains and reutilized to form 6′SL and LNnT its level was kept low.

This example shows (see FIG. 7) that the second oligosaccharide can be obtained from a second genetically modified organism which is co-cultured with the genetically modified organism producing the first oligosaccharide (the genetically modified strain). The two strains were co-cultured on two different carbon sources which allowed for stable formation of both the first and the second oligosaccharide that were utilized in the trans-sialylation reaction to form the complex sialylated oligosaccharide. It also shows that large differences in product yields (g LNnT or 6′SL pr g of carbon source) of two strains can be compensated by feeding them at a modified carbon source ratio to achieve a desired product ratio, in this case a glucose:sucrose ratio of 1:2 (g/g) to achieve a ratio of 6′SL:LNnT of 1:1.

As in the above examples for the one strain hybrid process, the two-strain hybrid process was also compared to the conventional in-vitro enzymatic process.

The conventional in vitro enzymatic process was conducted using the same α-2,6-transsialidase PITS-197 which catalyzed transsialylation of LNnT utilizing 6′SL as a sialyl donor. A substrate solution was prepared with 116.8 mM LNnT and 116.8 mM 6′SL at pH 6.87. The transsialylation reaction was started by adding 1.57 mg/ml PITS-197 at 25° C. The reaction progress was monitored by measuring the concentrations of substrates and products via HPLC. Samples for HPLC measurements were collected by treating them at 95° C. for 5 min to denature the PITS-197 enzyme thereby stopping the reaction. FIG. 8 shows the reaction progress curve of the in-vitro synthesis of LST-c. As can be seen in FIG. 8 in contrast to the two-strain hybrid process which lead to a very low lactose concentration (shown in FIG. 7), the in-vitro process leads to an equimolar amount of lactose with LST-c. Table 18 shows the comparison of the final product composition of LST-c synthesis using the two-strain hybrid vs. in vitro processes.

TABLE 18
Comparison of final product composition of the two-strain hybrid
process vs. in vitro process for the synthesis of LST-c starting
at an equimolar acceptor (LNnT) to donor (6′SL) ratio.

| | Lactose | 6′-SL | LNnT | LST-c |
|---|---|---|---|---|
| Process | (wt. %) | (wt. %) | (wt. %) | (wt. %) |
| In vitro | 11.9 | 17.3 | 31.8 | 38.8 |
| Two-strain hybrid | 5.8 | 20.0 | 19.5 | 54.6 |

Table 18 shows that the LST-c formation was significantly increased in the two-strain hybrid process resulting from a higher conversion of LNnT. In addition, the final product mixture obtained from the two-strain hybrid process had a lower level of lactose. Thus, the two-strain hybrid process offers the advantage of obtaining higher levels of LST-c and reduced lactose levels. In addition, the hybrid process offers the advantage of replacing the expensive starting materials 6′SL and LNnT with cheap lactose and offering a higher conversion of LNnT to the desired product LST-c compared to the in vitro process, thus in all constituting a much more economical process.

### Example 8 Synthesis of LST-c Using a One Strain Hybrid Process

In addition to the two-strain hybrid process described in example 7, this example describes the synthesis of LST-c using the one strain hybrid process. In this one-strain hybrid process, the acceptor substrate LNnT was produced in situ by an LNnT strain (LNnT-S2 from table 12) from lactose and a purified sialyl donor substrate 6′-SL was added during the cultivation. The trans-sialylation reaction was catalyzed by adding α-2,6-transsialidase from Photobacterium leiognathi JT-SHIZ-119 (PITS-197, SEQ ID NO: 41).

The culture started with 700 g of mineral culture medium as described in the methods section above, containing lactose and 25 g/kg sucrose. The sucrose contained in the batch medium was depleted after approximately 15 h, after which a feed solution containing sucrose and minerals was fed continuously using a profile that kept the culture carbon limited, initially starting at a sucrose feed rate of 1.43 g/h and ramping up over 5 hours to 2.93 g/h whereafter it was kept constant. The temperature was initially set to 33° C. and was dropped to 30° C. with a linear 1 h ramp after 3 hours of feed. Sterile 6′SL was fed separately at a constant rate starting 15 hours into the fed-batch phase (approximately 30 hours after the inoculation) and lasting for 24 hours.

The transsialylation was started by adding 115 mg/L fermentation broth of α-2,6-transsialidase from Photobacterium leiognathi JT-SHIZ-119 (PITS-197, SEQ ID NO: 41) at 69.5 h after inoculation (start of the fermentation), when almost all the lactose had been converted into LNnT and the 6′SL had been added. Additional pulses of enzyme solution were added at 99 hours (366 mg/L), 121 hours (281 mg/L) and 146 hours (542 mg/L).

FIG. 10 shows the process progress curve in mass fraction of substrates and products relative to the total mass of substrates and products. As depicted, until the addition of the enzyme, lactose was nearly fully converted to LNnT by the E. coli strain. The increasing level of 6′-SL was due to the continued addition of the 6′-SL solution that lasted until the addition of the enzyme. From the point of enzyme addition at 69.5 h, the result of the enzymatic trans-sialylation can be seen in the formation of LST-c and depletion of 6′-SL. Moreover, the lactose concentration remained at a steady low level as the lactose side product released from the trans-sialylation reaction was quickly recycled by in vivo formation into LNnT. The performance of the one-strain hybrid production of LST-c was compared to the in vitro synthesis of LST-c from 6′-SL and LNnT as described in example 7 and shown at FIG. 8. Table 19 shows the comparison of the final product composition of LST-c synthesis using the one-strain hybrid vs. in vitro processes.

TABLE 19
Comparison of final product composition of the one-strain hybrid
process vs. in vitro process for the synthesis of LST-c

| | Lactose | 6′-SL | LNnT | LST-c |
|---|---|---|---|---|
| Process | (wt. %) | (wt. %) | (wt. %) | (wt. %) |
| In vitro | 11.9 | 17.3 | 31.8 | 38.8 |
| One-strain hybrid | 2.7 | 25.2 | 17.3 | 54.8 |

As can be seen in Table 19, the one-strain hybrid LST-c process led to a lower fraction of lactose and higher fraction of LST-c. Thus, like the two-strain hybrid process, the one-strain hybrid process offers the advantages of efficient process economy by obtaining higher fraction of the third oligosaccharide product LST-c and easier purification by the reduced lactose level.

### Example 9 Synthesis of LNFP-III Using a One-Strain Hybrid Process

The synthesis of LNFP-III in the one-strain hybrid process was performed with the 3FL producing strain 3FL-S2 from table 12 and external addition of LNnT and an α1,3/4-transfucosidase.

In the present example the 3FL strain was cultivated starting with 700 g of medium, lactose and 25 g/kg glucose as described in the Methods section above. After depletion of the glucose contained in the batch medium after approximately 15 h, a feed solution containing glucose and minerals was fed continuously using a profile that kept the culture carbon limited, initially starting at a feed rate of 0.88 g/h of glucose and ramping up over 5 hours to 1.76 g/h. This rate was kept for 92 h, after which it was increased to 3.61 g/h and kept there until the end of the cultivation. The temperature was initially set to 33° C. and was dropped to 30° C. with a linear 1 h ramp after 3 hours of feed. In this example, a 3:1 (mol/mol) excess of LNnT relative to lactose was added into the fermenter. The LNnT addition was started 15 h (approximately 30 hours after the inoculation) after the start of the fed-batch phase and lasted for 24 h. The trans-fucosylation of LNnT was started by adding 67 mg/L fermentation broth of sterile filtered mutated recombinant α1,3/4-transfucosidase from Bifidobacterium longum subsp. Infantis (BiTF-641, SEQ ID NO: 59) at 42 h after inoculation (start of the fermentation). Additional pulses of the enzyme solution were added at 97 h (48 mg/L) and 121 h (42 mg/L).

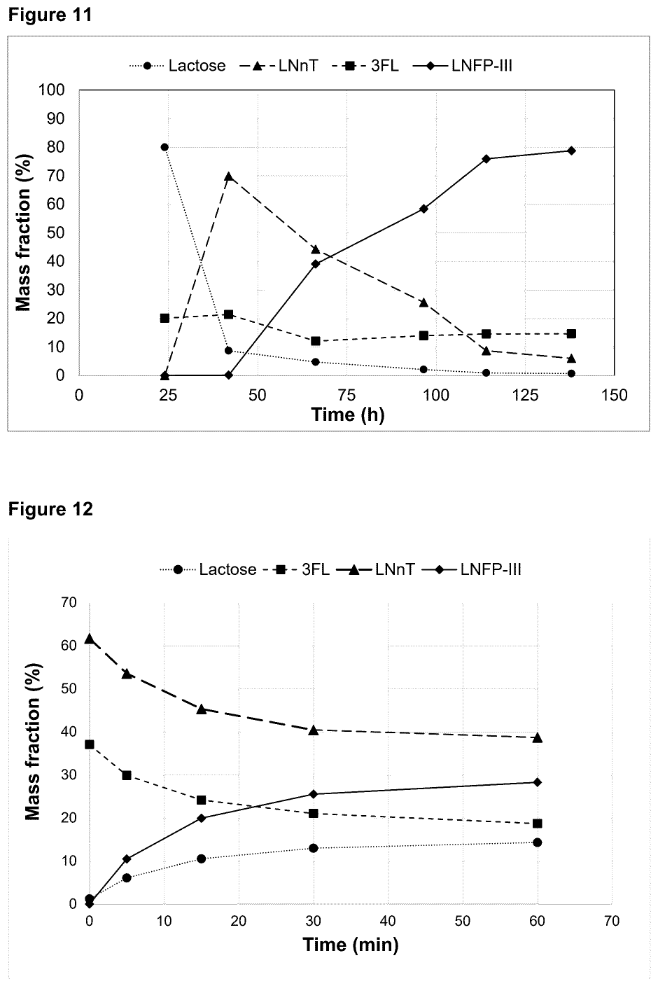

FIG. 11 illustrates the development of lactose, LNnT, 3FL and LNFP-III in weight % relative to the total weight of the substrates and products. 3FL3FL The trans-fucosylation of LNnT (acceptor) utilizing 3FL as a fucosyl donor was initiated by the addition of the BiTF-641 at 42 h which corresponds with the initiation of LNFP-III formation. From this point onwards LNFP-III accumulated while the LNnT levels decreased. Moreover, since the side-product lactose was subsequently taken up by the cell and recycled to 3FL, the process maintained a steady level of 3FL while reducing the initially added lactose. The one-strain hybrid LNPF-III synthesis led to 93.1% conversion of the LNnT. Table 20 shows the composition of lactose and HMOs at the end of the one-strain hybrid process.3FL

As in the previous examples, the one-strain hybrid process for the formation of LNFP-III was also compared to the conventional in vitro enzymatic process for forming LNFP-III as described here.

A purely in vitro enzymatic synthesis of LNFP-III was carried out using BiTF-641 (SEQ ID NO: 59) catalyzed trans-fucosylation of LNnT utilizing 3FL as a fucosyl donor starting from 3:1 mol/mol LNnT:3FL ratio. A substrate solution consisting of 200 mM LNnT and 66.7 mM 3FL was prepared at pH 6.5. The trans-fucosylation reaction was started by adding 0.4 mg/ml BiTF-641 at 30° C. The progress of the reaction was monitored by measuring the concentrations of substrates and products with HPLC. Samples for the HPLC analysis were collected and immediately heat-treated at 95° C. for 5 min to denature the BiTF-641 enzyme to stop the reaction. FIG. 12 shows the progress of the in vitro reaction for the synthesis of LNFP-III starting from 3:1 mol/mol LNnT:3FL ratio as mass fractions of substrates and products relative to the total mass of substrates and products. The in vitro reaction progress stopped at equilibrium after 34.6%. From FIG. 12, it can be seen that for the in vitro process performed at a high acceptor to donor substrate ratio, the extent of product formation is limited to the limiting substrate conversion, resulting in a very high fraction of the unconverted acceptor substrate LNnT and low fraction of the third product oligosaccharide LNFP-III in the final product mixture as shown in table 20. In the hybrid process on the other hand, the side-product lactose is recycled back to the donor substrate in the circular loop of the combined fermentation and enzymatic steps, which allows a high conversion of the excess acceptor substrate (LNnT) leading to a high fraction of the third oligosaccharide product (LNFP-III). Table 20 shows a comparison of the final product composition of LNFP-III synthesis using the one-strain hybrid vs. in vitro processes from starting at 3-fold higher molar amount of LNnT relative to lactose in the hybrid process and relative to 3FL in the in vitro process.

TABLE 20
Comparison of final product composition of the one-strain hybrid
process vs. in vitro process for the synthesis of LNFP-III starting
from a 3-fold excess molar amount of LNnT relative to lactose in
the hybrid process and relative to 3FL in the in vitro process

| | | | | | LNnT |
|---|---|---|---|---|---|
| | Lactose | 3FL | LNnT | LNFP-III | conversion |
| Process | (wt. %) | (wt. %) | (wt. %) | (wt. %) | (%) |
| In vitro | 11.7 | 6.9 | 60.1 | 21.3 | 34.6 |
| One-strain | 0.7 | 14.6 | 6.0 | 78.7 | 93.1 |
| hybrid | | | | | |

Table 20 shows that the one-strain hybrid process produces more than 3.5 fold more LNFP-III than the conventional in vitro processes when starting at 3-fold higher molar amount of LNnT relative to lactose in the hybrid process and relative to 3FL in the in vitro process. Also the by-product, lactose is 15 fold lower in the one-strain hybrid process than in the in vitro process.

Specifically, the one-strain hybrid LNPF-III synthesis led to 93.1% conversion of the LNnT, which was added in a 3-fold excess relative to lactose (mol/mol). In contrast, as described in the comparative example, an in vitro enzymatic LNFP-III synthesis starting from 3:1 mol/mol LNnT:3FL ratio only led to a 34.6% conversion of the supplied LNnT.

This large difference in conversion rate between the two processes is due to the side-product from the transfucosylation reaction, lactose, is recycled back to the cell producing the donor substrate (3FL) in the circular loop of the combined fermentation and enzymatic steps, which allows a high conversion of the excess acceptor substrate (LNnT) leading to a high fraction of the third oligosaccharide product (LNFP-Ill). Thus, the one-strain hybrid process, offers the advantages of efficient process economy by obtaining higher fraction of the third oligosaccharide product LNFP-III and easier purification by the reduced lactose and LNnT levels.

### Example 10 Synthesis of LNFP-II Using a One Strain Hybrid Process

The synthesis of LNFP-II in the one-strain hybrid process was performed with the 3FL producing strain 3FL-S1 from table 12 and external addition of LNT and an α1,3/4-transfucosidase.

In the present example the 3FL strain was fermented starting with 700 g of medium, lactose and 25 g/kg glucose as described in the Methods section above. After depletion of the glucose contained in the batch medium after approximately 13 h, a feed solution containing glucose and minerals was fed continuously using a profile that kept the culture carbon limited, initially starting at a glucose feed rate of 1.5 g/h and ramping up over 5 hours to 3 g/h and kept there until the end of the cultivation. The temperature was initially set to 33° C. and was dropped to 30° C. with a linear 1 h ramp after 3 hours of feed. In this example, a two-fold excess molar amount of LNT relative to lactose was added into the fermenter over the course of the fermentation. Whereas the lactose was fully supplied form the beginning, the LNT addition was started 15 h after the start of the fed-batch phase and lasted for 24 h. The trans-fucosylation of LNT was started by adding 66 mg/L fermentation broth of sterile filtered mutated recombinant α1,3/4-transfucosidase from Bifidobacterium longum subsp. Infantis (BiTF-641, SEQ ID NO: 59) at 43 h after the start of the fermentation.

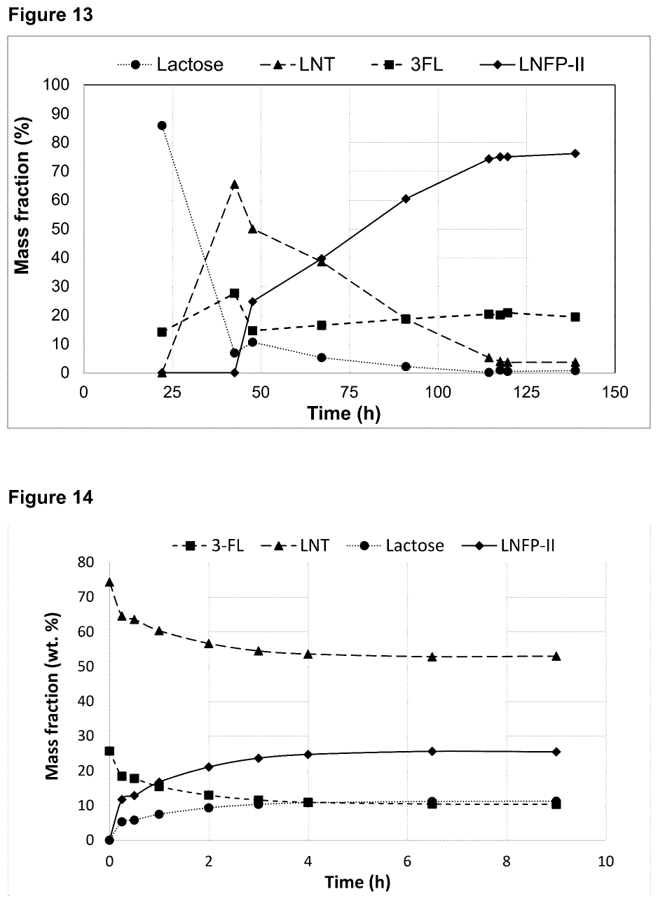

FIG. 13 illustrates the development of lactose, LNT, 3FL and LNFP-II in weight % relative to the total weight of the substrates and products. 3FLFucosylation of LNT by BiTF-641 utilizing 3FL as a fucosyl donor started right after the enzyme addition at 42 h, and could be observed in the accumulating levels of LNFP-II and decreasing levels of LNT. Immediately after the initiation of the trans-fucosylation reaction the concentration of 3FL decreased and lactose increased as it was released from the fucosyl-donor, 3FL, due to the initially very rapid enzymatic rate when the product concentrations were still low. The one-strain hybrid LNPF-II synthesis led to 95.5% conversion of LNT from a starting point of 2:1 molar amount of LNT relative to lactose. Table 21 shows the composition of lactose and HMOs at the end of the one-strain hybrid process.

As in the previous examples, the one-strain hybrid process for the formation of LNFP-II was also compared to the conventional in vitro enzymatic process for forming LNFP-II as described here.

3FLA purely in vitro enzymatic synthesis of LNFP-II was carried out using BiTF-641 (SEQ ID NO: 59) catalyzed trans-fucosylation of LNT utilizing 3FL as a fucosyl donor starting from 1:1 or 2:1 mol/mol LNT:3FL ratio. A substrate solution consisting of 200 mM LNT and 100 mM 3FL or 150 mM LNT and 150 mM 3FL was prepared at pH 6.7. The trans-fucosylation reaction was started by adding 0.51 mg/ml BiTF-641 at 25° C. The progress of the reaction was monitored by measuring the concentrations of substrates and products with HPLC. Samples for the HPLC analysis were collected and immediately heat-treated at 95° C. for 5 min to denature the BiTF-641 enzyme and stop the reaction. FIG. 14 shows the progress of the in vitro synthesis of LNFP-II starting from 2:1 mol/mol LNT:3FL ratio in mass fractions of substrates and products relative to the total mass of substrates and products. The in vitro process with the same acceptor:donor ratio (LNT:3FL in 2:1) as the hybrid process only reached 28.5% conversion of the LNT that had been supplied. Table 21 shows a comparison of the final product composition of LNFP-II synthesis using the one-strain hybrid vs. in vitro processes when starting at 2-fold higher molar amount of LNT relative to lactose in the hybrid process and 1:1 or 2:1 mol/mol of LNT/3FL in the in vitro process.

TABLE 21
Comparison of final product composition of the one-strain hybrid
process vs. in vitro process for the synthesis of LNFP-II

| | | | | | LNT |
|---|---|---|---|---|---|
| | Lactose | 3FL | LNT | LNFP-II | conversion |
| Process | (wt. %) | (wt. %) | (wt. %) | (wt. %) | (%) |
| In vitro (1:1 | 20.6 | 26.4 | 30.8 | 22.2 | 57.4 |
| LNT:3FL) | | | | | |
| In vitro (2:1 | 11.2 | 10.3 | 53.0 | 25.4 | 28.5 |
| LNT:3FL) | | | | | |
| One-strain hybrid | 0.7 | 19.4 | 3.7 | 76.2 | 95.5 |

Table 21 shows that the one-strain hybrid process produces at least 3 fold more LNFP-II than the conventional in vitro process both when the acceptor:donor ratio is 1:1 and 2:1. Also, the by-product, lactose is at least 16 fold lower in the one-strain hybrid process compared to the in vitro process.

Specifically, the one-strain hybrid LNPF-II synthesis led to 95.5% conversion of the LNT, which was added in a 2-fold excess relative to lactose (mol/mol). In contrast, as described in the comparative example, an in vitro enzymatic LNFP-II synthesis starting from 2:1 mol/mol LNT:3FL ratio only led to a 28.5% conversion of the supplied LNT. When the in vitro was run at 1:1 ratio the conversion of LNT improved significantly, clearly illustrating the in vitro process is limited by the kinetics, in that it reaches an equilibrium, and this cannot be significantly changed by increasing the amount of acceptor in the process.

Thus, the one-strain hybrid process, offers the advantages of efficient process economy by obtaining higher fraction of the third oligosaccharide product, LNFP-II, and easier purification by the reduced lactose and LNT levels.

### Example 11 Synthesis of LST-a Using a One-Strain Hybrid Process

This example describes the synthesis of LST-a using the one-strain hybrid process. The process was performed with the LNT producing strain LNT-S1 from table 12 and by adding a purified sialyl donor substrate 3′SL during the cultivation and an α-2,3-transsialidase.

The synthesis of LST-a in the one-strain hybrid process was performed with the LNT-S1 strain from table 12. In the present example the LNT strain was cultivated starting with 700 g of medium, lactose and 25 g/kg sucrose as described in the Methods section above. After depletion of the sucrose contained in the batch medium after approximately 12 h, a feed solution containing sucrose and minerals was fed continuously using a profile that kept the culture carbon limited, initially starting at a sucrose feed rate of 1.46 g/h and ramping up over 5 hours to 2.9 g/h and kept there until the end of the cultivation. The temperature was initially set to 33° C. and was dropped to 30° C. with a linear 1 h ramp after 3 hours of feed. In this example, a 2:1 total molar amount of 3′SL relative to lactose was added into the fermenter. The 3′SL addition was started 15 h after the start of the fed-batch phase (approximately 27 hours after the inoculation/fermentation start) and lasted for 24 h. The trans-sialylation of LNT was started 264 mg/L fermentation broth of sterile filtered α-2,3-transsialidase (TcTS, SEQ ID NO: 60) 49 h after the start of the fermentation.

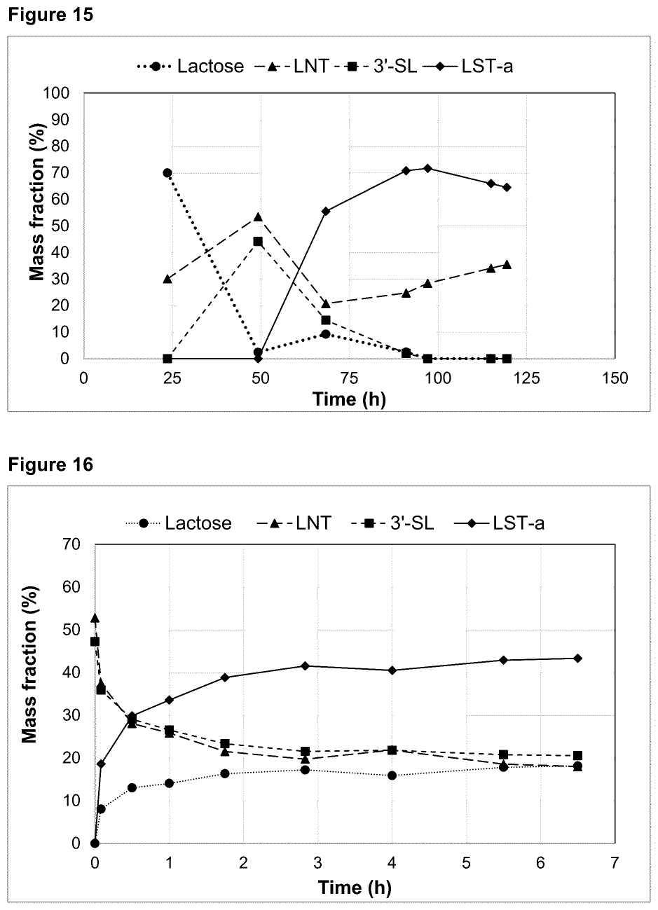

FIG. 15 shows the development of lactose, LNT, 3′SL and LST-a in weight % relative to the total weight of the substrates and products. Sialylation of LNT by TcTS utilizing 3′SL as a sialyl donor started right after the enzyme addition at 49 h, and could be observed in the accumulating levels of LST-a and decreasing levels of LNT and 3′SL. After the addition of the enzyme, the formation of LST-a commenced as could also be seen by the declining level, and in the end, complete depletion of 3′SL. Immediately after the initiation of the trans-sialyation reaction the concentration of lactose increased as it was released from the sialyl-donor, 3′SL, due to the initially very rapid enzymatic rate when the product concentrations were still low. The one-strain hybrid LST-a synthesis led to 100% conversion of 3′SL from a starting point of 2:1 molar amount of 3′SL relative to lactose. Table 22 shows the composition of lactose and HMOs at the end of the one-strain hybrid process.

As in the previous examples, the one-strain hybrid process for the formation of LST-a was also compared to the conventional in vitro enzymatic process for forming LST-a as described here.

The comparative in vitro experiment was performed using purified LNT and 3′SL as substrates. A substrate solution consisting of 150 mM LNT and 150 mM 3′SL was prepared at pH 6.5 and 25° C. The trans-sialylation reaction was started by adding 0.51 mg/mL α-2,3-transsialidase (TcTS, SEQ ID NO: 60). The progress of the reaction was monitored by measuring the concentrations of substrates and products by HPLC. Samples for the HPLC analysis were collected and immediately heat-treated at 90° C. for 5 min in order to quench the reaction. FIG. 16 shows the progress of the in vitro trans-sialylation of LNT using 3′SL as a sialyl donor for the synthesis of LST-a. In contrast to the hybrid process that led to a full conversion of 3′SL even when starting with two-fold higher (mol/mol) 3′SL level relative to lactose, the in vitro process only achieved 57% 3′SL conversion starting at 1:1 mol/mol of 3′SL/LNT. Table 22 shows a comparison of the final product composition of LST-a synthesis using the one-strain hybrid vs. in vitro processes starting at 2:1 molar ratio of 3′SL relative to lactose in the hybrid process and 1:1 molar ratio of 3′SL relative to LNT in the in vitro process.

TABLE 22
Comparison of final product composition of the one strain
hybrid process vs. in vitro process for the synthesis of
LST-a when starting at 2-fold higher molar amount of 3′SL
relative to lactose in the hybrid process and 1:1 molar
ratio of 3′SL relative to LNT in the in vitro process

| | | | | | 3′SL |
|---|---|---|---|---|---|
| | Lactose | 3′SL | LNT | LST-a | conversion |
| Process | (wt. %) | (wt. %) | (wt. %) | (wt. %) | (%) |
| In vitro | 18.1 | 20.5 | 17.9 | 43.3 | 57 |
| One-strain hybrid | 0 | 0 | 35.5 | 64.5 | 100 |

Table 22 shows that the one-strain hybrid process produces 1.5 fold more LST-a than the conventional in vitro process. Also, the by-product, lactose, is reduced to 0 in the one-strain hybrid process whereas this remains at 18% in the in vitro process.

Specifically, the one-strain hybrid LST-a synthesis led to 100% conversion of the 3′SL, which was added in a 2-fold excess relative to lactose (mol/mol). In contrast, as described in the comparative example, an in vitro enzymatic LST-a synthesis starting from 1:1 mol/mol 3′SL:LNT ratio only led to a 57% conversion of the supplied LNT.

As the hybrid process can produce a full conversion of the acidic (i.e. ionizable) substrate 3′SL, it offers a large advantage in purification as it is easy to separate the acidic (i.e. ionizable) product LST-a from the neutral LNT.

### Example 12 Synthesis of LST-a Using a Two-Strain Hybrid Process

In addition to the one-strain hybrid process for the synthesis of LST-a as described in example 11, this example describes the synthesis of LST-a using the two-strain hybrid process. The two strains, one producing LNT from lactose (substrate) and sucrose (carbon source) and the second producing 3-′SL from lactose (substrate) and glucose (carbons source) were co-cultured. The 3′SL was produced as a sialyl donor substrate and the LNT as an acceptor substrate for a subsequent trans-sialyation reaction to LST-a catalyzed by a α-2,3-transsialidase (TcTS, SEQ ID NO: 60) added to the medium.

The synthesis of LST-a in the two-strain hybrid process was performed with the LNT producing strain LNT-S1 and the 3′SL producing strain 3′SL-S1 from table 12. In the present example the strains were co-cultivated starting with 700 g of medium, lactose, 15 g/kg glucose and 15 g/kg sucrose as described in the Methods section above. The co-culture was initiated with 1% (v/v) inoculum from each strain grown in pre-cultures with similar medium containing sucrose for the LNT strain and glucose for the 3′SL strain, both grown to an OD₆₀₀ of 2.5-5. After depletion of the glucose and sucrose contained in the batch medium after approximately 18 h, separate glucose and a sucrose mineral feed solutions were fed continuously at a rate of 1.17 g glucose/h and 1.17 g sucrose/h, which kept the co-culture carbon limited. The temperature was initially set to 33° C. and was dropped to 28° C. with a linear 1 h ramp at the start of the fed-batch phase. The trans-sialylation of LNT was started by the addition of 339 mg/L fermentation broth of sterile filtered α-2,3-transsialidase (TcTS, SEQ ID NO: 60) 68 h after the start of the fermentation and adding another pulse of the enzyme solution at 90 h.

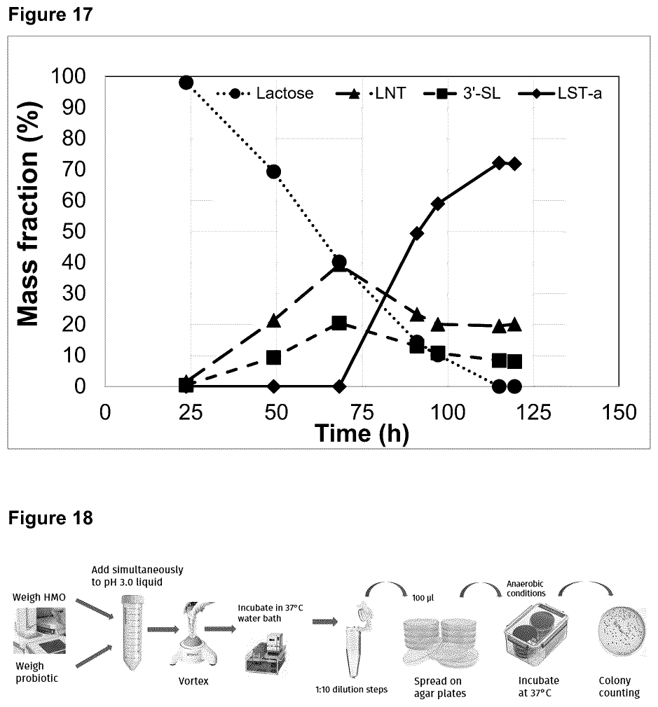

FIG. 17 shows the development of lactose, LNT, 3′SL and LST-a in weight % relative to the total weight of the substrates and products. The data until 68 h of the process (i.e., the addition of the enzyme) show the conversion of lactose to LNT and 3′SL by LNT strain and 3′SL strain, respectively, with no formation of LST-a. After the addition of the enzyme at 68 h, LNT and 3′SL decreased as increasing amounts of LST-a was formed. Moreover, even though lactose was released as a side product from the enzymatic reaction its concentration kept decreasing as it was rapidly recycled into LNT and 3′SL by the corresponding strains. The two-strain hybrid process for LST-a synthesis achieved full utilization of lactose and a higher LST-a fraction in contrast to the purely in vitro LST-a process as shown in table 23. Table 23 shows a comparison of the final product composition of LST-a synthesis using the two-strain hybrid vs. in vitro processes.

TABLE 23
Comparison of final product composition of the two-strain hybrid
process vs. in vitro process for the synthesis of LST-a

| | Lactose | 3′SL | LNT | LST-a |
|---|---|---|---|---|
| Process | (wt. %) | (wt. %) | (wt. %) | (wt. %) |
| In vitro | 18.1 | 20.5 | 17.9 | 43.3 |
| Two-strain hybrid | 0 | 8.0 | 20.1 | 71.8 |

From table 23 it can be seen that the two-strain hybrid process for LST-a synthesis achieved full utilization of lactose and a higher LST-a fraction in contrast to the purely in vitro LST-a process. Also the amount of 3′SL in the final mixture produced by the two-strain hybrid process is 2.5 times lower which offers an advantage in purification it is more challenging to separate 3′SL from LST-a than the neutral LNT.

### Example 13—Regeneration and Viability of Lyophilized Lactobacillus Species

Probiotics may be consumed as live bacteria or as a dried (e.g. lyophilized) product. Independent of the drying method, rehydration involves an important step in the recovery of dehydrated bacteria; an inadequate rehydration/regeneration step may lead to poor cell viability and a low final survival rate. Rehydration is therefore a highly critical step in the revitalization of a lyophilized culture. For both live and rehydrated bacteria, the survival of the bacteria under acidic conditions is critical since they need to pass through the acidic environment of the stomach and may also be faced with storage (shelf-life) in acidic food products.

In the present example it was tested whether the mixture of HMOs similar to the ones produced by the hybrid processes described in examples 1, 7, 8, 11 and 12 can provide a benefit in the rehydration (regeneration) and viability of the probiotics. The test was performed under acidic conditions to resemble the conditions bacteria have to survive when passing through the stomach or when dosed in an acidic beverage.

The lyophilized probiotic, Lactobacillus rhamnosus DSM 32550 (0.4 mg/ml), alone (control) or in combination with HMO mixtures (5% w/v) as indicated in table 24, were dissolved in sterile phosphate-buffered saline (PBS, pH=3), warmed to 37° C. and vigorously mixed for about 30 sec until no visible clumps remained. The tubes were incubated at 37° C. for 3 h. The samples were further diluted and 100 μl were spread in duplicates onto MRS agar plates which were incubated at 37° C. in anaerobic chambers. For the experimental setup, see FIG. 18.

TABLE 24
HMO compositions tested in the present example

| HMO | C | Mix5 | Mix6 | Mix7 | Mix8 |
|---|---|---|---|---|---|
| LST-a | 0 | 55 | 65 | 0 | 0 |
| LNT | 0 | 45 | 25 | 0 | 0 |
| 3′SL | 0 | 0 | 10 | 0 | 0 |
| 3FL | 0 | 0 | 0 | 45 | 0 |
| FSL | 0 | 0 | 0 | 55 | 0 |
| LNnT | 0 | 0 | 0 | 0 | 25 |
| LST-c | 0 | 0 | 0 | 0 | 50 |
| 6′SL | 0 | 0 | 0 | 0 | 25 |

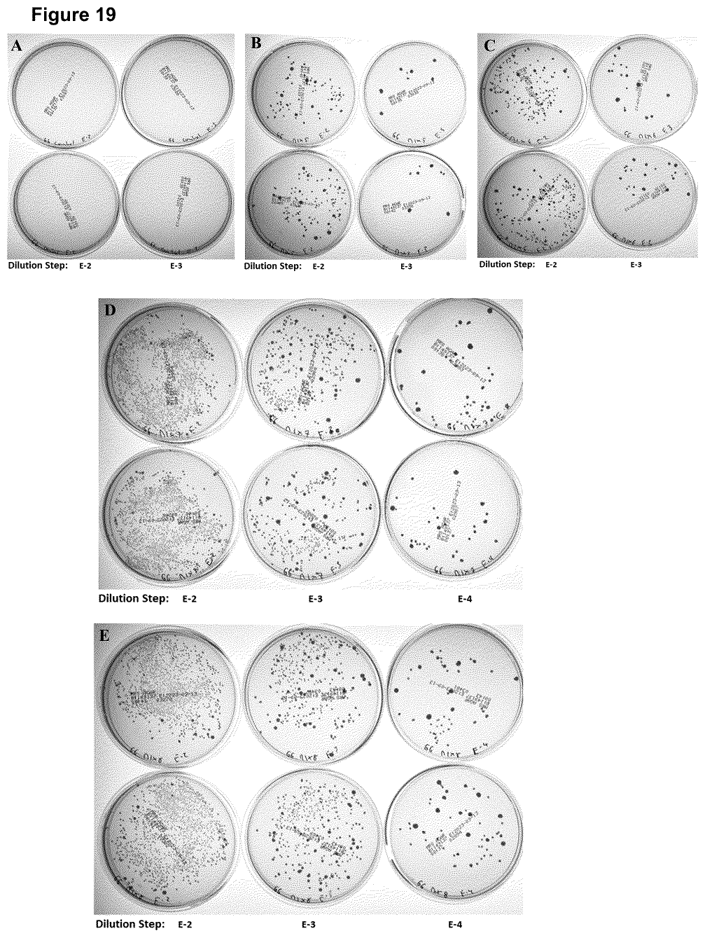

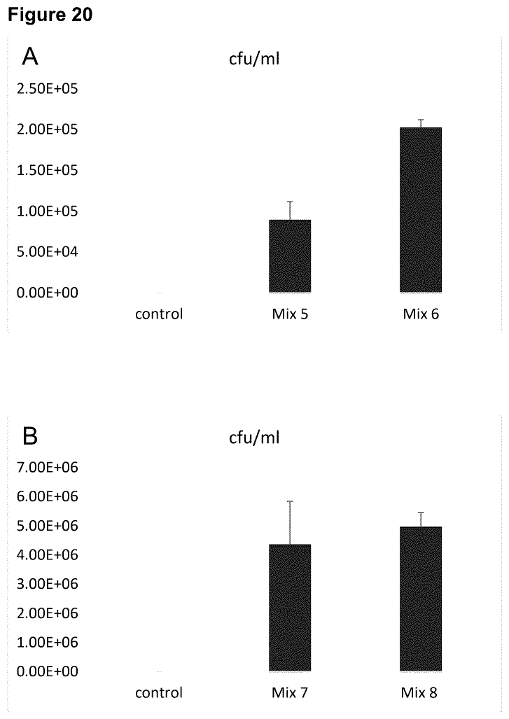

FIG. 19 shows picture of the plates with the colonies of Lactobacillus rhamnosus DSM 32550 after 6 days incubation. The CFU/ml was calculated based on colonies counted 72 hours after incubation (average of two plates). The E-2 dilution plates were used for counting mix 5 and mix 6, results shown in FIG. 20A (LST-a containing mixtures) and E-4 dilution plates were used for counting mix 7 and mix 8, results shown in FIG. 20B.

Lyophilized Lactobacillus rhamnosus DSM 32550 dissolved with the HMO mixtures described herein showed an enhanced regeneration and survivability compared to control without the HMO mixtures, where survival was 0. These data clearly show that the regeneration and viability of Lactobacillus rhamnosus DSM 32550 after exposure to low pH conditions, such as in the stomach or in an acidic beverage, can be improved in the presence of any of the HMO mixtures. It can also be seen that a higher LST-a amount in combination with some 3′SL performs better than just the mixture of LST-a and LNT. Mixture 7 (3FL and FSL) and mixture 8 (LST-c, LNnT and 6′SL) are both more potent in terms of regeneration and viability of Lactobacillus rhamnosus DSM 32550 compared to mixture 5 and 6.

To our knowledge it has not previously been shown that the tested mixtures provides a benefit of improving the regeneration and survivability of probiotics in an acidic environment.

### Example 14—Regeneration and Viability of Lyophilized Bifidobacterium Species

As in example 13 above the HMO mixtures in table 24 were also tested for their ability to provides a benefit of improving the regeneration and survivability of Bifidobacterium longum DSM 32946 in an acidic environment.

Lyophilized probiotic, Bifidobacterium longum DSM 32946 (0.4 mg/ml), alone or in combination with HMOs mixtures (5% w/v) as indicated in table 24, were dissolved into sterile pH 3.0 water, warmed to 37° C., and vigorously mixed for about 30 seconds until no visible clumps remained. The tubes were incubated at 37° C. for 30 minutes. Afterwards 100 μl were spread in duplicates onto MRS cysteine agar plates which were incubated for 48 h at 37° C. in anaerobic chambers. The regeneration and viability of the probiotics were determined by counting the colonies on the plates after 48 h of incubation.

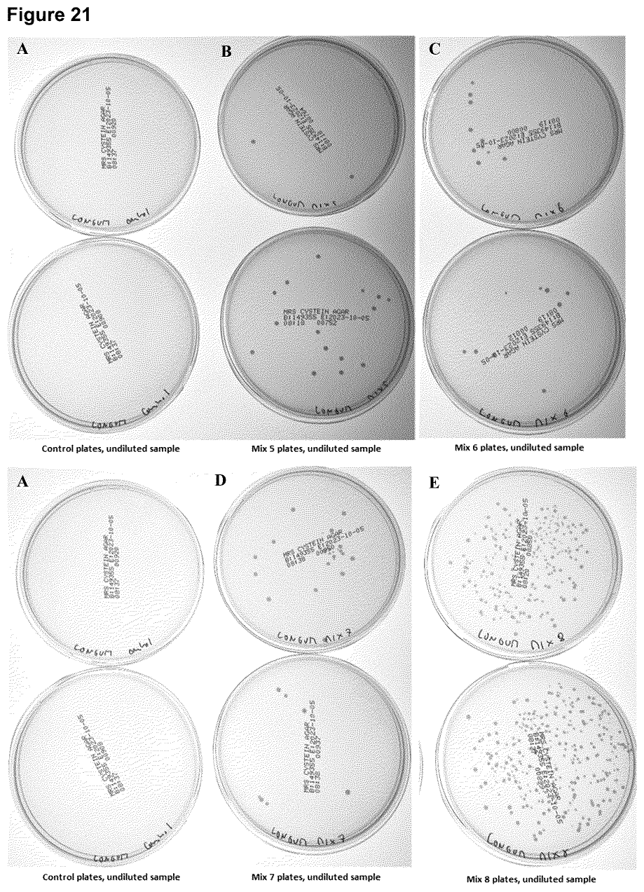

FIG. 21 shows picture of the plates with the colonies of Bifidobacterium longum DSM 32946 after 2 days incubation. The CFU/ml was calculated based on colonies counted on undiluted plates 48 hours after incubation (average of two plates). The results are shown in table 25.

TABLE 25
Average CFU/ml of Bifidobacterium longum DSM 32946 after 30
min acid treatment and 48 h subsequent incubation at 37° C.

| Mixture | control | mix5 | mix6 | mix7 | mix8 |
|---|---|---|---|---|---|
| CFU/ml Average | 0 | 85 | 95 | 165 | 3245 |

As can be seen from table 25, the mixtures are capable making some Bifidobacterium longum DSM 32946 strains survive acid treatment compared to the control where the survival rate is 0.

1. A method for producing an oligosaccharide from a donor oligosaccharide and an acceptor oligosaccharide or acceptor disaccharide, comprising the steps of:
a) cultivating a genetically modified cell capable of producing a first oligosaccharide of at least three monosaccharide units in a culture medium which is supplied with a carbon source, and wherein the genetically modified cell comprises one or more nucleic acids encoding:
i) at least one side-product importer, and
ii) at least one recombinant glycosyltransferase, and
iii) at least one pathway to produce a nucleotide-activated sugar, and
b) supplying a second disaccharide or oligosaccharide to the culture medium, and
c) making an enzyme with transglycosidase activity available in the culture medium, and
d) incubating the first oligosaccharide, the second disaccharide or oligosaccharide and the transglycosidase enzyme in the culture medium in which the first oligosaccharide is produced to form a third oligosaccharide.

2. The method according to claim 1, wherein the second oligosaccharide or disaccharide supplied to the culture medium is produced in a separate process or produced by the genetically modified cell in step a).

3. The method according to claim 1, wherein the second oligosaccharide or disaccharide is supplied to the culture medium from a second genetically modified cell present in the same culture as the genetically modified cell in step a) and the second genetically modified cell is capable of producing the second oligosaccharide or disaccharide.

4. The method according to claim 1, wherein the genetically modified cell(s) exports the first and/or second oligosaccharide or disaccharide produced by the cell(s) into the culture medium.

5. The method according to claim 1, wherein the transglycosidase enzyme is either added to the culture medium during the cultivation or is expressed from a recombinant nucleic acid in said genetically modified cell.

6. The method according to claim 1, wherein the transglycosidase enzyme is selected from the group consisting of α-1,2-tranfucosidase, α-1,3-transfucosidase, α-1,3/4-transfucosidase, α-2,3-transialylase, α-2,6-transsialylase, β-N-acetylglucosaminidase, trans-lacto-N-biosidase and trans-β-galactosidase.

7. The method according to claim 1, wherein the donor oligosaccharide is selected from the group consisting of a fucosylated oligosaccharide of three to five monosaccharide units, a sialylated oligosaccharide of three to five monosaccharide units and a neutral core oligosaccharide three to four monosaccharide units.

8. The method according to claim 1, wherein the cultivation in step a) is initiated in the presence of sufficient initial substrate for producing the first oligosaccharide.

9. The method according to claim 5, wherein the transglycosidase is added to the culture at a time point when the genetically modified cell has converted at least 50% of the initial substrate for producing the first oligosaccharide into the first oligosaccharide.

10. The method according to claim 9, wherein the initial substrate for producing the first oligosaccharide is lactose, N-acetyllactosamine, lacto-N-biose or 2′FL.

11. The method according to claim 8, wherein the second oligosaccharide or disaccharide is added to the culture medium and the molar amount of the second oligosaccharide is at least 2:1 of the initial substrate added for producing the first oligosaccharide.

12. The method according to claim 1, wherein the at least one side-product importer comprises LacY comprising the amino acid sequence of SEQ ID NO: 3, or a functional variant thereof.

13. The method according to claim 1, wherein the third oligosaccharide is a complex oligosaccharide of at least four monosaccharide units selected from the group consisting of DFL, FSL, Lewis B, Lewis Y, sialyl-Lewis A, sialyl-Lewis X, LNFP-I, LNFP-II, LNFP-III, LNFP-IV, LNFP-V, LNFP-VI, LST-a, LST-b, LST-c, DSLNT, LNDFH-I, LNDFH-II, LNDFH-III, FLST-a, FLST-b, FLST-c, pLNH, pLNnH, LNH, LNnH, FLNH-I, FLNH-II, FLNH-III, FpLNH-I, FpLNnH II, DF-LNF-I, DF-LNF-II, DF-LNF-III, DF-para-LNH, DF-para LNnH, FLNnHa, FLNnHb, DFLNnH, TF-LNH, SLNH, FSLNH, SLNnH-I, FSLNnH-I, SLNnH-II, and DS-FLNH-II.

14. The method according to claim 1, wherein the genetically modified cell produces a donor oligosaccharide selected from the group consisting of 2′FL, 3FL, DFL, LNFP-I, 3′SL, 6′SL, 3′SLacNAc, 3′SLNB, FSL, LST-a, LNT-II, LNT and LNnT.

15. The method according to claim 1, wherein the genetically modified cell produces an acceptor oligosaccharide selected form the group consisting of 2′FL, 3FL, 2′FLacNAc, 2′FLNB, Lewis A, Lewis X, LNT-II, LNT, LNnT, Para-LNnH, LNFP-I, LNFP-II, LNFP-III, LNFP-IV, LNFP-V, LNFP-VI, 3′SL, 6′SL, LST-a, and LST-c.

16. The method according to claim 1, wherein the first, second and third oligosaccharides are human milk oligosaccharides (HMOs).

17. The method according to claim 1, wherein the at least one recombinant glycosyltransferase is selected from the group consisting of α-1,2-fucosyltransferase, α-1,3-fucosyltransferase, α-1,3/4-fucosyltransferase, α-2,3-sialyltransferase, α-2,6-sialyltransferase β-1,3-N-acetylglucosaminyltransferase, β-1,6-N-acetylglucosaminyltransferase, β-1,3-galactosyltransferase and β-1,4-galactosyltransferase.

18. The method according to any claim 1, wherein the at least one pathway to produce a nucleotide-activated sugar comprises the de novo GDP-fucose pathway (gmd, wcaG, manB, manC and manA) or the sialic acid sugar nucleotide pathway (neuB, neuC and neuA).

19. The method according to claim 1 claims, wherein the second disaccharide or oligosaccharide is selected from the group consisting of LacNAc, LNB, 2′FL, 3FL, 2′FLacNAc, 2′FLNB, Lewis A, Lewis X, 3′SL, LNT, LNnT, LNFP-I, LNFP-II, LNFP-III, LNFP-V, LNFP-VI, LSTa, LSTc, LNH, F-LNH-II, F-LNH-III, DF-LNH-I, DF-LNH-II, DF-LNH-III, S-LNH, DS-LNH, FS-LNH, LNnH, Para-LNH and Para-LNnH.

20. A composition of HMOs consisting essentially of:
e) at least 50 wt % FSL, below 45 wt % 3FL, and below 2 wt % 3′SL and below 3 wt % lactose, or
f) at least 55 wt % LST-a, below 40 wt % LNT, below 10 wt % 3′SL and below 2 wt % lactose, or
g) at least 40 wt % LST-c, below 25 wt % LNnT, below 25 wt % 6′SL and below 10 wt % lactose,
where in the total composition constitute 100 wt % of the components and the composition is a mixture of at least two components.

21.-22. (canceled)
